(19) United States
(12) Patent Application Publication
(10) Pub. No.: US 2026/0199501 A1
(43) Pub. Date: Jul. 16, 2026

(54) BISPECIFIC ANTI-EGFR/C-MET ANTIBODY-DRUG CONJUGATES

(71) Applicant: Janssen Biotech, Inc., Horsham, PA (US)

(72) Inventors: Benjamin Henley, Mount Laurel, NJ (US); Linxiao Chen, Media, PA (US); Shalom Goldberg, Merion Station, PA (US); Neeraj Kohli, Arlington, MA (US); Smruthi Vijayaraghavan, Lansdale, PA (US); Sylvie Laquerre, Chesterbrook, PA (US); Sheri Moores, Wayne, PA (US); Suresh Kumar Swaminathan, Lansdale, PA (US)

(21) Appl. No.: 19/446,412

(22) Filed: Jan. 12, 2026

Related U.S. Application Data
(60) Provisional application No. 63/744,531, filed on Jan. 13, 2025.
(60) Provisional application No. 63/857,106, filed on Aug. 4, 2025.
(60) Provisional application No. 63/872,442, filed on Aug. 29, 2025.

(51) Int. Cl. A61K 47/68; A61P 35/00

(52) U.S. Cl. CPC A61K 47/68037; A61K 47/6849; A61P 35/00

(57) ABSTRACT

This disclosure relates to bispecific anti-EGFR/c-Met antibody-drug conjugates and methods for treating cancer using the same.

### CROSS-REFERENCE TO RELATED APPLICATIONS

This application claims priority to and the benefit of U.S. Provisional Patent Application No. 63/744,531, filed Jan. 13, 2025, U.S. Provisional Patent Application No. 63/857,106, filed Aug. 4, 2025, and U.S. Provisional Patent Application No. 63/872,442, filed Aug. 29, 2025, the disclosures of which are incorporated herein by reference in their entirety.

### SEQUENCE LISTING

The instant application contains a Sequence Listing which is being submitted herewith electronically in XML format and is hereby incorporated by reference in its entirety. Said XML copy, created on Jan. 12, 2026, is named JBI6976USNP1_SL.xml and is 60,119 bytes in size.

### TECHNICAL FIELD

Disclosed herein are bispecific anti-EGFR/c-Met antibody-drug conjugates and methods for treating cancer using the same.

### BACKGROUND

Current small molecule and large molecule therapeutic approaches to antagonize EGFR or c-Met signaling pathways for therapy may be sub-optimal due to possible lack of specificity, potential off-target activity, and dose-limiting toxicity that may be encountered with small molecule inhibitors. Typical monospecific bivalent antibodies may result in clustering of membrane bound receptors and unwanted activation of the downstream signaling pathways. Monovalent antibodies having full length heavy chains (half arms) pose significant complexity and cost to the manufacturing process.

### SUMMARY

Disclosed herein are bispecific anti-EGFR/c-Met antibody-drug conjugates (ADCs), comprising a bispecific anti-EGFR/c-Met antibody that comprises a first heavy chain (HC1) comprising a heavy chain complementarity determining region 1 (HCDR1) comprising the amino acid sequence of SEQ ID NO: 1, a HCDR2 comprising the amino acid sequence of SEQ ID NO: 2, and a HCDR3 comprising the amino acid sequence of SEQ ID NO: 3; a first light chain (LC1) comprising a light chain complementarity determining region 1 (LCDR1) comprising the amino acid sequence of SEQ ID NO: 4, a LCDR2 comprising the amino acid sequence of SEQ ID NO: 5, and a LCDR3 comprising the amino acid sequence of SEQ ID NO: 6; a second heavy chain (HC2) comprising a HCDR1 comprising the amino acid sequence of SEQ ID NO: 7, a HCDR2 comprising the amino acid sequence of SEQ ID NO: 8, and a HCDR3 comprising the amino acid sequence of SEQ ID NO: 9; and a second light chain (LC2) comprising a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12; and linker-payload (compound I (CPT-113)) having the structure:

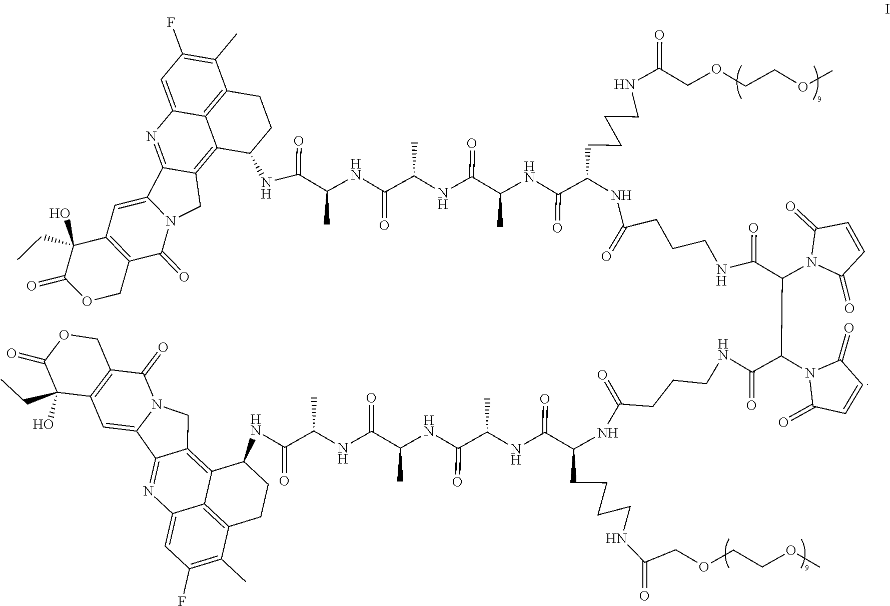

Disclosed herein are bispecific anti-EGFR/c-Met antibody-drug conjugates (ADCs), comprising a bispecific anti-EGFR/c-Met antibody that is conjugated or connected to the linker-payload through the cysteine residues of said antibody to either carbon of the CH₂ of the 5 membered succinimide ring compound II or either carbon of the CH₂ of ring opened compound III as depicted below:

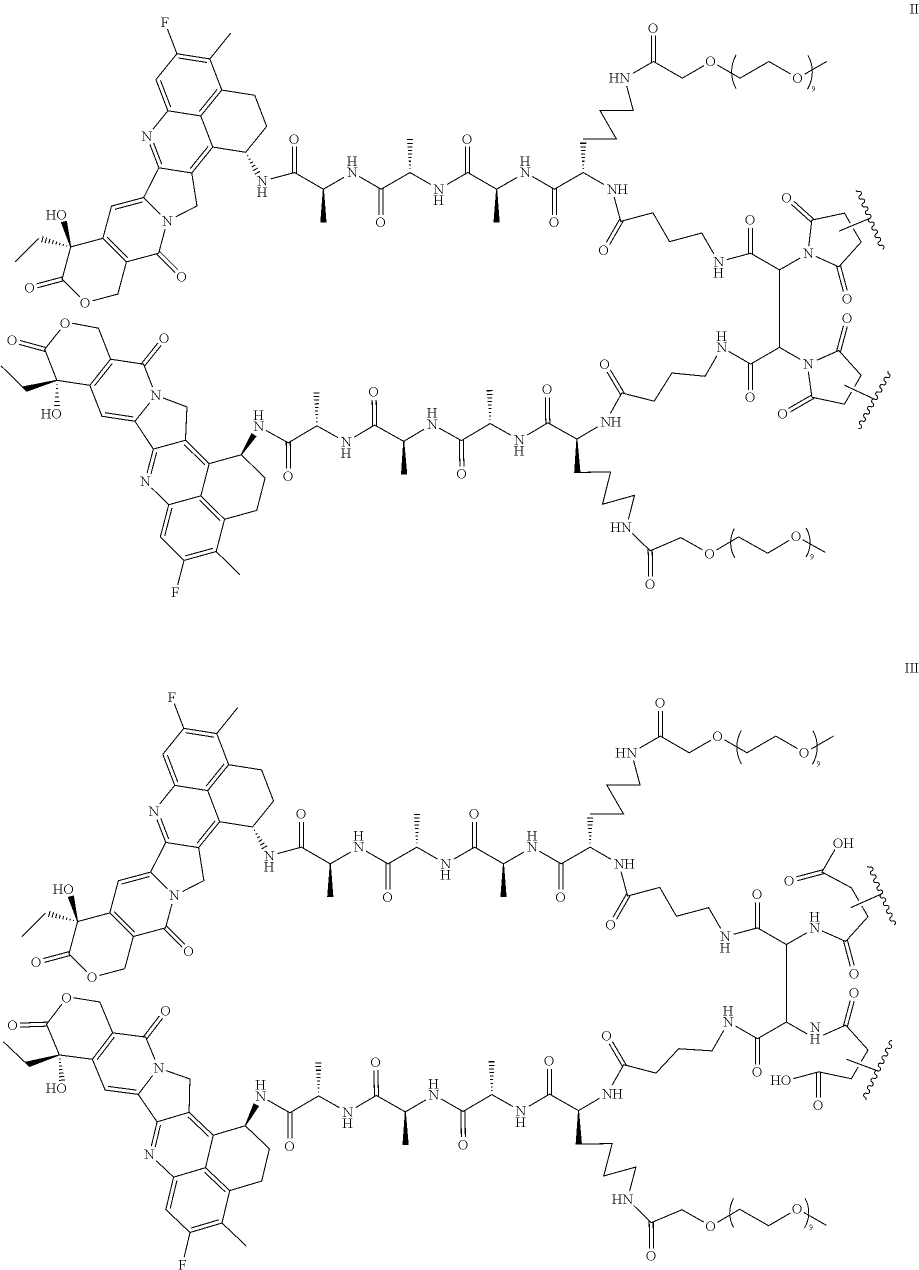

Disclosed herein are pharmaceutical compositions comprising any of the herein disclosed bispecific anti-EGFR/c-Met ADCs and a pharmaceutically acceptable excipient.

Disclosed herein are methods of treating an EGFR expressing cancer and/or c-Met expressing cancer in a patient in need thereof, comprising administering any of the herein disclosed bispecific anti-EGFR/c-Met ADCs or any of the herein disclosed pharmaceutical compositions to the patient to thereby treat the cancer.

### BRIEF DESCRIPTION OF THE DRAWINGS

The summary, as well as the following detailed description, is further understood when read in conjunction with the appended drawings. For the purpose of illustrating the disclosed ADCs and methods, the drawings show exemplary embodiments of the ADCs and methods; however, the ADCs and methods are not limited to the specific embodiments disclosed herein. In the drawings:

FIG. 1 depicts an exemplary topoisomerase linker-payload (CPT-113) used in the herein disclosed bispecific anti-EGFR/c-Met antibody-drug conjugates.

FIG. 2 shows the antibody binding capacity of an exemplary bispecific anti-EGFR/c-Met antibody-drug conjugate to the indicated EGFR and MET expressing cells. Antibody Binding Capacity was calculated with Bang's Quick Cal v2.3 for cell lines: MIA Paca-2, PANC-1, AsPC-1, A549, H1975, HCT116, and SNU-5. Antibody Binding Capacity was calculated with Bang's Quick Cal v3.0 for cell lines: H358, EBC1, H1373, HCC827, Detroit562, FaDu, HT-29, HCT-15, and LoVo.

FIG. 3 shows the cellular binding of an exemplary bispecific anti-EGFR/c-Met antibody-drug conjugate to the indicated EGFR and MET expressing cells.

FIG. 4A illustrates internalization of amivantamab in HCC827 cells.

FIG. 4B shows internalization of amivantamab in HCC827 across antibody concentrations.

FIG. 4C illustrates internalization of Ab1-ADC in HCC827 cells.

FIG. 4D shows internalization of Ab1-ADC in indicated EGFR and MET expressing cells across Ab1-ADC concentrations.

FIG. 5A shows the cytotoxicity of an exemplary bispecific anti-EGFR/c-Met antibody-drug conjugate in HCC827 cells (EGFR AMP/Del 19).

FIG. 5B shows the cytotoxicity of an exemplary bispecific anti-EGFR/c-Met antibody-drug conjugate in EBC-1 cells (MET AMP).

FIG. 6 shows tumor growth inhibition of an exemplary bispecific anti-EGFR/c-Met antibody-drug conjugate in EBC-1 cells (MET AMP; Ami Insensitive).

FIG. 7 shows tumor growth inhibition of an exemplary bispecific anti-EGFR/c-Met antibody-drug conjugate in H1975 cells (EGFR L858R/T790M; Ami Sensitive).

FIG. 8 shows tumor growth inhibition of an exemplary bispecific anti-EGFR/c-Met antibody-drug conjugate in H1373 (KRAS G12C) cells (EGFR/MET High; Ami Insensitive).

FIG. 9 shows tumor growth inhibition of an exemplary bispecific anti-EGFR/c-Met antibody-drug conjugate in A549 (KRAS G12S) cells (EGFR/MET Low; Ami Insensitive).

FIG. 10 shows tumor growth inhibition of an exemplary bispecific anti-EGFR/c-Met antibody-drug conjugate in colorectal cancer tumor xenograft model (CR3262).

FIG. 11A depicts plasma concentrations of Ab1-ADC at DARs of 4, 6 and 8, and Ab1-ADC2 at DAR 8 dose at 6 mg/kg in non-tumor bearing nude mice using anti-Fc antibody R10 as the capture reagent.

FIG. 11B depicts plasma concentrations of Ab1-ADC at DARs of 4, 6 and 8, and Ab1-ADC2 at DAR 8 dose at 6 mg/kg in non-tumor bearing nude mice using cMET as the capture reagent.

### DETAILED DESCRIPTION OF ILLUSTRATIVE EMBODIMENTS

The disclosed ADCs and methods may be understood more readily by reference to the following detailed description taken in connection with the accompanying figures, which form a part of this disclosure. It is to be understood that the disclosed ADCs and methods are not limited to the specific ADCs and methods described and/or shown herein, and that the terminology used herein is for the purpose of describing particular embodiments by way of example only and is not intended to be limiting of the claimed ADCs and methods.

Unless specifically stated otherwise, any description as to a possible mechanism or mode of action or reason for improvement is meant to be illustrative only, and the disclosed ADCs and methods are not to be constrained by the correctness or incorrectness of any such suggested mechanism or mode of action or reason for improvement.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the herein disclosure. Where a range of numerical values is stated herein as being less than a stated value, the range is nevertheless bounded on its lower end by a non-zero value. It is not intended that the scope of the methods be limited to the specific values recited when defining a range. All ranges are inclusive and combinable.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “about” when used in reference to numerical ranges, cutoffs, or specific values is used to indicate that the recited values may vary by up to as much as 10% from the listed value. As many of the numerical values used herein are experimentally determined, it should be understood by those skilled in the art that such determinations can, and often times will, vary among different experiments. The values used herein should not be considered unduly limiting by virtue of this inherent variation. Thus, the term “about” is used to encompass variations of ±10% or less, variations of ±5% or less, variations of ±1% or less, variations of ±0.5% or less, or variations of ±0.1% or less from the specified value.

“Treat,” “treatment,” and like terms refer to therapeutic treatment, and includes reducing the severity and/or frequency of symptoms, eliminating symptoms and/or the underlying cause of the symptoms, reducing the frequency or likelihood of symptoms and/or their underlying cause, and improving or remediating damage caused, directly or indirectly, by a cancer.

As used herein, “administering to the patient” and similar terms indicate a procedure by which, for example, the bispecific anti-EGFR/c-Met ADC is injected into a patient such that target cells, tissues, or segments of the body of the subject are contacted with the bispecific anti-EGFR/c-Met ADC.

The term “subject” as used herein is intended to mean any animal, in particular, mammals. The methods are applicable to human and nonhuman animals, although preferably used with mice and humans, and most preferably with humans. “Subject” and “patient” are used interchangeably herein.

The term “antibody” is meant in a broad sense and includes full length immunoglobulin molecules and antigen-binding fragments thereof.

Immunoglobulins can be assigned to five major classes, namely IgA, IgD, IgE, IgG, and IgM, depending on the heavy chain constant domain amino acid sequence. IgA and IgG are further sub-classified as the isotypes IgA1, IgA2, IgG1, IgG2, IgG3, and IgG4. Antibody light chains of any vertebrate species can be assigned to one of two clearly distinct types, namely kappa (κ) and lambda (2), based on the amino acid sequences of their constant domains.

“Antigen-binding fragment” refers to a portion of an immunoglobulin molecule that retains the antigen binding properties of the parental full length antibody (i.e., “antigen-binding fragment thereof”). Exemplary antigen binding fragments can have: heavy chain complementarity determining regions (CDR) 1, 2, and/or 3; light chain CDR 1, 2, and/or 3; a heavy chain variable region (VH); a light chain variable region (VL); and combinations thereof. Antigen binding fragments include: a Fab fragment (a monovalent fragment consisting of the VL, VH, constant light (CL), and constant heavy 1 (CH₁) domains); a F (ab) 2 fragment (a bivalent fragment comprising two Fab fragments linked by a disulfide bridge at the hinge region); a Fd fragment consisting of the VH and CH₁ domains; a Fv fragment consisting of the VL and VH domains of a single arm of an antibody; and a domain antibody (dAb) fragment (Ward et al., Nature 341:544-546, 1989), which consists of a VH domain or a VL domain. VH and VL domains can be engineered and linked together via a synthetic linker to form various types of single chain antibody designs where the VH/VL domains pair intramolecularly, or intermolecularly in those cases when the VH and VL domains are expressed by separate single chain antibody constructs, to form a monovalent antigen binding site, such as single chain Fv (scFv) or diabody, described for example in International Publication. Nos. WO1998/44001, WO1988/01649, WO1994/13804, and WO1992/01047. These antibody fragments are obtained using techniques well known to those of skill in the art, and the fragments are screened for utility in the same manner as are full length antibodies.

An antibody variable region consists of four “framework” regions interrupted by three “antigen binding sites.” The antigen binding sites are defined using various terms: (i) Complementarity Determining Regions (CDRs), three in the VH (HCDR1, HCDR2, HCDR3), and three in the VL (LCDR1, LCDR2, LCDR3) are based on sequence variability (Wu and Kabat J Exp Med 132:211-50, 1970; Kabat et al., Sequences of Proteins of Immunological Interest, 5th Ed. Public Health Service, National Institutes of Health, Bethesda, Md., 1991); and (ii) “Hypervariable regions” (“HVR” or “HV”), three in the VH (H1, H2, H3) and three in the VL (L1, L2, L3) refer to the regions of the antibody variable domains which are hypervariable in structure as defined by Chothia and Lesk (Chothia and Lesk Mol Biol 196:901-17, 1987). The AbM definition of CDRs is also widely used; it is a compromise between Kabat and Chothia numbering schemes and is so-called because it was used by Oxford Molecular's AbM antibody modelling software (Rees, A. R., Searle, S. M. J., Henry, A. H. and Pedersen, J. T. (1996) In Sternberg M. J. E. (ed.), Protein Structure Prediction. Oxford University Press, Oxford, 141-172). Other terms include “IMGT-CDRs” (Lefranc et al., Dev Comparat Immunol 27:55-77, 2003) and “Specificity Determining Residue Usage” (SDRU) (Almagro Mol Recognit 17:132-43, 2004). The International ImMunoGeneTics (IMGT) database (www_imgt_org) provides a standardized numbering and definition of antigen-binding sites. The correspondence between CDRs, HVs and IMGT delineations is described in Lefranc et al., Dev Comparat Immunol 27:55-77, 2003.

“Framework” or “framework sequences” are the remaining sequences of a variable region other than those defined to be antigen binding sites. Because the antigen binding sites can be defined by various terms as described above, the exact amino acid sequence of a framework depends on how the antigen-binding site was defined.

The term “bispecific anti-EGFR/c-Met antibody” or “bispecific EGFR/c-Met antibody” as used herein refers to an antibody having a first domain that specifically binds EGFR (EGFR binding arm) and a second domain that specifically binds c-Met (c-Met binding arm). The domains specifically binding EGFR and c-Met are typically VH/VL pairs, and the bispecific anti-EGFR/c-Met antibody is monovalent in terms of binding to EGFR and c-Met. The term “bispecific anti-EGFR/c-Met antibody” also includes antigen-binding fragments of the EGFR binding arm, antigen-binding fragments of the c-Met binding arm, or antigen-binding fragments of the EGFR binding arm and antigen-binding fragments of the c-Met binding arm.

“Amivantamab” is a fully humanized IgG1-based bispecific antibody directed against EGFR and c-MET, described in U.S. Pat. No. 9,593,164, which is incorporated herein by reference in its entirety. Amivantamab comprises a first domain that specifically binds EGFR (EGFR binding arm) that comprises a first heavy chain (HC1) comprising a heavy chain complementarity determining region 1 (HCDR1) comprising the amino acid sequence of SEQ ID NO: 1, a HCDR2 comprising the amino acid sequence of SEQ ID NO: 2, and a HCDR3 comprising the amino acid sequence of SEQ ID NO: 3; a first light chain (LC1) comprising a light chain complementarity determining region 1 (LCDR1) comprising the amino acid sequence of SEQ ID NO: 4, a LCDR2 comprising the amino acid sequence of SEQ ID NO: 5, and a LCDR3 comprising the amino acid sequence of SEQ ID NO: 6; a second heavy chain (HC2) comprising a HCDR1 comprising the amino acid sequence of SEQ ID NO: 7, a HCDR2 comprising the amino acid sequence of SEQ ID NO: 8, and a HCDR3 comprising the amino acid sequence of SEQ ID NO: 9; and a second light chain (LC2) comprising a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12. In some embodiments, amivantamab comprises a first domain that specifically binds EGFR (EGFR binding arm) that comprises a first heavy chain (HC1) comprising a variable region (VH1) comprising the amino acid sequence of SEQ ID NO: 13, a first light chain (LC1) comprising a variable region (VL1) comprising the amino acid sequence of SEQ ID NO: 14, a HC2 comprising a variable region (VH2) comprising the amino acid sequence of SEQ ID NO: 15, and a LC2 comprising a variable region (VL2) comprising the amino acid sequence of SEQ ID NO: 16. In some embodiments, amivantamab comprises a first domain that specifically binds EGFR (EGFR binding arm) that comprises a first heavy chain (HC1) comprising the amino acid sequence of SEQ ID NO: 17 and a first light chain (LC1) comprising the amino acid sequence of SEQ ID NO: 18, and a second domain that specifically binds c-Met (c-Met binding arm) that comprises a heavy chain (HC2) comprising the amino acid sequence of SEQ ID NO: 19 and a light chain (LC2) comprising the amino acid sequence of SEQ ID NO: 20. In some embodiments, amivantamab comprises a biosimilar thereof.

It is to be appreciated that certain features of the disclosed ADCs and methods which are, for clarity, described herein in the context of separate embodiments, may also be provided in combination in a single embodiment. Conversely, various features of the disclosed ADCs and methods that are, for brevity, described in the context of a single embodiment, may also be provided separately or in any subcombination.

As used herein, the singular forms “a,” “an,” and “the” include the plural.

When a list is presented, unless stated otherwise, it is to be understood that each individual element of that list, and every combination of that list, is a separate embodiment. For example, a list of embodiments presented as “A, B, or C” or “A, B, and/or C” is to be interpreted as including the embodiments: “A;” “B;” “C;” “A or B;” “A or C;” “B or C;” or “A, B, or C.”

The term “comprising” is intended to include examples encompassed by the terms “consisting essentially of” and “consisting of”; similarly, the term “consisting essentially of” is intended to include examples encompassed by the term “consisting of.”

In chemical structures depicted herein, a line crossing through a bond between two neighboring carbon atoms means the line represents a bond with either of the two neighboring carbon atoms.

Disclosed herein are bispecific anti-EGFR/c-Met antibody-drug conjugates (ADCs), comprising a bispecific anti-EGFR/c-Met antibody that comprises
a first heavy chain (HC1) comprising a heavy chain complementarity determining region 1 (HCDR1) comprising the amino acid sequence of SEQ ID NO: 1, a HCDR2 comprising the amino acid sequence of SEQ ID NO: 2, and a HCDR3 comprising the amino acid sequence of SEQ ID NO: 3;
a first light chain (LC1) comprising a light chain complementarity determining region 1 (LCDR1) comprising the amino acid sequence of SEQ ID NO: 4, a LCDR2 comprising the amino acid sequence of SEQ ID NO: 5, and a LCDR3 comprising the amino acid sequence of SEQ ID NO: 6;
a second heavy chain (HC2) comprising a HCDR1 comprising the amino acid sequence of SEQ ID NO: 7, a HCDR2 comprising the amino acid sequence of SEQ ID NO: 8, and a HCDR3 comprising the amino acid sequence of SEQ ID NO: 9; and
a second light chain (LC2) comprising a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12.

The ADCs also comprise a dual maleimide group having the structure:

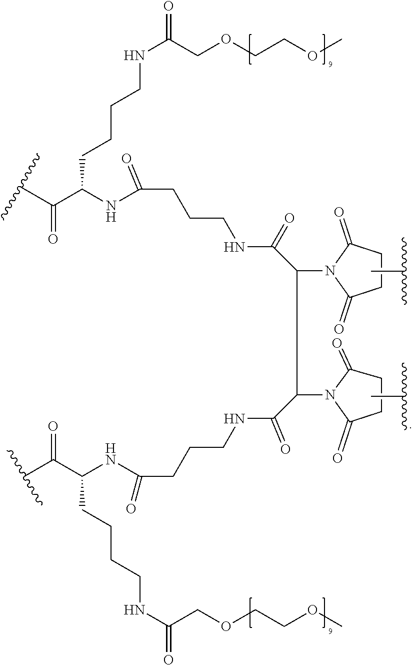

The bispecific anti-EGFR/c-Met antibody may be connected to the dual maleimide group through one carbon atom in each maleimide. In some embodiments, each maleimide may attach to the bispecific anti-EGFR/c-Met antibody at either carbon of the relevant CH₂ group of the 5-membered ring.

The bispecific anti-EGFR/c-Met antibody may be connected or conjugated to the dual maleimide group through one carbon atom in each maleimide. In some embodiments, each maleimide may attach to the bispecific anti-EGFR/c-Met antibody at either carbon of the relevant CH₂ group of the 5-membered ring. In other embodiments, said connection or conjugation is at one or more native or introduced cysteine residues of said antibody. In other embodiments, said connection or conjugation is at one or more interchain cysteine residues of said antibody. In other embodiments, said connection or conjugation is at one or more interchain cysteine residues making up the HC-LC disulfide bonds of said antibody. In other embodiments, said connection or conjugation is limited at cysteine residues making up the hinge disulfide bonds of said antibody. In other embodiments, said connection or conjugation is not at cysteine residues making up the hinge disulfide bonds of said antibody. In other embodiments, said connection or conjugation is at the interchain cysteine residues making up the HC-LC disulfide bonds of said antibody wherein said connection or conjugation results in a DAR of 2 to 8. In other embodiments, said connection or conjugation is at the interchain cysteine residues making up the HC-LC disulfide bonds of said antibody wherein said connection or conjugation results in a DAR of about 4.

The ADCs further comprise an enzymatically cleavable tri-alanine linker having the structure:

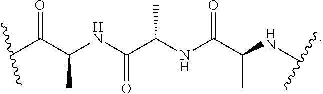 [SMILES: *N[C@@H](C)C(=O)N[C@@H](C)C(=O)N[C@@H](C)C(*)=O]

In some embodiments, the ADCs contain one tri-alanine linker. In some embodiments, ADCs comprise two tri-alanine linkers.

The ADCs also comprise a topoisomerase I inhibitor moiety having the structure:

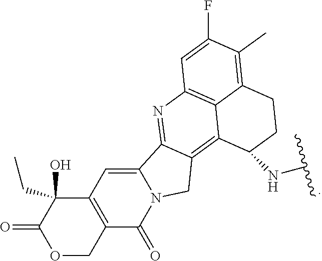 [SMILES: *N[C@H]1CCc2c(C)c(F)cc3nc4c(c1c23)Cn1c-4cc2c(c1=O)COC(=O)[C@]2(O)CC]

In some embodiments, the ADCs contain one topoisomerase I moiety. In other embodiments, the ADCs comprise two topoisomerase I moieties.

In chemical structures depicted herein, a line crossing through a bond between two neighboring carbon atoms means the line represents a bond with either of the two neighboring carbon atoms.

According to the disclosure, the ADCs comprise the dual maleimide group, the tri-alanine linker, and the topoisomerase I inhibitor moiety. In some embodiments, the dual maleimide group is bound to the tri-alanine linker. In some embodiments, the tri-alanine linker is bound to the topoisomerase I inhibitor moiety. In some embodiments, the ADCs comprise a compound that is (dual maleimide group)-(tri-alanine linker)-(topoisomerase I inhibitor moiety). In some embodiments, the ADCs comprise linker-payload (compound I (CPT-113)):

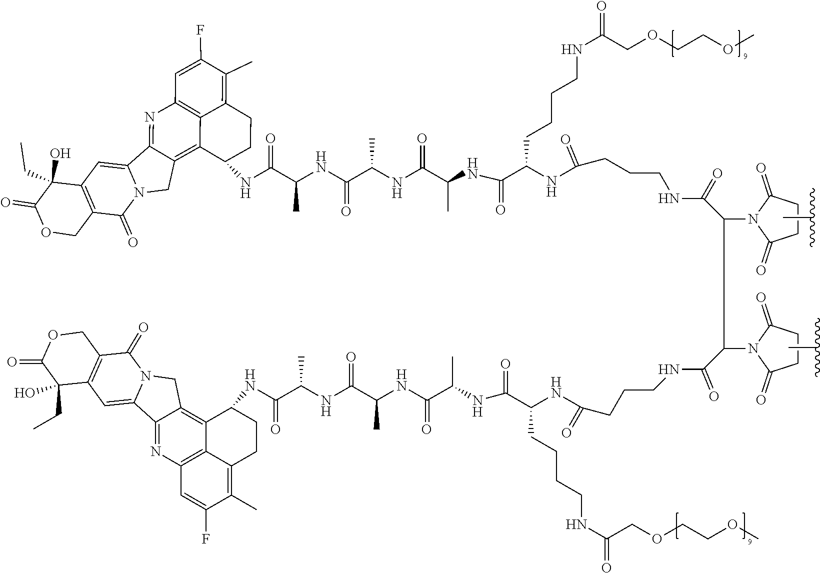

### I.

In some embodiments the ADC is Ab1-ADC comprises the following structure of Formula I-A:

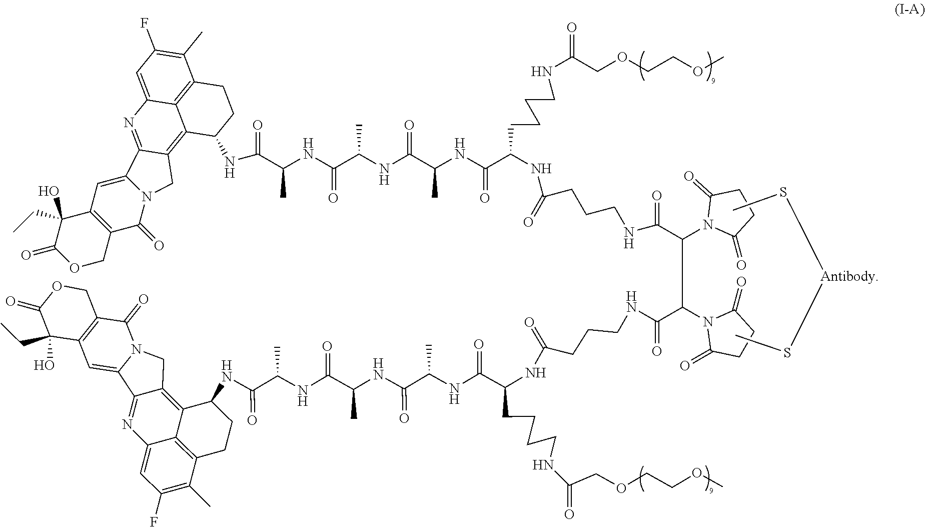

In some embodiments the ADC is Ab1-ADC (bismaleimide conjugation moiety) comprises the following structure of Formula II-A:

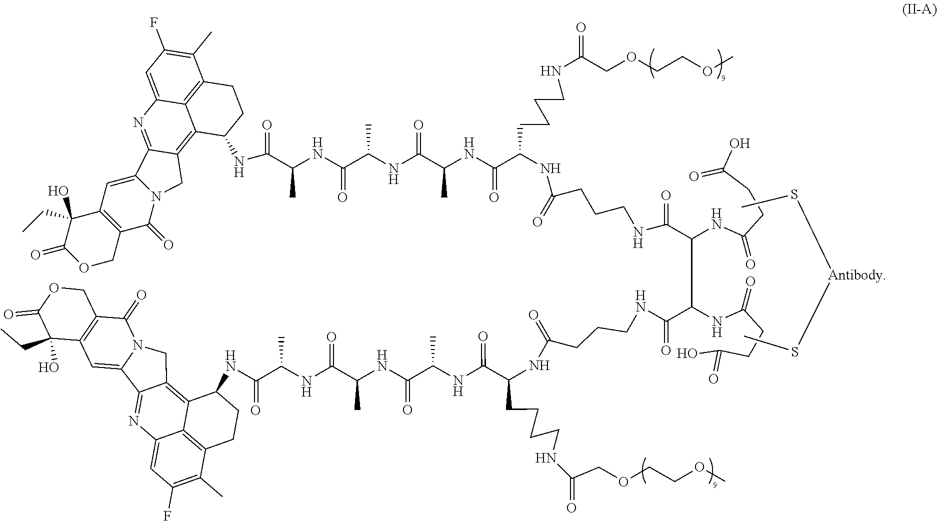

According to the disclosure, the ADCs may also comprise a single maleimide group, the tri-alanine linker, and the topoisomerase I inhibitor moiety.

In some embodiments the ADCs also comprise a single maleimide group having the structure:

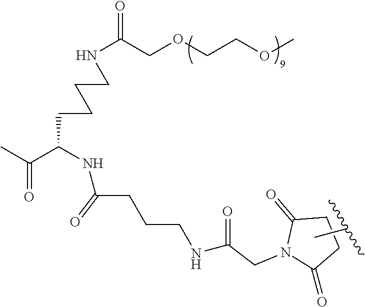

The ADCs further comprise a tri-alanine linker having the structure:

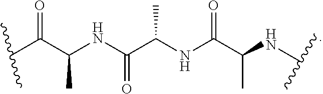 [SMILES: *N[C@@H](C)C(=O)N[C@@H](C)C(=O)N[C@@H](C)C(*)=O]

The ADCs also comprise a topoisomerase I inhibitor moiety having the structure:

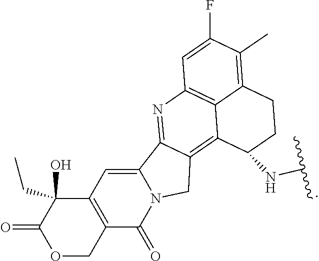 [SMILES: *N[C@H]1CCc2c(C)c(F)cc3nc4c(c1c23)Cn1c-4cc2c(c1=O)COC(=O)[C@]2(O)CC]

In some embodiments, the ADCs comprise a compound that is (single maleimide group)-(tri-alanine linker)-(topoisomerase I inhibitor moiety). In some embodiments, the ADCs comprise compound II:

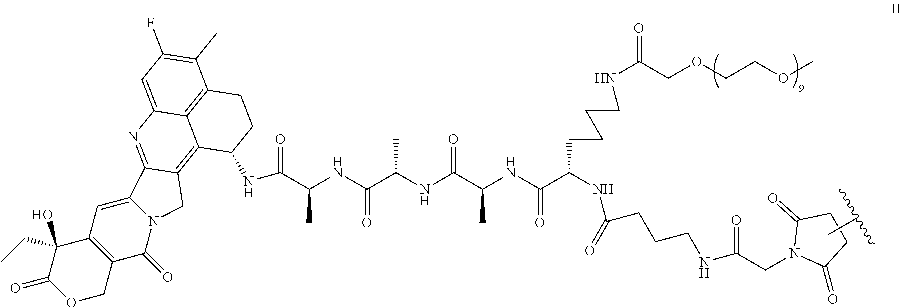

In some embodiments the ADC is Ab1-ADC2 comprising the following structure:

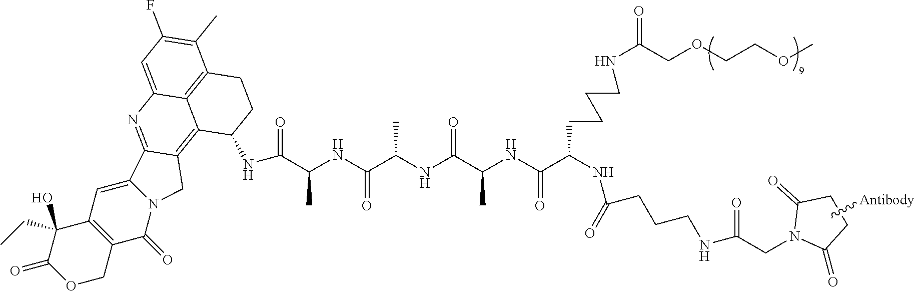

The bispecific anti-EGFR/c-Met antibody may be connected to the single maleimide group through one carbon atom in the maleimide. In some embodiments, the maleimide may attach to the bispecific anti-EGFR/c-Met antibody at either carbon of the relevant CH₂ group of the 5-membered ring.

The bispecific anti-EGFR/c-Met ADCs can comprise a bispecific anti-EGFR/c-Met antibody comprising a HC1 comprising a variable region (VH1) comprising the amino acid sequence of SEQ ID NO: 13, a LC1 comprising a variable region (VL1) comprising the amino acid sequence of SEQ ID NO: 14, a HC2 comprising a variable region (VH2) comprising the amino acid sequence of SEQ ID NO: 15, and a LC2 comprising a variable region (VL2) comprising the amino acid sequence of SEQ ID NO: 16.

The bispecific anti-EGFR/c-Met ADCs can comprise a bispecific anti-EGFR/c-Met antibody that is an IgG1 isotype.

The bispecific anti-EGFR/c-Met ADCs can comprise a bispecific anti-EGFR/c-Met antibody comprising a HC1 comprising the amino acid sequence of SEQ ID NO: 17, a LC1 comprising the amino acid sequence of SEQ ID NO: 18, a HC2 comprising the amino acid sequence of SEQ ID NO: 19, and a LC2 comprising the amino acid sequence of SEQ ID NO: 20.

The bispecific anti-EGFR/c-Met ADCs can comprise a bispecific anti-EGFR/c-Met antibody comprising a biantennary glycan structure with a fucose content between 0% to 20%.

“Fucose content” refers to the amount of the fucose monosaccharide within the sugar chain at Asn297. The relative amount of fucose is the percentage of fucose-containing structures related to all glycostructures. These may be characterized and quantified by multiple methods, for example: 1) using MALDI-TOF of N-glycosidase F treated sample (e.g., complex, hybrid and oligo- and high-mannose structures) as described in Int Pub. No. WO2008/077546 2); 2) by enxymatic release of the Asn297 glycans with subsequent derivatization and detection/quantitation by HPLC (UPLC) with fluorescence detection and/or HPLC-MS (UPLC-MS); 3) intact protein analysis of the native or reduced mAb, with or without treatment of the Asn297 glycans with Endo S or other enzyme that cleaves between the first and the second GlcNAc monosaccharides, leaving the fucose attached to the first GlcNAc; 4) digestion of the mAb to constituent peptides by enzymatic digestion (e.g., trypsin or endopeptidase Lys-C), and subsequent separation, detection and quantitation by HPLC-MS (UPLC-MS); 5) Separation of the mAb oligosaccharides from the mAb protein by specific enzymatic deglycosylation with PNGase F at Asn 297. The oligosaccharides thus released can be labeled with a fluorophore, separated and identified by various complementary techniques which allow: fine characterization of the glycan structures by matrix-assisted laser desorption ionization (MALDI) mass spectrometry by comparison of the experimental masses with the theoretical masses, determination of the degree of sialylation by ion exchange HPLC (GlycoSep C), separation and quantification of the oligosaccharide forms according to hydrophilicity criteria by normal-phase HPLC (GlycoSep N), and separation and quantification of the oligosaccharides by high performance capillary electrophoresis-laser induced fluorescence (HPCE-LIF).

In some embodiments, the anti-EGFR/c-Met ADC comprises a bispecific anti-EGFR/c-Met antibody comprising a low fucose content. “Low fucose” or “low fucose content” as used in the application refers to antibodies with fucose content of about 0%-about 20%. The bispecific anti-EGFR/c-Met antibody can comprise a fucose content of about 20%, about 19%, about 18%, about 17%, about 16%, about 15%, about 14%, about 13%, about 12%, about 11% about 10%, about 9%, about 8%, about 7%, about 6%, about 5%, about 4%, about 3%, about 2%, or about 1%.

In some embodiments, the bispecific anti-EGFR/c-Met ADCs comprise a bispecific anti-EGFR/c-Met antibody comprising normal levels of fucose. “Normal levels of fucose” or “normal fucose content” as used herein refers to antibodies with fucose content of about over 50%, typically about over 80% or over 85%.

In some embodiments, the bispecific anti-EGFR/c-Met ADCs comprise a bispecific anti-EGFR/c-Met antibody comprising a fucose content of over about 20%. In some embodiments, the fucose content is from about 20% to about 50%.

In some embodiments, the bispecific anti-EGFR/c-Met ADCs can comprise a bispecific anti-EGFR/c-Met antibody that is amivantamab.

The bispecific anti-EGFR/c-Met antibody may be connected or conjugated to the dual maleimide group through one carbon atom in each maleimide. In some embodiments, each maleimide may attach to the bispecific anti-EGFR/c-Met antibody at either carbon of the relevant CH₂ group of the 5-membered ring. In other embodiments, said connection or conjugation is at one or more native or introduced cysteine residues of said antibody. In other embodiments, said connection or conjugation is at one or more interchain cysteine residues of said antibody. In other embodiments, said connection or conjugation is at one or more interchain cysteine residues making up the HC-LC disulfide bond of said antibody. In other embodiments, said connection or conjugation is limited at cysteine residues making up the hinge disulfide bonds of said antibody. In other embodiments, said connection or conjugation is not at cysteine residues making up the hinge disulfide bonds of said antibody. In other embodiments, said connection or conjugation is at the interchain cysteine residues making up the HC-LC disulfide bonds of said antibody wherein said connection or conjugation results in a DAR of 2 to 8. In other embodiments, said connection or conjugation is at the interchain cysteine residues making up the HC-LC disulfide bonds of said antibody wherein said connection or conjugation results in a DAR of about 4.

In some embodiments, the bispecific anti-EGFR/c-Met ADCs can have a drug-to-antibody ratio (DAR) of about 2 to 8.

In some embodiments, the bispecific anti-EGFR/c-Met ADCs can have DAR of about 4.

In some embodiments, the topoisomerase I inhibitor moiety (CPT-116) comprises the structure:

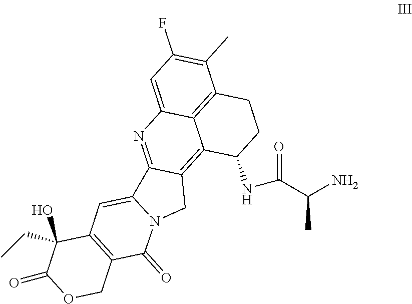 [SMILES: CC[C@@]1(O)C(=O)OCc2c1cc1n(c2=O)Cc2c-1nc1cc(F)c(C)c3c1c2[C@@H](NC(=O)[C@H](C)N)CC3]

Disclosed herein are pharmaceutical compositions comprising any of the herein disclosed bispecific anti-EGFR/c-Met ADCs and a pharmaceutically acceptable excipient. Such pharmaceutical compositions comprise the non-hydrolyzed form of the ADC, the hydrolyzed form of the ADC, or both the non-hydrolyzed form and hydrolyzed form of the ADC. For example, pharmaceutical compositions disclosed herein comprise the ADC of Formula I-A, Formula II-A, or Formula I-A and II-A.

Disclosed herein are methods of treating an EGFR-expressing cancer and/or c-Met-expressing cancer in a patient in need thereof, the methods comprising administering any of the herein disclosed bispecific anti-EGFR/c-Met ADCs or any of the herein disclosed pharmaceutical compositions to the patient to thereby treat the cancer. In some embodiments, the methods comprise administering to the patient any of the herein disclosed bispecific anti-EGFR/c-Met ADCs. In some embodiments, the methods comprise administering to the patient any of the herein disclosed pharmaceutical compositions.

Suitable cancers include, for example, lung cancer, colorectal cancer, and head and neck cancer. In some embodiments, the cancer is lung cancer. In some embodiments, the cancer is colorectal cancer. In some embodiments, the cancer is head and neck cancer.

In some embodiments, the lung cancer is epithelial lung cancer. In some embodiments, the lung cancer is non-small cell lung cancer (NSCLC). The NSCLC can be squamous cell carcinoma, large cell carcinoma, or adenocarcinoma. In some embodiments, the NSCLC is squamous cell carcinoma. In some embodiments, the NSCLC is large cell carcinoma. In some embodiments, the NSCLC is adenocarcinoma.

SEQUENCES

>SEQ ID NO: 1 (HCDR1, EGFR binding arm)
TYGMH
>SEQ ID NO: 2 (HCDR2, EGFR binding arm)
VIWDDGSYKYYGDSVKG
>SEQ ID NO: 3 (HCDR3, EGFR binding arm)
DGITMVRGVMKDYFDY
>SEQ ID NO: 4 (LCDR1, EGFR binding arm)
RASQDISSALV
>SEQ ID NO: 5 (LCDR2, EGFR binding arm)
DASSLES
>SEQ ID NO: 6 (LCDR3, EGFR binding arm)
QQFNSYPLT
>SEQ ID NO: 7 (HCDR1, c-Met binding arm)
SYGIS
>SEQ ID NO: 8 (HCDR2, c-Met binding arm)
WISAYNGYTNYAQKLQG
>SEQ ID NO: 9 (HCDR3, c-Met binding arm)
DLRGTNYFDY
>SEQ ID NO: 10 (LCDR1, c-Met binding arm)
RASQGISNWLA
>SEQ ID NO: 11 (LCDR2, c-Met binding arm)
AASSLLS
>SEQ ID NO: 12 (LCDR3, c-Met binding arm)
QQANSFPIT
>SEQ ID NO: 13 (VH, EGFR binding arm)
QVQLVESGGGVVQPGRSLRLSCAASGFTFSTYGMHWVRQAPGKGLEWVAVIWDDGSYK
YYGDSVKGRFTISRDNSKNTLYLQMNSLRAEDTAVYYCARDGITMVRGVMKDYFDYWG
QGTLVTVSS
>SEQ ID NO: 14 (VL, EGFR binding arm)
AIQLTQSPSSLSASVGDRVTITCRASQDISSALVWYQQKPGKAPKLLIYDASSLESGVPSRFS
GSESGTDFTLTISSLQPEDFATYYCQQFNSYPLTFGGGTKVEIK
>SEQ ID NO: 15 (VH, c-Met binding arm)
QVQLVQSGAEVKKPGASVKVSCETSGYTFTSYGISWVRQAPGHGLEWMGWISAYNGYTN
YAQKLQGRVTMTTDTSTSTAYMELRSLRSDDTAVYYCARDLRGTNYFDYWGQGTLVTVS
S
>SEQ ID NO: 16 (VL, c-Met binding arm)
DIQMTQSPSSVSASVGDRVTITCRASQGISNWLAWFQHKPGKAPKLLIYAASSLLSGVPSRF
SGSGSGTDFTLTISSLQPEDFATYYCQQANSFPITFGQGTRLEIK
>SEQ ID NO: 17 (HC1, EGFR binding arm)
QVQLVESGGGVVQPGRSLRLSCAASGFTFSTYGMHWVRQAPGKGLEWVAVIWDDGSY
KYYGDSVKGRFTISRDNSKNTLYLQMNSLRAEDTAVYYCARDGITMVRGVMKDYFDY
WGQGTLVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKRVEPKSCDKTHT
CPPCPAPELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSD
GSFLLYSKLTVDKSRWQQGNVFSCSVMHEALHNHYTQKSLSLSPGK
>SEQ ID NO: 18 (LC1, EGFR binding arm)
AIQLTQSPSSLSASVGDRVTITCRASQDISSALVWYQQKPGKAPKLLIYDASSLESGVPSR
FSGSESGTDFTLTISSLQPEDFATYYCQQFNSYPLTFGGGTKVEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
>SEQ ID NO: 19 (HC2, c-Met binding arm)
QVQLVQSGAEVKKPGASVKVSCETSGYTFTSYGISWVRQAPGHGLEWMGWISAYNGY
TNYAQKLQGRVTMTTDTSTSTAYMELRSLRSDDTAVYYCARDLRGTNYFDYWGQGTL
VTVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKRVEPKSCDKTHTCPPCPAP
ELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSR
LTVDKSRWQQGNVFSCSVMHEALHNHYTQKSLSLSPGK
>SEQ ID NO: 20 (LC2, c-Met binding arm)
DIQMTQSPSSVSASVGDRVTITCRASQGISNWLAWFQHKPGKAPKLLIYAASSLLSGVPS
RFSGSGSGTDFTLTISSLQPEDFATYYCQQANSFPITFGQGTRLEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC

In some embodiments, anti-EGFR/c-Met antibodies disclosed herein comprise the CDR sequences shown in Table 1.

TABLE 1
CDR sequences of anti-EGFR/c-Met antibodies, according to indicated CDR
definitions.

| ID | EGFR binding Arm | | c-Met binding arm | |
|---|---|---|---|---|
| HC CDR1 (AbM) | GFTFSTYGMH | SEQ ID No: 21 | GYTFTSYGIS | SEQ ID No: 45 |
| HC CDR2 (AbM) | VIWDDGSYKY | SEQ ID No: 22 | WISAYNGYTN | SEQ ID No: 46 |
| HC CDR3 (AbM) | DGITMVRGVMKDYFDY | SEQ ID No: 23 | DLRGTNYFDY | SEQ ID No: 47 |
| LC CDR1 (AbM) | RASQDISSALV | SEQ ID No: 24 | RASQGISNWLA | SEQ ID No: 48 |
| LC CDR2 (AbM) | DASSLES | SEQ ID No: 25 | AASSLLS | SEQ ID No: 49 |
| LC CDR3 (AbM) | QQFNSYPLT | SEQ ID No: 26 | QQANSFPIT | SEQ ID No: 50 |
| HC CDR1 (CHOTHIA) | GFTFSTY | SEQ ID No: 27 | GYTFTSY | SEQ ID No: 51 |
| HC CDR2 (CHOTHIA) | WDDGSY | SEQ ID No: 28 | SAYNGY | SEQ ID No: 52 |
| HC CDR3 (CHOTHIA) | DGITMVRGVMKDYFDY | SEQ ID No: 29 | DLRGTNYFDY | SEQ ID No: 53 |
| LC CDR1 (CHOTHIA) | RASQDISSALV | SEQ ID No: 30 | RASQGISNWLA | SEQ ID No: 54 |
| LC CDR2 (CHOTHIA) | DASSLES | SEQ ID No: 31 | AASSLLS | SEQ ID No: 55 |
| LC CDR3 (CHOTHIA) | QQFNSYPLT | SEQ ID No: 32 | QQANSFPIT | SEQ ID No: 56 |
| HC CDR1 (IMGT) | GFTFSTYG | SEQ ID No: 33 | GYTFTSYG | SEQ ID No: 57 |
| HC CDR2 (IMGT) | IWDDGSYK | SEQ ID No: 34 | ISAYNGYT | SEQ ID No: 58 |
| HC CDR3 (IMGT) | ARDGITMVRGVMKDYFDY | SEQ ID No: 35 | ARDLRGTNYFDY | SEQ ID No: 59 |
| LC CDR1 (IMGT) | QDISSA | SEQ ID No: 36 | QGISNW | SEQ ID No: 60 |
| LC CDR2 (IMGT) | DAS | | AAS | |
| LC CDR3 (IMGT) | QQFNSYPLT | SEQ ID No: 38 | QQANSFPIT | SEQ ID No: 62 |
| HC CDR1 (CONTACT) | STYGMH | SEQ ID No: 39 | TSYGIS | SEQ ID No: 63 |
| HC CDR2 (CONTACT) | WVAVIWDDGSYKY | SEQ ID No: 40 | WMGWISAYNGYTN | SEQ ID No: 64 |
| HC CDR3 (CONTACT) | ARDGITMVRGVMKDYFD | SEQ ID No: 41 | ARDLRGTNYFD | SEQ ID No: 65 |
| LC CDR1 (CONTACT) | SSALVWY | SEQ ID No: 42 | SNWLAWF | SEQ ID No: 66 |
| LC CDR2 (CONTACT) | LLIYDASSLE | SEQ ID No: 43 | LLIYAASSLL | SEQ ID No: 67 |
| LC CDR3 (CONTACT) | QQFNSYPL | SEQ ID No: 44 | QQANSFPI | SEQ ID No: 68 |

### Generating Anti-EGFR/c-Met Antibodies

Anti-EGFR/c-Met antibodies used in the methods of the disclosure may be generated, for example, using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy chain disulfide bonds in the hinge regions of the parental monospecific antibodies are reduced. The resulting free cysteines of one of the parental monospecific antibodies form an inter heavy-chain disulfide bond with cysteine residues of a second parental monospecific antibody molecule and simultaneously CH3 domains of the parental antibodies release and reform by dissociation-association. The CH3 domains of the Fab arms may be engineered to favor heterodimerization over homodimerization. The resulting product is a bispecific antibody having two Fab arms or half molecules which each bind a distinct epitope, i.e., an epitope on EGFR and an epitope on c-Met. For example, the bispecific antibodies of the invention may be generated using the technology described in Int. Pat. Publ. No. WO2011/131746, which is incorporated herein by reference in its entirety. Mutations F405L in one heavy chain and K409R in the other heavy chain may be used in case of IgG1 antibodies. For IgG2 antibodies, a wild-type IgG2 and a IgG2 antibody with F405L and R409K substitutions may be used. For IgG4 antibodies, a wild-type IgG4 and a IgG4 antibody with F405L and R409K substitutions may be used. To generate bispecific antibodies, the first monospecific bivalent antibody and the second monospecific bivalent antibody are engineered to have the aforementioned mutation in the Fc region, and the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

Bispecific anti-EGFR/c-Met antibodies used in the methods of the disclosure may also be generated using other heterodimerization motifs such as Knob-in-Hole (Genentech), CrossMAbs (Roche) and electrostatically-matched (Chugai, Amgen, NovoNordisk, Oncomed), the LUZ-Y (Genentech), Strand Exchange Engineered Domain body (SEEDbody) (EMD Serono), and Biclonic (Merus).

In the “knob-in-hole” strategy (see, e.g., Intl. Publ. No. WO 2006/028936) select amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

CrossMAb technology, in addition to utilizing the “knob-in-hole” strategy to promote Fab arm exchange utilizes CH1/CL domain swaps in one half arm to ensure correct light chain pairing of the resulting bispecific antibody (see e.g., U.S. Pat. No. 8,242,247).

Other cross-over strategies may be used to generate full length bispecific antibodies of the invention by exchanging variable or constant, or both domains between the heavy chain and the light chain or within the heavy chain in the bispecific antibodies, either in one or both arms. These exchanges include for example VH-CH1 with VL-CL, VH with VL, CH3 with CL and CH3 with CH1 as described in Int. Patent Publ. Nos. WO2009/080254, WO2009/080251, WO2009/018386 and WO2009/080252.

Other strategies such as promoting heavy chain heterodimerization using electrostatic interactions by substituting positively charged residues at one CH3 surface and negatively charged residues at a second CH3 surface may be used, as described in US Patent Publ. No. US2010/0015133; US Patent Publ. No. US2009/0182127; US Patent Publ. No. US2010/028637 or US Patent Publ. No. US2011/0123532. In other strategies, heterodimerization may be promoted by the following substitutions (expressed as modified positions in the first CH₃ domain of the first heavy chain/modified position in the second CH₃ domain of the second heavy chain): L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in U.S. Patent Publ. No. US2012/0149876 or U.S. Patent Publ. No. US2013/0195849.

SEEDbody technology may be utilized to generate bispecific antibodies of the invention. SEEDbodies have, in their constant domains, select IgG residues substituted with IgA residues to promote heterodimerization as described in U.S. Patent No. US20070287170.

Mutations are typically made at the DNA level to a molecule such as the constant domain of the antibody using standard methods.

### ENUMERATED EMBODIMENTS

Non-limiting, enumerated embodiments are provided below.

Embodiment 1. A bispecific anti-EGFR/c-Met antibody-drug conjugate (ADC), comprising
a bispecific anti-EGFR/c-Met antibody that comprises:
   a first heavy chain (HC1) comprising a heavy chain complementarity determining region 1 (HCDR1) comprising the amino acid sequence of SEQ ID NO: 1, a HCDR2 comprising the amino acid sequence of SEQ ID NO: 2, and a HCDR3 comprising the amino acid sequence of SEQ ID NO: 3;
   a first light chain (LC1) comprising a light chain complementarity determining region 1 (LCDR1) comprising the amino acid sequence of SEQ ID NO: 4, a LCDR2 comprising the amino acid sequence of SEQ ID NO: 5, and a LCDR3 comprising the amino acid sequence of SEQ ID NO: 6;
   a second heavy chain (HC2) comprising a HCDR1 comprising the amino acid sequence of SEQ ID NO: 7, a HCDR2 comprising the amino acid sequence of SEQ ID NO: 8, and a HCDR3 comprising the amino acid sequence of SEQ ID NO: 9; and
   a second light chain (LC2) comprising a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12; and
compound I linker-payload (CPT-113)) that comprises:

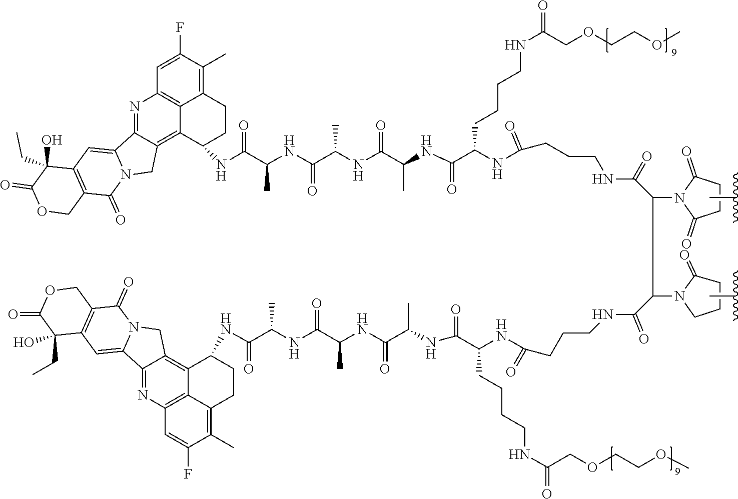

a dual maleimide group having the structure:

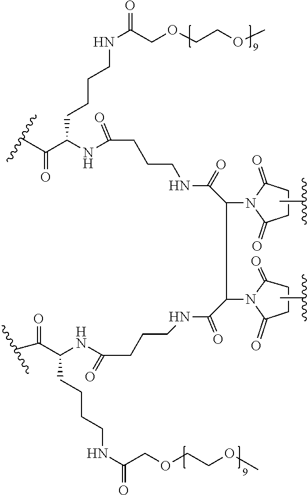

a tri-alanine linker having the structure:

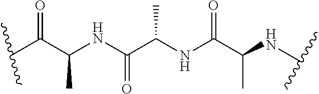 [SMILES: *N[C@@H](C)C(=O)N[C@@H](C)C(=O)N[C@@H](C)C(*)=O]

and a topoisomerase I inhibitor moiety having the structure:

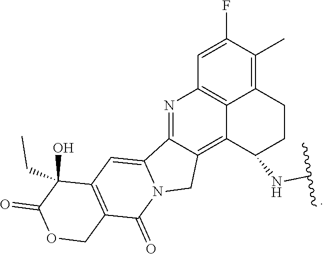 [SMILES: *N[C@H]1CCc2c(C)c(F)cc3nc4c(c1c23)Cn1c-4cc2c(c1=O)COC(=O)[C@]2(O)CC]

Embodiment 2. The bispecific anti-EGFR/c-Met ADC of embodiment 1, comprising two tri-alanine linkers.

Embodiment 3. The bispecific anti-EGFR/c-Met ADC of embodiment 1 or 2, comprising two topoisomerase I moieties.

Embodiment 4. A bispecific anti-EGFR/c-Met antibody-drug conjugate (ADC), comprising:
a bispecific anti-EGFR/c-Met antibody that comprises:
   a first heavy chain (HC1) comprising a heavy chain complementarity determining region 1 (HCDR1) comprising the amino acid sequence of SEQ ID NO: 1, a HCDR2 comprising the amino acid sequence of SEQ ID NO: 2, and a HCDR3 comprising the amino acid sequence of SEQ ID NO: 3;
   a first light chain (LC1) comprising a light chain complementarity determining region 1 (LCDR1) comprising the amino acid sequence of SEQ ID NO: 4, a LCDR2 comprising the amino acid sequence of SEQ ID NO: 5, and a LCDR3 comprising the amino acid sequence of SEQ ID NO: 6;
   a second heavy chain (HC2) comprising a HCDR1 comprising the amino acid sequence of SEQ ID NO: 7, a HCDR2 comprising the amino acid sequence of SEQ ID NO: 8, and a HCDR3 comprising the amino acid sequence of SEQ ID NO: 9; and
   a second light chain (LC2) comprising a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12; and wherein the bispecific anti-EGFR/c-Met antibody is connected to the linker-payload (a) through the cysteine residues of said antibody to either carbon of the 5 membered succinimide ring of compound II or (b) through the cysteine residues of said antibody to either carbon of the ring opened compound III comprising the structures:

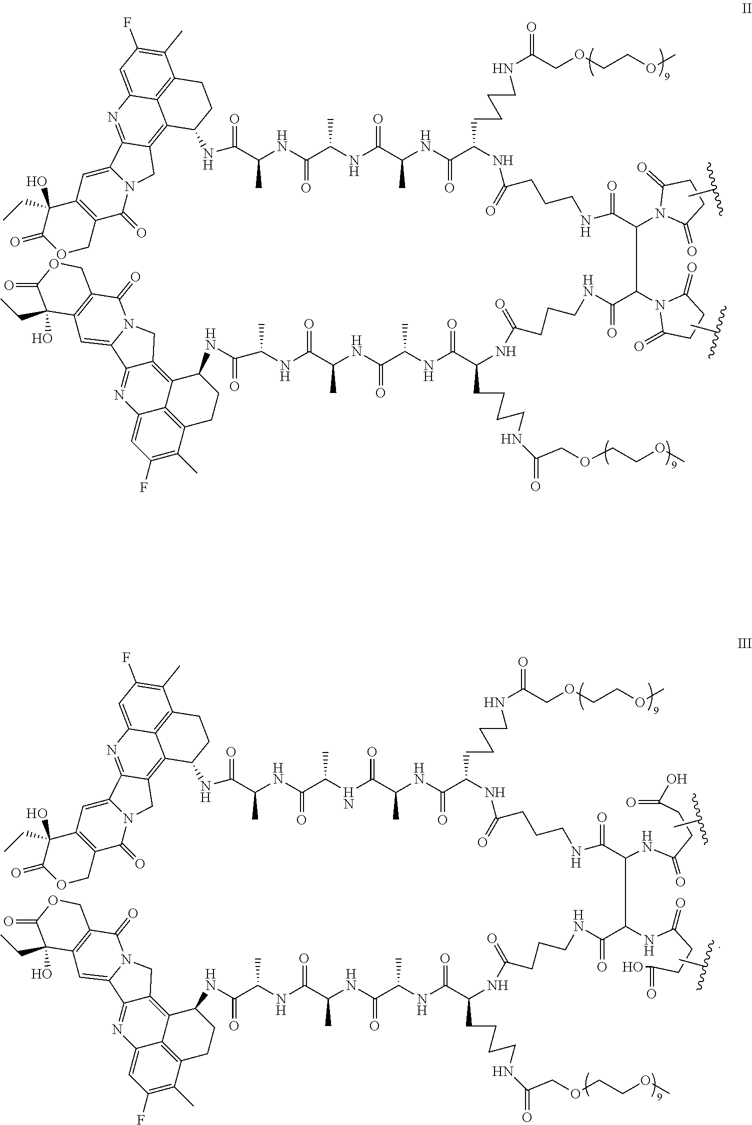

Embodiment 5. The bispecific anti-EGFR/c-Met ADC of embodiment 4, wherein the bispecific anti-EGFR/c-Met antibody is connected to the linker-payload through the cysteine residues of said antibody to either carbon of the 5 membered succinimide ring compound II that comprises the structure:

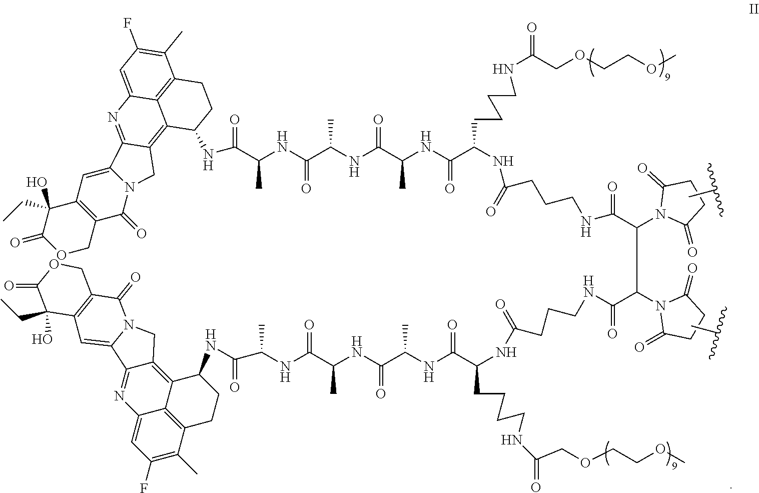

Embodiment 6. The bispecific anti-EGFR/c-Met ADC of embodiment 4, wherein the bispecific anti-EGFR/c-Met antibody is connected to the linker-payload through the cysteine residues of said antibody to either carbon of the 5 membered succinimide ring compound III that comprises the structure:

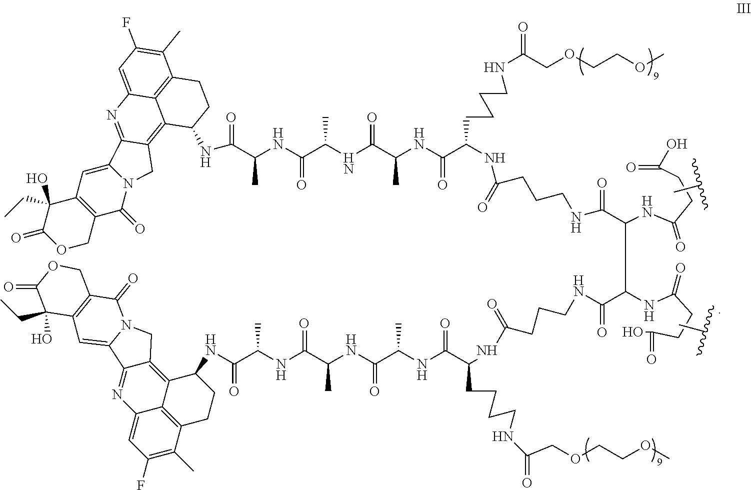

Embodiment 7. The bispecific anti-EGFR/c-Met ADC of any one of the embodiments 1-5, wherein the bispecific anti-EGFR/c-Met ADC comprises Formula I:

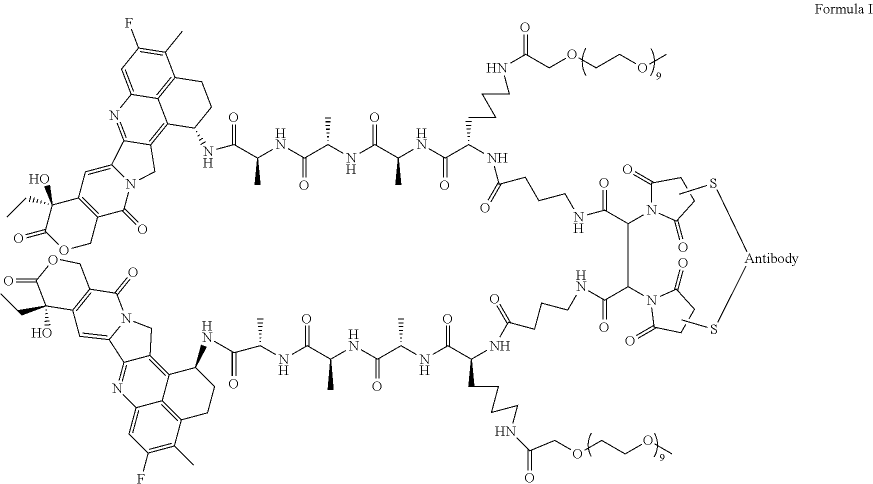

Embodiment 8. The bispecific anti-EGFR/c-Met ADC of any one of the embodiments 1~4 and 6, wherein the bispecific anti-EGFR/c-Met ADC comprises Formula II:

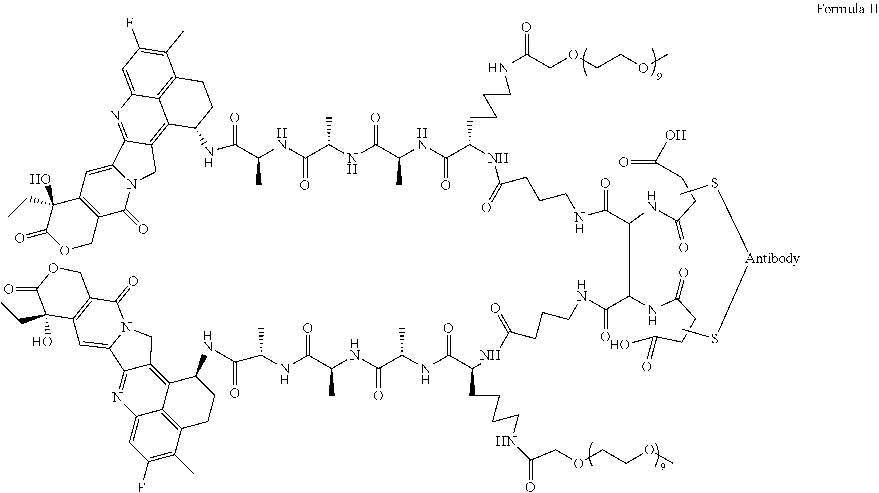

Embodiment 9. The bispecific anti-EGFR/c-Met ADC of any one of the previous embodiments, wherein:
the HC1 comprises a variable region (VH1) comprising the amino acid sequence of SEQ ID NO: 13;
the LC1 comprises a variable region (VL1) comprising the amino acid sequence of SEQ ID NO: 14;
the HC2 comprises a variable region (VH2) comprising the amino acid sequence of SEQ ID NO: 15; and
the LC2 comprises a variable region (VL2) comprising the amino acid sequence of SEQ ID NO: 16.

Embodiment 10. The bispecific anti-EGFR/c-Met ADC of any one of the previous embodiments, wherein the bispecific anti-EGFR/c-Met antibody is an IgG1 isotype.

Embodiment 11. The bispecific anti-EGFR/c-Met ADC of any one of the previous embodiments, wherein:
the HC1 comprises the amino acid sequence of SEQ ID NO: 17;
the LC1 comprises the amino acid sequence of SEQ ID NO: 18;
the HC2 comprises the amino acid sequence of SEQ ID NO: 19; and
the LC2 comprises the amino acid sequence of SEQ ID NO: 20.

Embodiment 12. The bispecific anti-EGFR/c-Met ADC of any one of the previous embodiments, wherein the bispecific anti-EGFR/c-Met antibody comprises a biantennary glycan structure with a fucose content between 0% to 20%.

Embodiment 13. The bispecific anti-EGFR/c-Met ADC of any one of the previous embodiments, wherein the bispecific anti-EGFR/c-Met antibody is amivantamab.

Embodiment 14. The bispecific anti-EGFR/c-Met ADC of any one of the previous embodiments, wherein the bispecific anti-EGFR/c-Met antibody drug conjugate comprises Formula I-A:

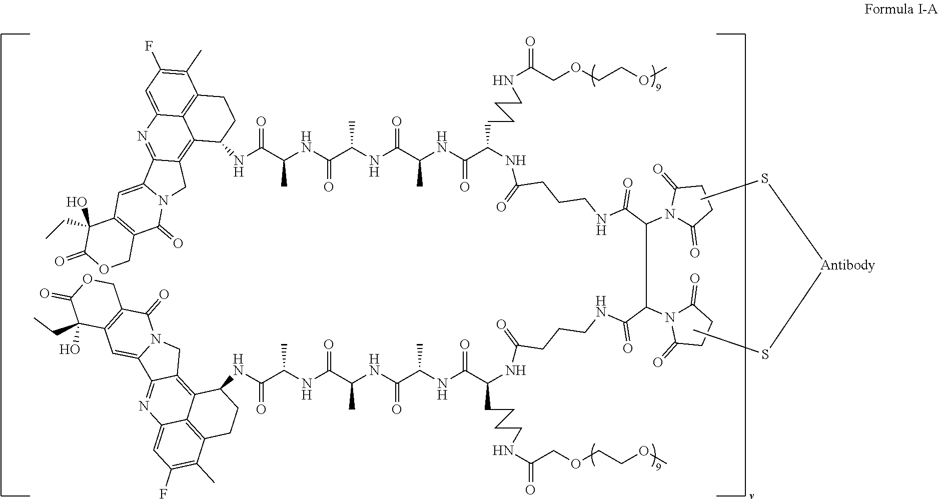

wherein v is 1, 2, 3 or 4.

Embodiment 15. The bispecific anti-EGFR/c-Met ADC of any one of the previous embodiments, wherein the bispecific anti-EGFR/c-Met antibody drug conjugate comprises Formula II-A:

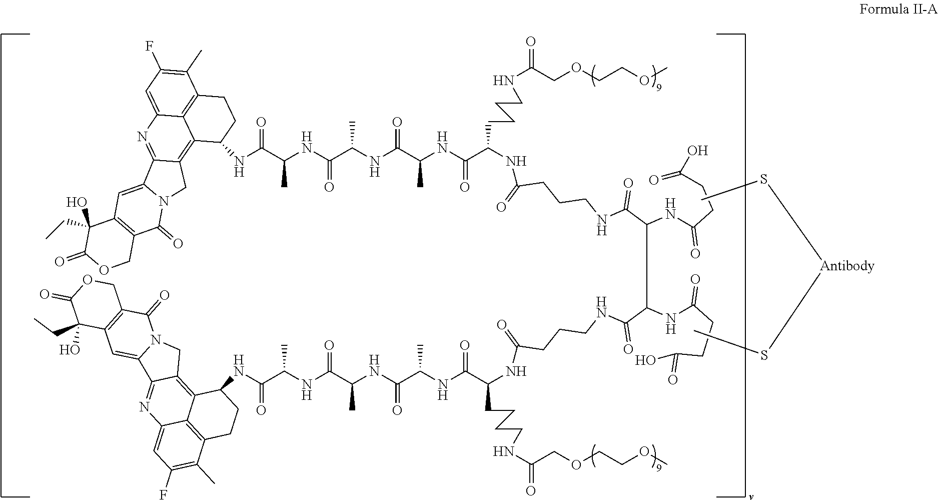

wherein v is 1, 2, 3 or 4.

Embodiment 16. The bispecific anti-EGFR/c-Met ADC of any one of the previous embodiments, comprising a drug-to-antibody ratio (DAR) of about 2 to about 8.

Embodiment 17. The bispecific anti-EGFR/c-Met ADC of any one of the previous embodiments, having a drug-to-antibody ratio (DAR) of about 4.

Embodiment 18. The bispecific anti-EGFR/c-Met ADC of embodiment 14, wherein the bispecific anti-EGFR/c-Met antibody drug conjugate comprises Formula I-A:

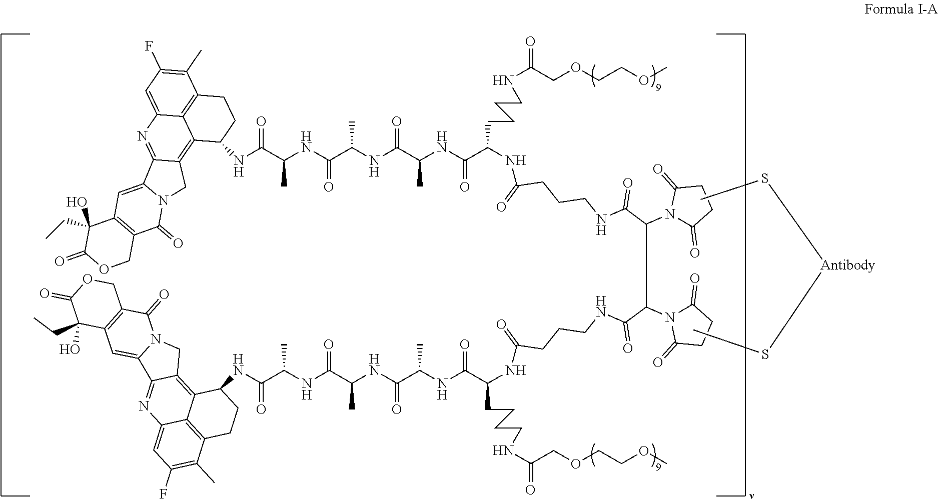

wherein v is 2.

Embodiment 19. The bispecific anti-EGFR/c-Met ADC of claim 15, wherein the bispecific anti-EGFR/c-Met antibody drug conjugate comprises Formula II-A:

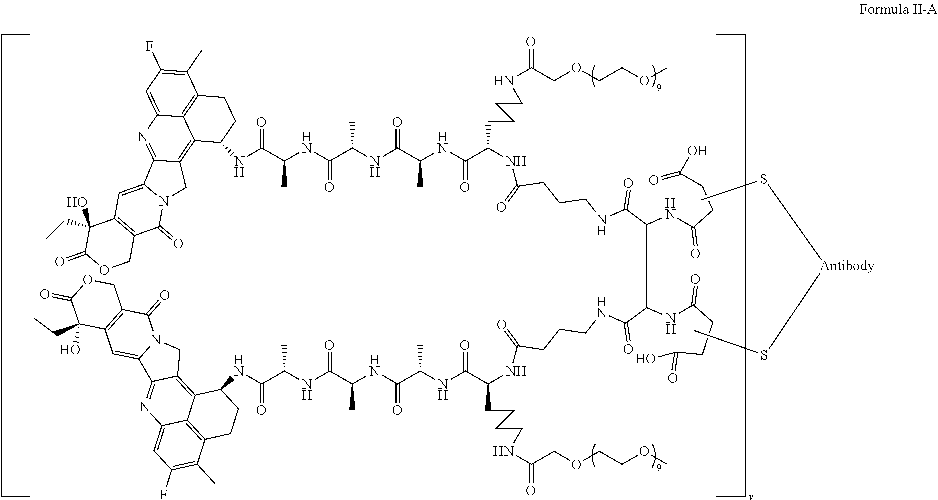

wherein v is 2.

Embodiment 20. The bispecific anti-EGFR/c-Met antibody of any one of the previous embodiments, may be connected or conjugated to the dual maleimide group through one carbon atom in each maleimide. In some embodiments, each maleimide may attach to the bispecific anti-EGFR/c-Met antibody at either carbon of the relevant CH₂ group of the 5-membered ring. In other embodiments, said connection or conjugation is at one or more native or introduced cysteine residues of said antibody. In other embodiments, said connection or conjugation is at one or more interchain cysteine residues of said antibody. In other embodiments, said connection or conjugation is at one or more interchain cysteine residues making up the HC-LC disulfide bond of said antibody. In other embodiments, said connection or conjugation is limited at cysteine residues making up the hinge disulfide bonds of said antibody. In other embodiments, said connection or conjugation is not at cysteine residues making up the hinge disulfide bonds of said antibody. In other embodiments, said connection or conjugation is at the interchain cysteine residues making up the HC-LC disulfide bonds of said antibody wherein said connection or conjugation results in a DAR of 2 to 8. In other embodiments, said connection or conjugation is at the interchain cysteine residues making up the HC-LC disulfide bonds of said antibody wherein said connection or conjugation results in a DAR of about 4, 6 or 8. In other embodiments, said connection or conjugation is at the interchain cysteine residues making up the HC-LC disulfide bonds of said antibody wherein said connection or conjugation results in a DAR of about 4.

Embodiment 21. A bispecific anti-EGFR/c-Met antibody drug conjugate (ADC) comprising a bispecific anti-EGFR/c-Met antibody that comprises:
a first heavy chain (HC1) comprising a heavy chain complementarity determining region 1 (HCDR1) comprising the amino acid sequence of SEQ ID NO: 1, a HCDR2 comprising the amino acid sequence of SEQ ID NO: 2, and a HCDR3 comprising the amino acid sequence of SEQ ID NO: 3; a first light chain (LC1) comprising a light chain complementarity determining region 1 (LCDR1) comprising the amino acid sequence of SEQ ID NO: 4, a LCDR2 comprising the amino acid sequence of SEQ ID NO: 5, and a LCDR3 comprising the amino acid sequence of SEQ ID NO: 6;
a second heavy chain (HC2) comprising a HCDR1 comprising the amino acid sequence of SEQ ID NO: 7, a HCDR2 comprising the amino acid sequence of SEQ ID NO: 8, and a HCDR3 comprising the amino acid sequence of SEQ ID NO: 9; and a second light chain (LC2) comprising a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12; and
wherein the bispecific anti-EGFR/c-Met antibody drug conjugate (ADC) comprises Formula I-A:

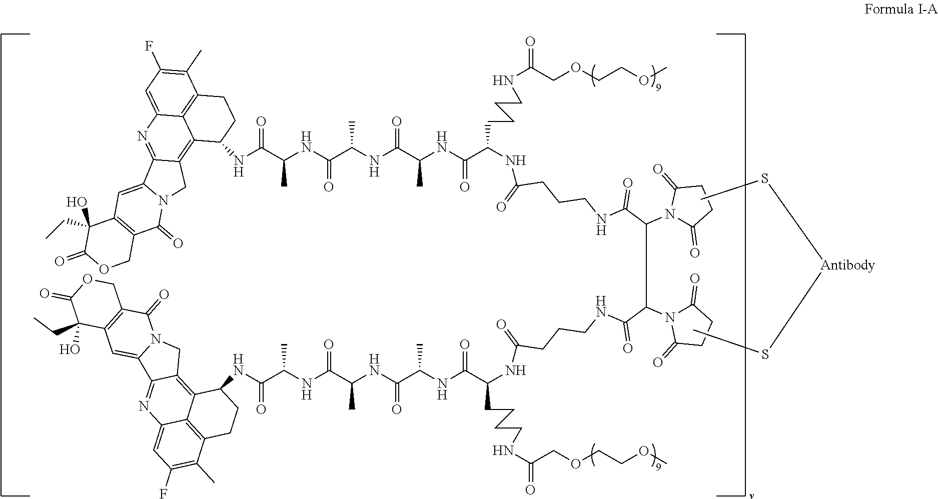

wherein the antibody is the anti-EGFR/c-Met bispecific antibody, v is 2; and
wherein the bispecific anti-EGFR/c-Met antibody drug conjugate (ADC) comprises conjugation at the interchain cysteine residues making up the HC-LC disulfide bonds resulting in a DAR of 4.

Embodiment 22. A bispecific anti-EGFR/c-Met antibody drug conjugate (ADC) comprising a bispecific anti-EGFR/c-Met antibody that comprises:
a HC1 comprising a variable region (VH1) comprising the amino acid sequence of SEQ ID NO: 13;
a LC1 comprising a variable region (VL1) comprising the amino acid sequence of SEQ ID NO: 14;
a HC2 comprising a variable region (VH2) comprising the amino acid sequence of SEQ ID NO: 15; and
a LC2 comprising a variable region (VL2) comprising the amino acid sequence of SEQ ID NO: 16; and
wherein the bispecific anti-EGFR/c-Met antibody drug conjugate (ADC) comprises Formula I-A:

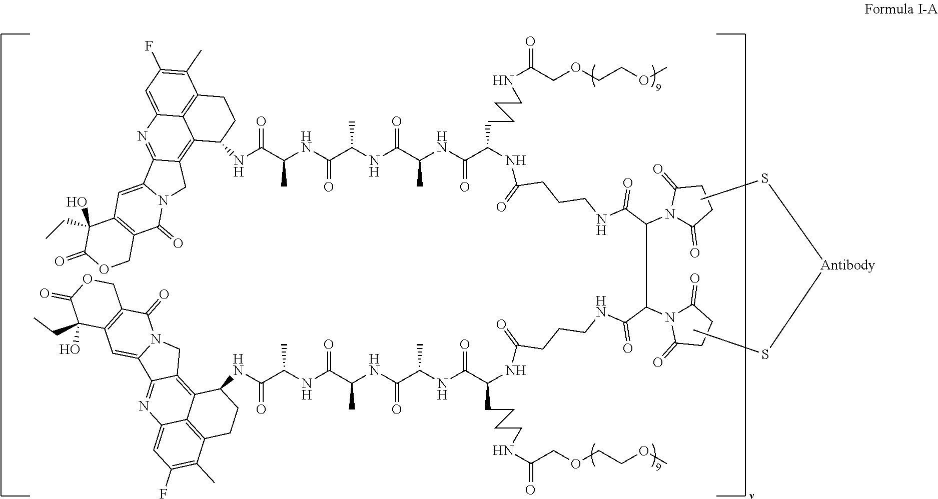

wherein the antibody is the anti-EGFR/c-Met bispecific antibody, v is 2; and
wherein the bispecific anti-EGFR/c-Met antibody drug conjugate (ADC) comprises conjugation at the interchain cysteine residues making up the HC-LC disulfide bonds resulting in a DAR of 4.

Embodiment 23. A bispecific anti-EGFR/c-Met antibody drug conjugate (ADC) comprising a bispecific anti-EGFR/c-Met antibody that comprises:
a HC1 comprising the amino acid sequence of SEQ ID NO: 17;
a LC1 comprising the amino acid sequence of SEQ ID NO: 18;
a HC2 comprising the amino acid sequence of SEQ ID NO: 19; and
a LC2 comprising the amino acid sequence of SEQ ID NO: 20; and
wherein the bispecific anti-EGFR/c-Met antibody drug conjugate (ADC) comprises Formula I-A:

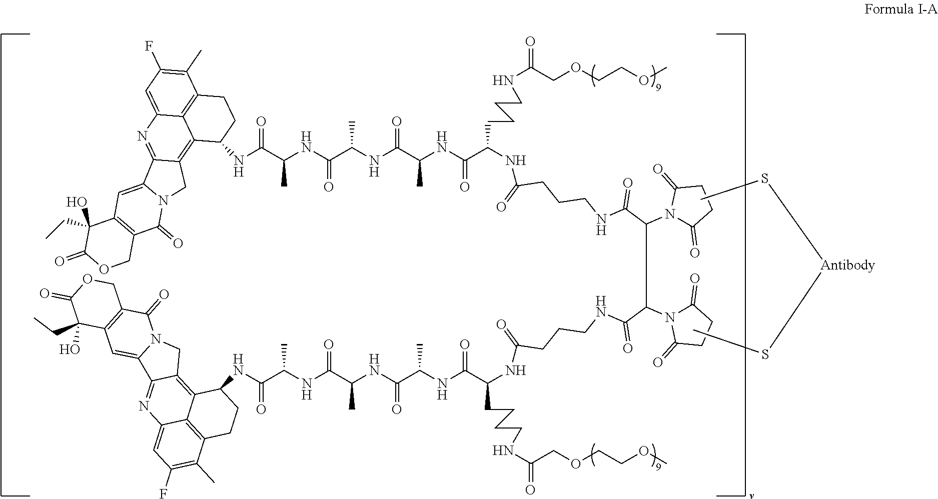

wherein the antibody is the anti-EGFR/c-Met bispecific antibody, v is 2; and
wherein the bispecific anti-EGFR/c-Met antibody drug conjugate (ADC) comprises conjugation at the interchain cysteine residues making up the HC-LC disulfide bonds resulting in a DAR of 4.

Embodiment 24. A bispecific anti-EGFR/c-Met antibody drug conjugate (ADC) comprising a bispecific anti-EGFR/c-Met antibody that comprises:
a first heavy chain (HC1) comprising a heavy chain complementarity determining region 1 (HCDR1) comprising the amino acid sequence of SEQ ID NO: 1, a HCDR2 comprising the amino acid sequence of SEQ ID NO: 2, and a HCDR3 comprising the amino acid sequence of SEQ ID NO: 3; a first light chain (LC1) comprising a light chain complementarity determining region 1 (LCDR1) comprising the amino acid sequence of SEQ ID NO: 4, a LCDR2 comprising the amino acid sequence of SEQ ID NO: 5, and a LCDR3 comprising the amino acid sequence of SEQ ID NO: 6;
a second heavy chain (HC2) comprising a HCDR1 comprising the amino acid sequence of SEQ ID NO: 7, a HCDR2 comprising the amino acid sequence of SEQ ID NO: 8, and a HCDR3 comprising the amino acid sequence of SEQ ID NO: 9; and a second light chain (LC2) comprising a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12; and
wherein the bispecific anti-EGFR/c-Met antibody drug conjugate (ADC) comprises Formula IIA:

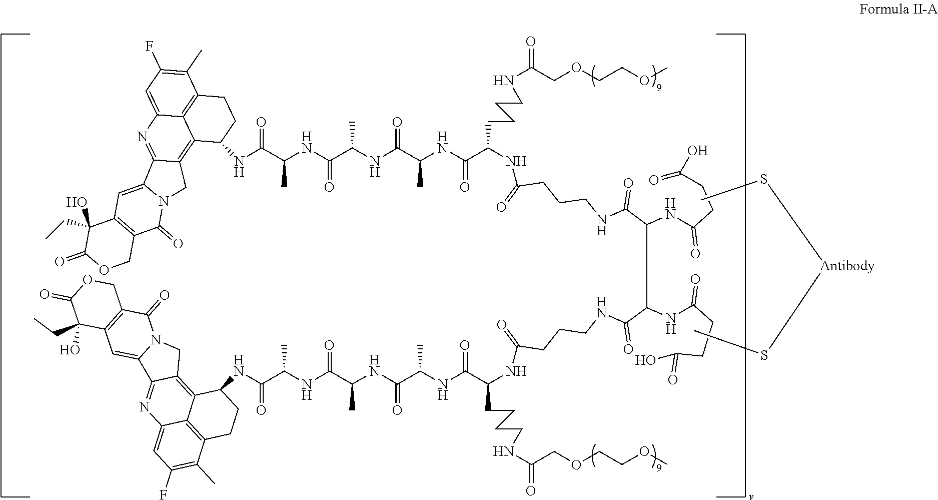

wherein the antibody is anti-EGFR/c-Met bispecific antibody, v is 2; and
wherein the bispecific anti-EGFR/c-Met antibody drug conjugate (ADC) comprises conjugation at the interchain cysteine residues making up the HC-LC disulfide bonds resulting in a DAR of 4.

Embodiment 25. A bispecific anti-EGFR/c-Met antibody drug conjugate (ADC) comprising a bispecific anti-EGFR/c-Met antibody that comprises:
a HC1 comprising a variable region (VH1) comprising the amino acid sequence of SEQ ID NO: 13;
a LC1 comprising a variable region (VL1) comprising the amino acid sequence of SEQ ID NO: 14;
a HC2 comprising a variable region (VH2) comprising the amino acid sequence of SEQ ID NO: 15; and
a LC2 comprising a variable region (VL2) comprising the amino acid sequence of SEQ ID NO: 16; and
wherein the bispecific anti-EGFR/c-Met antibody drug conjugate (ADC) comprises Formula IIA:

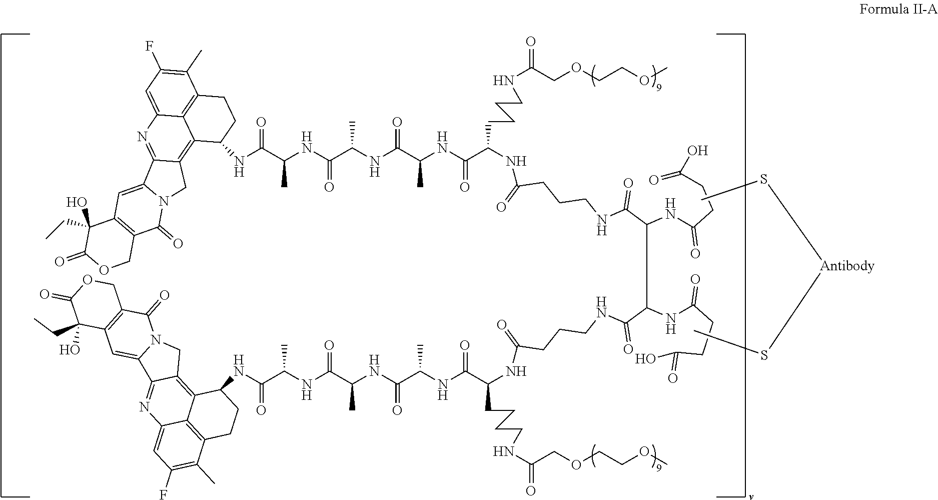

wherein the antibody is anti-EGFR/c-Met bispecific antibody, v is 2; and
wherein the bispecific anti-EGFR/c-Met antibody drug conjugate (ADC) comprises conjugation at the interchain cysteine residues making up the HC-LC disulfide bonds resulting in a DAR of 4.

Embodiment 26. A bispecific anti-EGFR/c-Met antibody drug conjugate (ADC) comprising a bispecific anti-EGFR/c-Met antibody that comprises:
a HC1 comprising the amino acid sequence of SEQ ID NO: 17;
a LC1 comprising the amino acid sequence of SEQ ID NO: 18;
a HC2 comprising the amino acid sequence of SEQ ID NO: 19; and
a LC2 comprising the amino acid sequence of SEQ ID NO: 20;
and wherein the bispecific anti-EGFR/c-Met antibody drug conjugate (ADC) comprises Formula IIA:

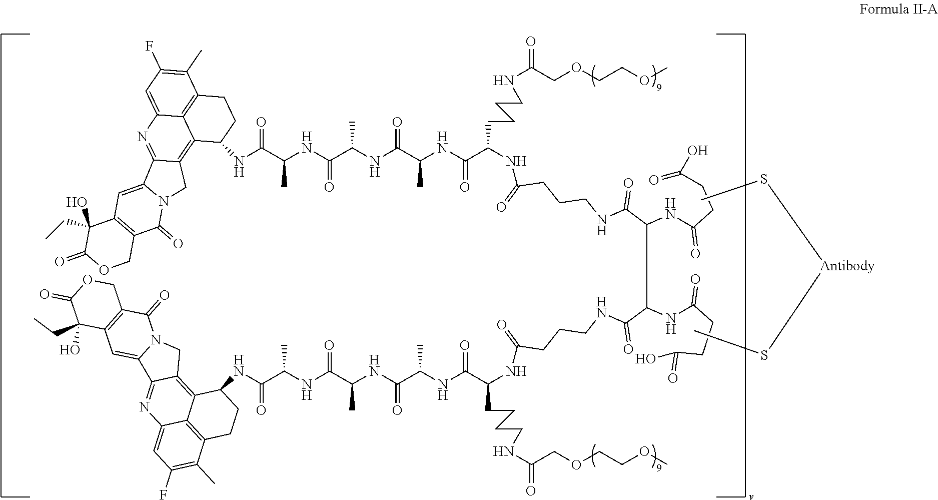

wherein the antibody is anti-EGFR/c-Met bispecific antibody, v is 2; and
wherein the bispecific anti-EGFR/c-Met antibody drug conjugate (ADC) comprises conjugation at the interchain cysteine residues making up the HC-LC disulfide bonds resulting in a DAR of 4.

Embodiment 27. A pharmaceutical composition comprising the bispecific anti-EGFR/c-Met ADC of any one of the previous embodiments and a pharmaceutically acceptable excipient.

Embodiment 28. A method of treating an EGFR expressing cancer and/or c-Met expressing cancer in a patient in need thereof, the method comprising administering the bispecific anti-EGFR/c-Met ADC of any one of embodiments 1-26 or the pharmaceutical composition of embodiment 27 to the patient to thereby treat the cancer.

Embodiment 29. The method of embodiment 28, wherein the cancer is lung cancer, colorectal cancer, or head and neck cancer.

Embodiment 30. The method of embodiment 29, wherein the lung cancer is epithelial lung cancer.

Embodiment 30. The method of embodiment 29, wherein the lung cancer is non-small cell lung cancer (NSCLC).

Embodiment 31. The method of embodiment 30, wherein the NSCLC is squamous cell carcinoma, large cell carcinoma, or adenocarcinoma.

Embodiment 32. A method for making a hydrolyzed succinimide thioether ring of the bispecific anti-EGFR/c-Met antibody drug conjugate (ADC) of any one of embodiments 1-31 or the pharmaceutical composition of embodiment 27, the method of hydrolysis of the succinimide-thioether ring results in ring opening to the hydrolyzed structure. The method comprises that succinimide thioether ring of the ADC solution subjected to buffer exchange, under adjusted to pH 7.1 by the addition of 400 mM Na₂HPO₄ and then stirred at 30° C. for 42-48 hr. The resulting solution was diafiltered into formulation buffer, filter sterilized, and characterized to determine DAR, concentration, % monomer, etc. using standard analytical techniques. The method is as depicted below:

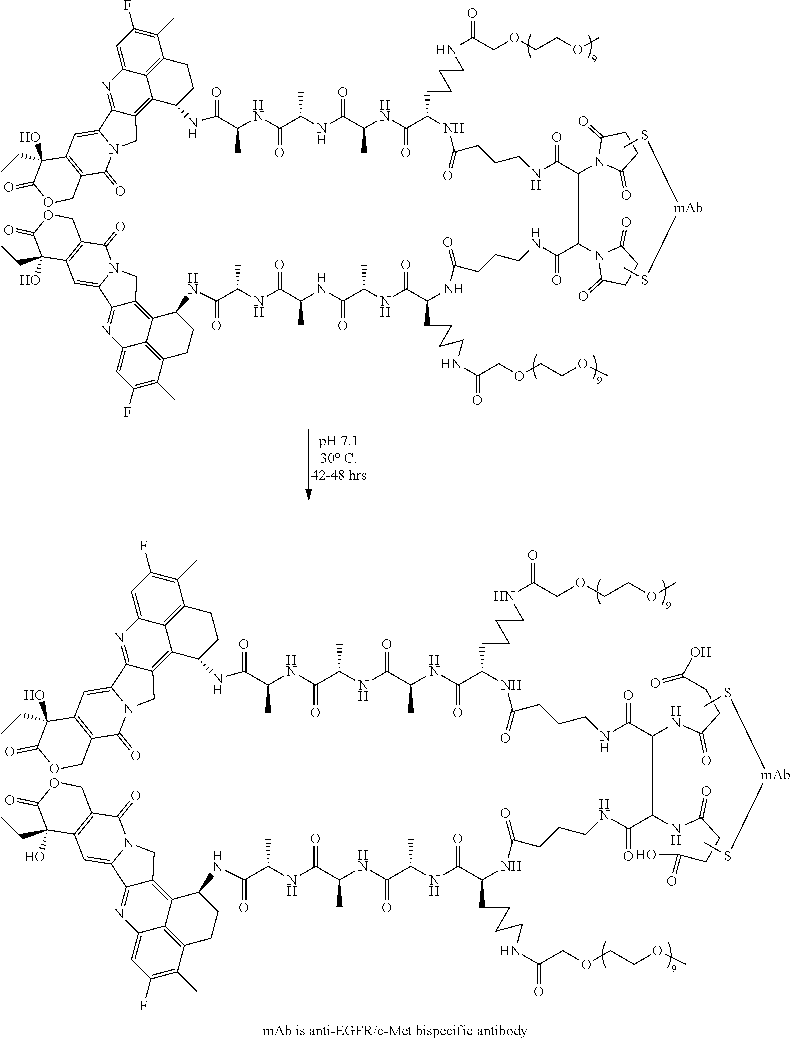

### EXAMPLES

The following examples are provided to further describe some of the embodiments disclosed herein. The examples are intended to illustrate, not to limit, the disclosed embodiments.

Ab1-ADC is identified as having the bispecific anti-EGFR/c-Met antibody conjugated to linker-payload having the bismaleimide linker moiety comprising the structures below:

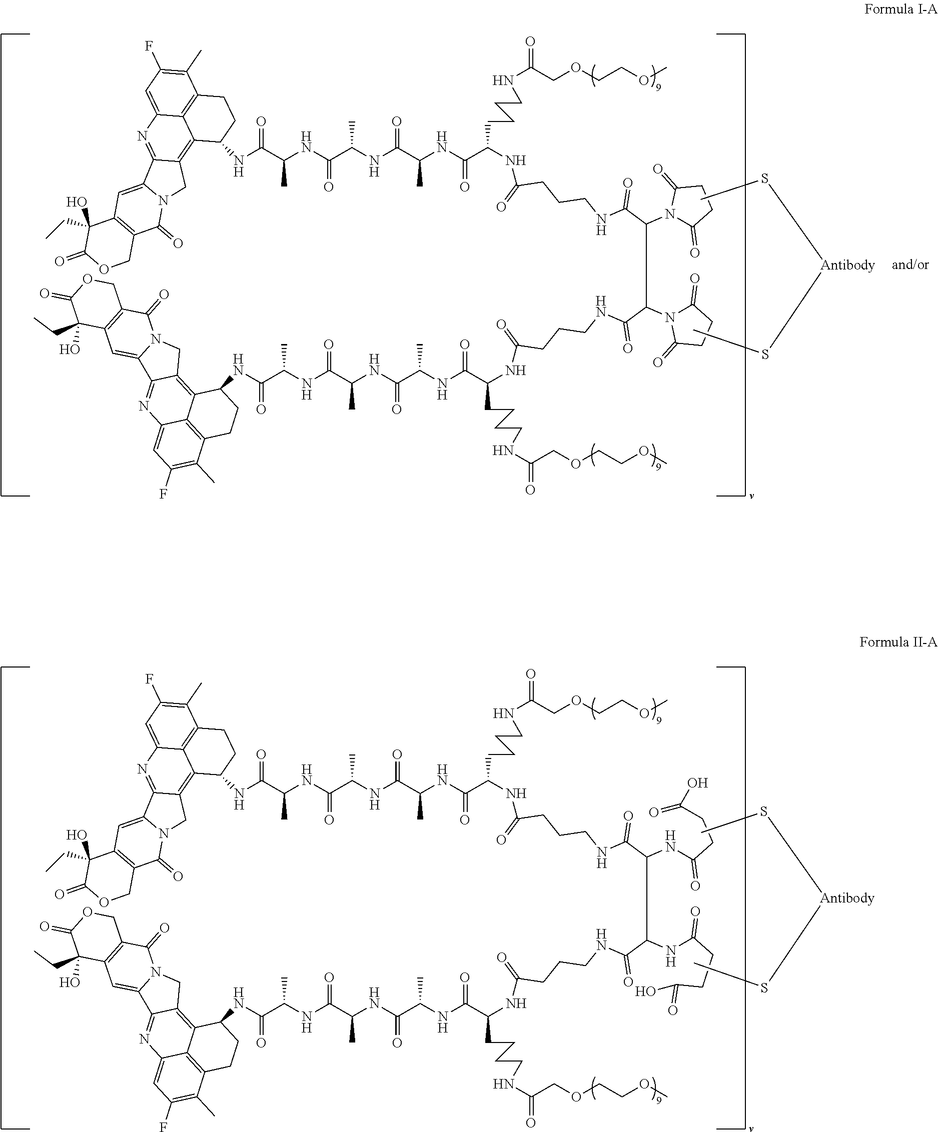

Ab1-ADC2 is identified as having the bispecific anti-EGFR/c-Met antibody conjugated to linker-payload having the mono-maleimide linker moiety.

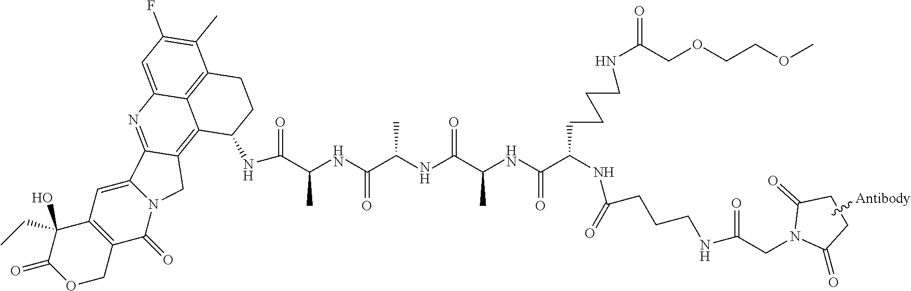

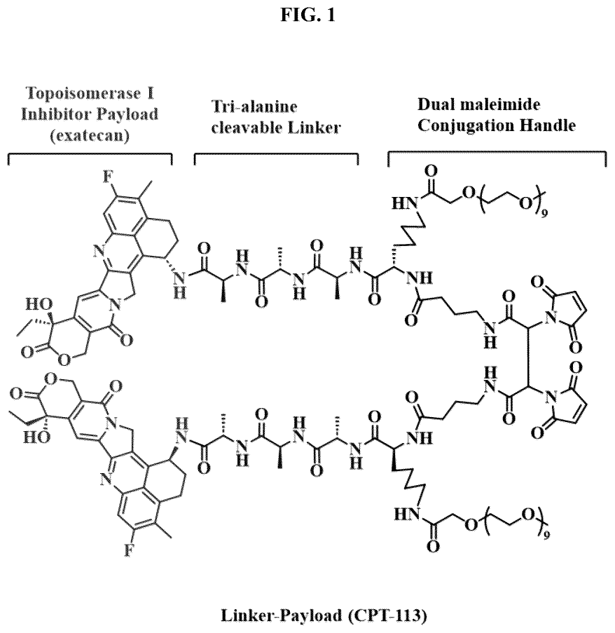

Ab1-ADC is an exemplary bispecific anti-EGFR/c-Met ADC that comprises amivantamab conjugated to the payload linker depicted in FIG. 1. Ab1-ADC comprises a structure suitable for developability, including tolerability and other characteristics described herein.

### Example 1. EGFR and MET Expression on Exemplary Cancer Cell Lines

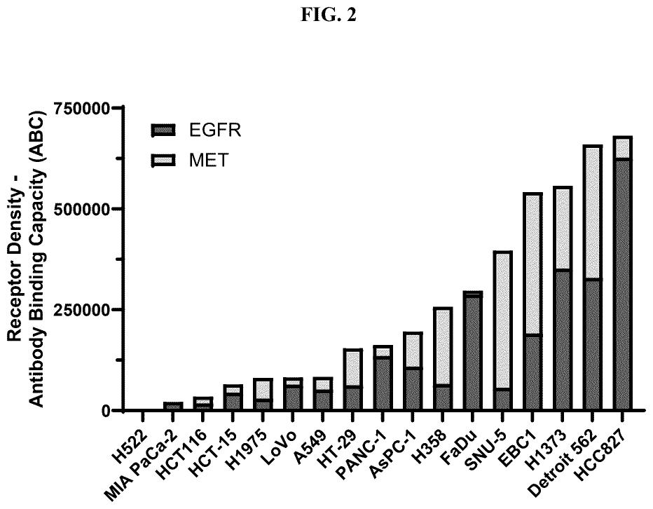

EGFR and MET surface expression was characterized on a panel of lung (HCC827, H1373, EBC-1, H358, A549, H1975, H522), CRC (HT-29, LoVo, HCT-15, HCT116), HNSCC (Detroit562, FaDu), gastric (SNU-5), and pancreatic cancer (AsPC-1, PANC-1, MIA PaCa-2) cell lines by flow cytometry using the monovalent single-arm EGFR and MET binders from Ab1-ADC. EGFR was highly expressed (antibody binding capacity [ABC]>300,000) in the lung cancer lines HCC827 and H1373 and the HNSCC cell line Detroit562 (FIG. 2). Similarly, MET was highly expressed (ABC >300,000) in Detroit562 cells as well as in the NSCLC line EBC-1 and gastric line SNU-5. Of note, in HCC827 and FaDu cells, EGFR expression was much higher than MET, whereas in EBC-1, SNU-5, and H358 cells, MET expression was much higher than EGFR. H522 cells were negative for both EGFR and MET expression.

EGFR and MET expression levels on a panel of cancer cell lines is shown on FIG. 2. Flow-cytometry-based determination of membrane expression of EGFR and MET using the monovalent, phycoerythrin-conjugated MET and EGFR binders is shown.

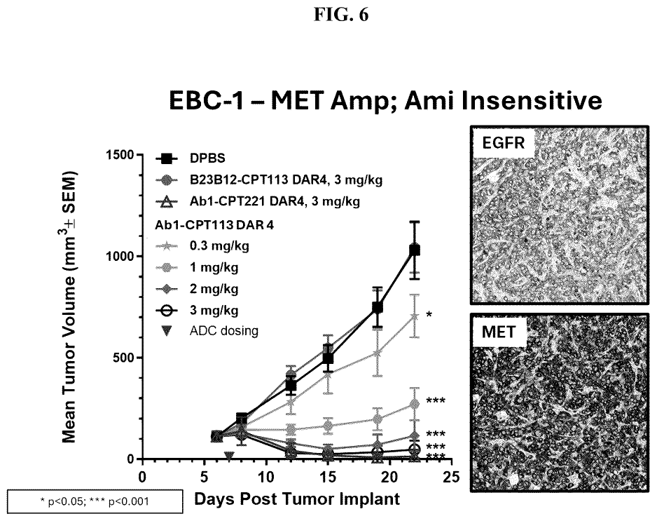

Four cell lines, with varying EGFR and MET levels when evaluated by antibody binding capacity, were established as in vivo cell-derived xenograft (CDX) models for efficacy studies with Ab1-ADC: H1975 (EGFR low, MET low), EBC-1 (EGFR medium, MET high), H1373 (EGFR high, MET medium), and A549 (EGFR low, MET low). When IHC staining was performed for EGFR and MET using commercially available antibodies (ie, EGFR: Clone D38B1; MET: Clone SP44) on ex vivo tumors from these CDX models (FIG. 6-FIG. 9), tumor staining intensities were mostly consistent with the in vitro data showing EBC-1 and H1373 had high MET and EGFR expression, respectively, whereas H1975 had moderate staining and A549 displayed low-intensity staining for both EGFR and MET.

### Example 2. In Vitro Binding of Ab1-ADC to Tumor Cells

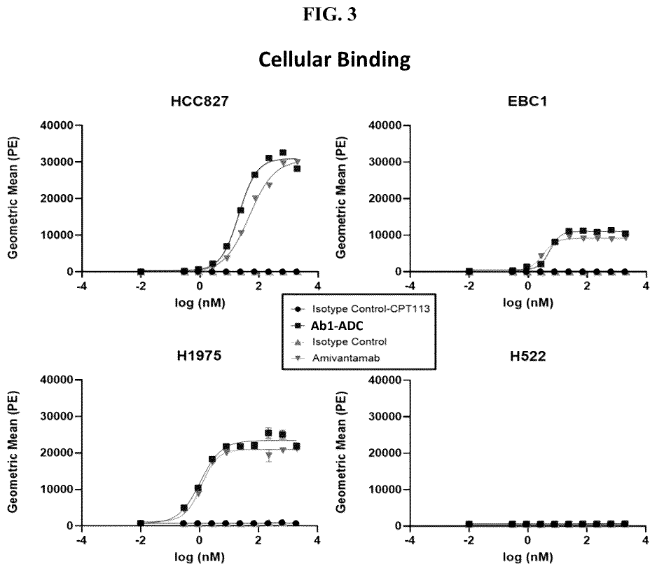

The in vitro cell binding of Ab1-ADC was compared to the binding of amivantamab and non-targeting isotype control antibodies using several EGFR- and MET-expressing cells lines and the EGFR- and MET-negative cell line H522. To minimize antibody internalization throughout the assay, all incubations were performed at 4° C. for 30-45 minutes. Ab1-ADC binding was specific to EGFR- and MET-expressing cells and affinity was consistent with amivantamab indicating that the conjugation of the cytotoxic payload did not alter the binding of the parent BsAb to tumor cells (FIG. 3). The 50% effective concentration (EC50) values for Ab1-ADC binding ranged from approximately 1 to 21 nM and no binding was detected to the target-negative cell line H522, or by the isotype control antibodies (Table 2).

Binding of Ab1-ADC to tumor cell lines assessed by flow cytometry is shown on FIG. 3.

TABLE 2
Cellular Binding of Exemplary Bispecific
Anti-EGFR/c-Met Antibody-Drug Conjugate

| NSCLC Cell | | Ab1-ADC | Amivantamab |
|---|---|---|---|
| Lines | EGFR/MET status | EC₅₀ (nM) | EC₅₀ (nM) |
| HCC827 (a) | EGFR Amp/Del19 | 20.74 | 46.38 |
| EBC-1 (sq) | MET Amp | 5.372 | 3.003 |
| H1975 (a) | EGFR L858R/T790M | 1.057 | 1.149 |
| H522 (a) | Lung Adeno | ND | ND |

(a)—adenocarcinoma;
(sq)—squamous

### Example 3. Internalization Studies

### Internalization of Amivantamab

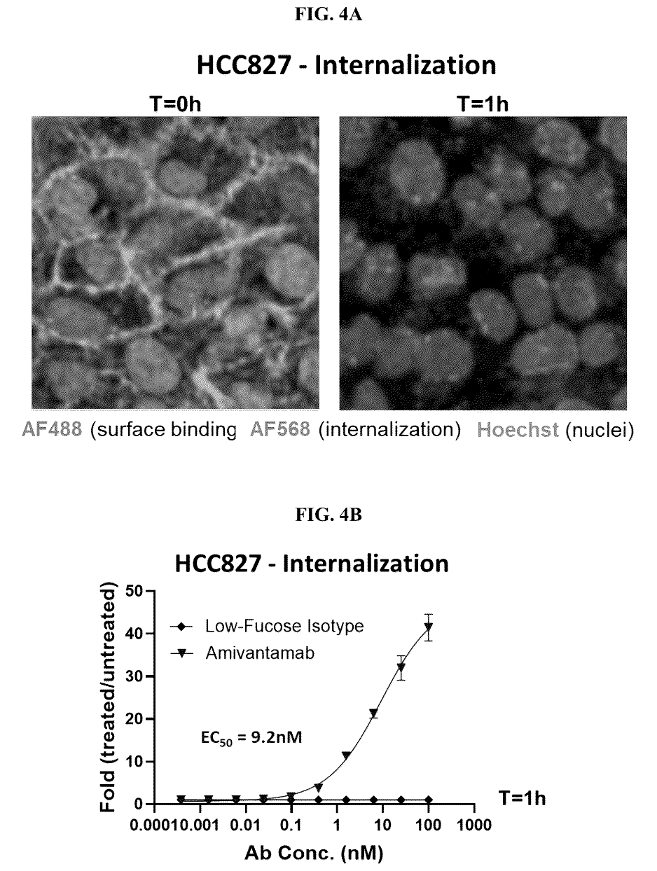

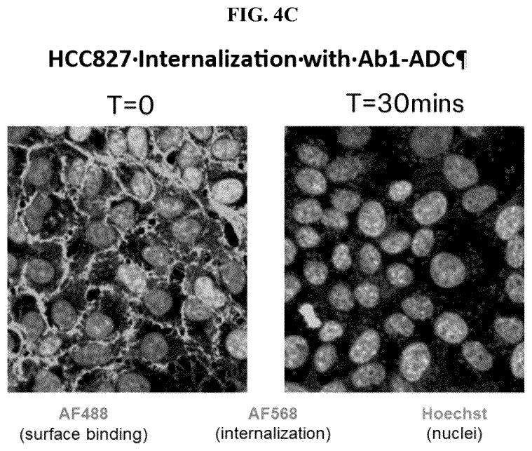

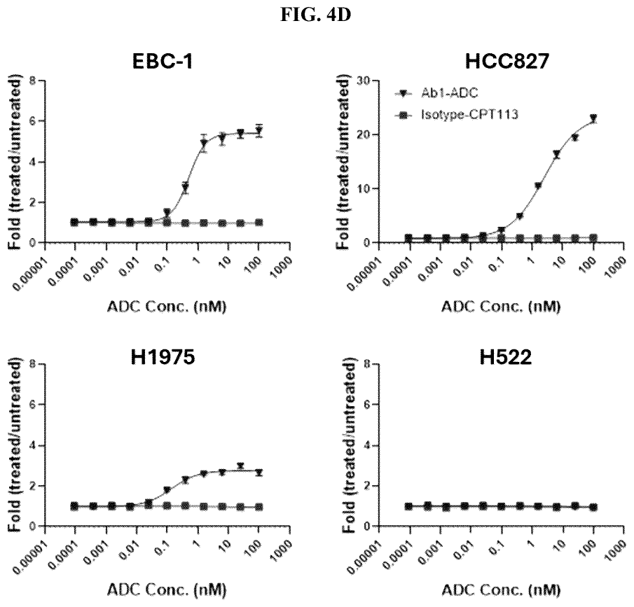

Using high-content confocal imaging, the internalization of amivantamab, was evaluated in a panel of cell lines with diverse EGFR and MET expression levels. Briefly, using secondary anti-IgG antibodies conjugated to the fluorophores AF488 and AF568 on intact and permeabilized cells, respectively, surface-bound and internalized amivantamab was quantified by fluorescent emission at the fluorophore detection wavelengths. Amivantamab demonstrated robust internalization in all cell lines tested with a maximal increase of 41.7× of AF568 emission compared to isotype control antibody in HCC827 cells (FIG. 4A-FIG. 4B). The EC50 values for internalization ranged from 0.3 to 9.2 nM across the panel of cell lines and the fold increase over isotype control corresponded with the receptor densities of EGFR and MET.

### Internalization of Ab1-ADC

High-content imaging assays were conducted to assess the internalization of Ab1-ADC and the parental BsAb amivantamab. Cells were plated and allowed to incubate overnight. The following day the antibodies were added to the cells, and plates returned to the incubator. Next, 10% paraformaldehyde was added to the wells, followed by 10 minutes incubation, wash with PBS, and block using 5% goat serum. Following the PBS wash, staining of surface-bound antibodies was performed by incubating with secondary anti-human IgG-Alexa488. After washing cells, surface blocking using unlabeled anti-human IgG secondary antibody and PBS wash, cells were fixed a second time with 4% paraformaldehyde, incubate for 10 minutes at room temperature. After washing the plate, cells were permeabilize by adding permeabilization solution (0.1-0.2% TritonX-100 in 5% goat serum) and incubating at room temperature for 10 minutes. After washing the plate twice, internalized antibody was stained by adding the internal staining solution (Final concentration: Hoechst 1:5000, anti-human IgG-Alexa568 1:1000, Cellmask Deep Red 1:5000 in 5% goat serum) and incubating at room temperature for 1 HR. After washing cells, plates cells were stored in PBS at 4° C. Plates were analyzed on the Opera Phenix or CellVoyager CV8000, images analyzed using Phaedra, and data analysis with GeneData Screener and ShinyApp. Ab1-ADC demonstrated rapid internalization in vitro across the panel of cell lines (FIG. 4C-FIG. 4D).

### Example 4. Ab1-ADC-Induced Cytotoxicity in EGFR- and/or MET-Expressing Cell Lines

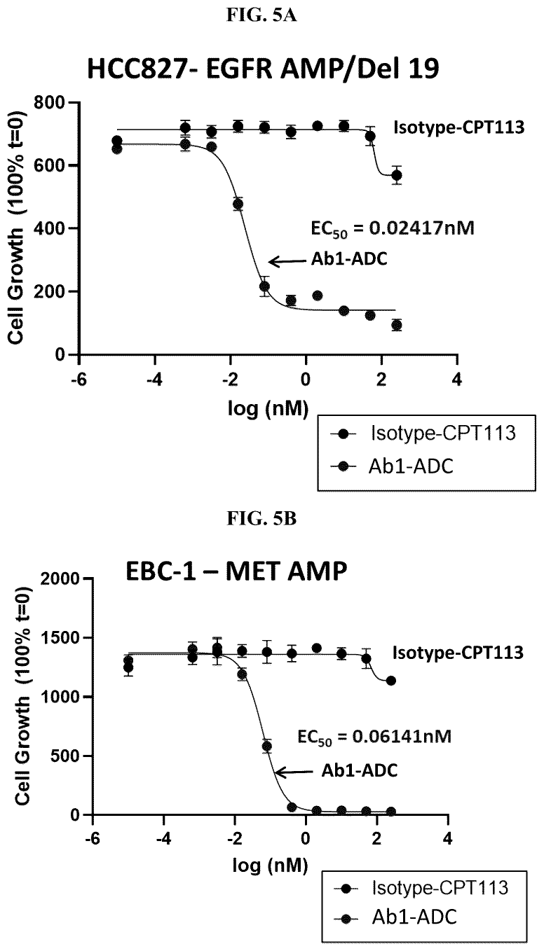

Ab1-ADC-mediated cytotoxicity was evaluated in a panel of cell lines with a diverse range of EGFR and MET expression as well as the antigen-negative cell line H522. With the exception of H1373 cells, Ab1-ADC demonstrated potent cytotoxicity in cell lines with high total expression of EGFR and MET (FIG. 5A-FIG. 5B, Table 3), including the aforementioned models with very high EGFR (HCC827 and FaDu) or MET (EBC-1 and SNU-5) expression levels, indicating both binding arms of the Ab1-ADC were capable of inducing internalization and cytotoxicity. Of the cell lines that demonstrated sensitivity to Ab1-ADC, the MET-amplified gastric cancer cell line SNU-5 was the most sensitive cell line tested in the panel with an EC50 value of 0.55 μM and H358 was the least sensitive at 3.5 nM. Although Ab1-ADC did not demonstrate cytotoxicity in cell lines that did not have high levels of EGFR and/or MET, including H1975 and A549 cells, CPT-116 (III), the major metabolite of CPT-113 (I), was cytotoxic in nearly every cell line with EC50 values ranging from approximately 60 μM to 12 nM. (Table 3). These data indicate Ab1-ADC is a biologically active molecule capable of inducing cytotoxicity in high antigen-expressing cell lines and the major metabolite CPT-116 (III) is capable of inducing cytotoxicity across a large panel of cancer cell lines.

TABLE 3
EC50 values for tumor cell cytotoxicity in a panel of EGFR−
and/or MET− expressing tumor cell lines.

| | Ab1-ADC EC₅₀ (nM) | CPT-116 EC₅₀ (nM) |
|---|---|---|
| Cell line | Day 6 | Day 6 |
| HCC827 | 0.02417 | 0.7348 |
| Detroit562 | 0.2936 | 0.6037 |
| H1373 | ND | 7.788 |
| EBC-1 | 0.06141 | 0.4771 |
| SNU-5 | 0.00055 | 1.105 |
| FaDu | 0.2936 | 0.6320 |
| H358 | 3.500 | 0.4342 |
| AsPC-1 | ND | ND |
| Panc-1 | ND | 3.775 |
| HT-29 | ND | 5.307 |
| A549 | ND | 11.92 |
| Lovo | ND | 6.961 |
| H1975 | ND | 6.461 |
| H522 | ND | 0.06013 |

CPT—camptothecin; EC₅₀—50% effective concentration.
EC₅₀ values were not determined if the dose-response curve did not satisfy one or both of the following criteria: R² has to be >0.9 and the log (95% confidence interval) difference needs to be <1.2.

### Example 5. Efficacy of Ab1-ADC in MET-amplified Xenograft Model EBC-1

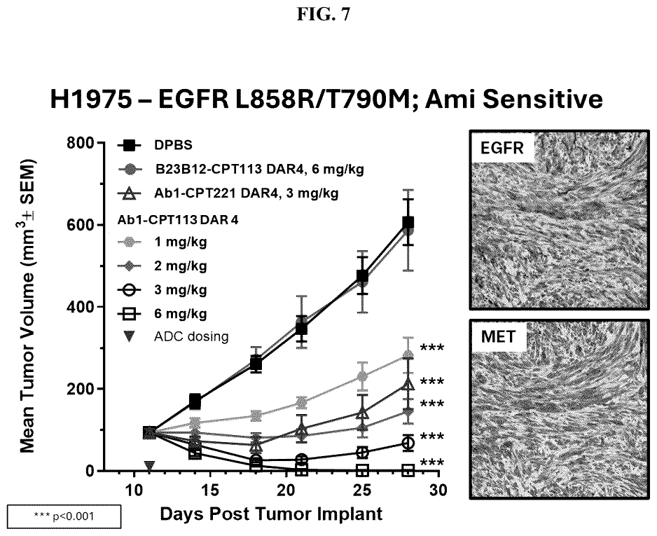

Athymic nude mice bearing established SC EBC-1 xenografts were dosed once IV with either Ab1-ADC at 0.3, 1, 2, or 3 mg/kg, isotype-ADC (B23B12-CPT113, DAR 4) at 3 mg/kg, or Ab1-CPT221 DAR4 at 3 mg/kg. Dulbecco's phosphate buffered saline (DPBS) was used as a vehicle control. Ab1-CPT221 features the same antibody (Ab1) and a different linker-payload, as compared to Ab1-ADC. Ab1-ADC demonstrated unique properties and activity as compared to Ab1-CPT221 DAR4 when administered at similar quantities (see, for example, FIG. 7.) Briefly, tumor cells were implanted on Day 0, followed by treatment on Day 7. Potent antitumor efficacy was observed with Ab1-ADC treatment at 0.3, 1, 2, and 3 mg/kg as assessed by change in mean tumor burden on Day 22 post tumor implantation, with ΔTGI of 36%, 83%, 100%, and 107%, respectively (p<0.001; FIG. 6, Table 4) as compared to isotype-ADC. Ab1-ADC treatment at 1 mg/kg treatment resulted in 3 of 10 partial regressions (PRs) and 1 of 10 complete regression (CR); the 2 mg/kg dose resulted in 2 of 10 PRs and 5 of 10 CRs; the 3 mg/kg dose resulted in % TR of 58% and 1 of 10 PR and 8 of 10 CRs on Day 22. Continued monitoring of the Ab1-ADC treated groups at 2 mg/kg resulted in 2 of 10 PRs and 3 of 10 CRs; the 3 mg/kg dose resulted in 7 of 10 CRs on Day 42. AB1-ADC at 1 mg/kg was identified as the MED in this model with TGI of 74% on Day 22. Amivantamab was not effective in the EBC-1 model (data not shown). Representative IHC images of EBC-1 xenograft tumors stained intensely for both EGFR and MET are shown in FIG. 6.

TABLE 4
Tumor Growth Inhibition in MET and EGFR Dependent NSCLC
Xenografts (EBC-1 - MET AMP; Ami Insensitive) by Exemplary
Bispecific Anti-EGFR/c-Met Antibody-Drug Conjugate

Day 22 vs Isotype-ADC

| Ab1-ADC Dose | % TGI | %ΔTGI | % TR, PR/CR |
|---|---|---|---|
| 0.3 mg/kg | 32 | 36 | — |
| 1 mg/kg | 74 | 83 | 0, 3/1 |
| 2 mg/kg | 90 | 100 | 0, 2/5 |
| 3 mg/kg | 95 | 107 | 58, 1/8 |

### Example 6. Efficacy of Ab1-ADC in EGFR-mutant H1975 CDX Model

Athymic nude mice bearing established SC H1975 xenografts were dosed once IV with either Ab1-ADC at 1, 2, 3, or 6 mg/kg, isotype-ADC (B23B12-CPT113, DAR 4) at 6 mg/kg, or Ab1-CPT221 DAR4 at 3 mg/kg. DPBS was used as a vehicle control. Ab1-CPT221 features the same antibody (Ab1) and a different linker-payload, as compared to Ab1-ADC. Briefly, tumor cells were implanted on Day 0, followed by treatment on Day 11. Significant antitumor efficacy was observed with Ab1-ADC treatment at 1, 2, 3, and 6 mg/kg as assessed by change in mean tumor burden on Day 28 post tumor implantation (p<0.001; FIG. 7, Table 5), with ΔTGI of 62%, 89%, 105%, and 119%, respectively, compared to isotype-ADC-treated control. Ab1-ADC treatment at 2 mg/kg resulted in 2 of 9 PRs; 3 mg/kg resulted in % TR of 27% and 5 of 9 PRs and 1 of 9 CR; 6 mg/kg resulted in % TR of 99% and 2 of 9 PRs and 7 of 9 CRs by Day 28. Continued monitoring of 6 mg/kg dose of Ab1-ADC resulted in % TR of 86% and 2 of 9 PRs and 7 of 9 CRs on Day 49. The lack of biologically significant efficacy observed from treatment with Ab1-ADC at 1 mg/kg (52% TGI on Day 28) demonstrated that 2 mg/kg is the MED in this study. In contrast, amivantamab was effective in the H1975 model at 10 mg/kg dosed twice weekly (q3d-q4d)×3, however, with less tumor inhibition compared to 2 mg/kg of Ab1-ADC. Representative IHC images of H1975 xenograft tumors stained intensely for both EGFR and MET are shown in FIG. 7.

TABLE 5
Tumor Growth Inhibition in MET and EGFR Dependent
NSCLC Xenografts (H1975 - EGFR L858R/T790M;
Ami Sensitive) by Exemplary Bispecific Anti-
EGFR/c-Met Antibody-Drug Conjugate

Day 28 vs Isotype-ADC

| Ab1-ADC Dose | % TGI | %ΔTGI | % TR, PR/CR |
|---|---|---|---|
| 1 mg/kg | 52 | 62 | — |
| 2 mg/kg | 75 | 89 | 0, 2/0 |
| 3 mg/kg | 88 | 105 | 27, 5/1 |
| 6 mg/kg | 100 | 119 | 99, 2/7 |

### Example 7. Efficacy of Ab1-ADC in KRAS-mutant Lung Cancer Xenograft Models

KRAS mutations are counter-indicated for EGFR-targeted therapies, however, as an ADC, Ab1-ADC may overcome these limitations. Therefore, the efficacy of Ab1-ADC was examined in 2 KRAS mutant CDX models that have previously been demonstrated to be resistant to amivantamab (data not shown).

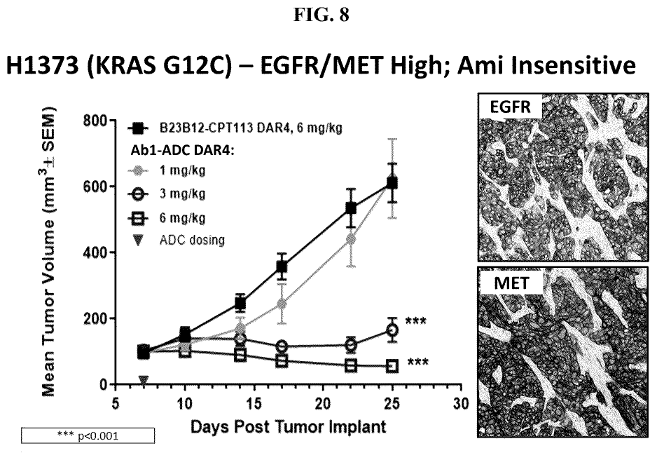

Athymic nude mice bearing established SC H1373, KRAS G12C, xenografts were dosed once IV with Ab1-ADC at 1, 3, and 6 mg/kg or isotype-ADC (B23B12-CPT113, DAR 4) at 6 mg/kg. Significant antitumor efficacy was observed with Ab1-ADC treatment at 3 and 6 mg/kg as assessed by change in mean tumor burden on Day 25 post tumor implantation, with ΔTGI of 88% and 109%, respectively (p<0.001; FIG. 8, Table 6) as compared to isotype-ADC. No TGI was observed with Ab1-ADC at 1 mg/kg and 3 mg/kg (73% TGI) was determined to be the MED in this model. Ab1-ADC treatment at 3 mg/kg resulted in 5 of 10 PRs and 6 mg/kg treatment resulted in % TR of 44% and 9 of 10 PRs by Day 25. Continued monitoring resulted in 5 of 10 PRs at 6 mg/kg dose of Ab1-ADC on Day 42. Representative IHC images of H1373 xenograft tumors stained intensely for both EGFR and MET are shown in FIG. 8.

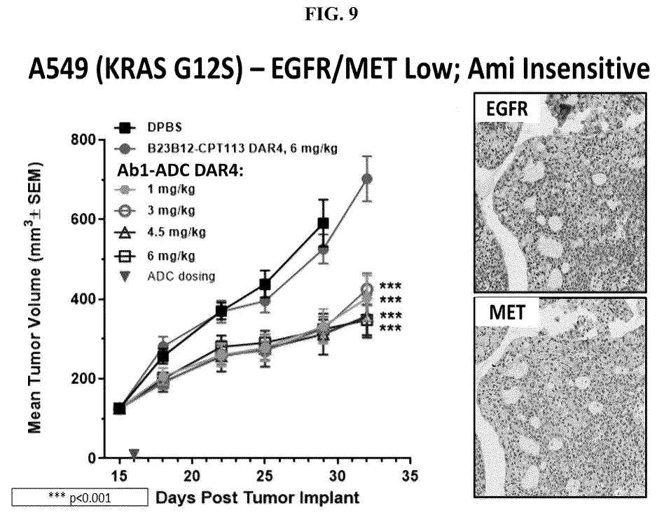

The antitumor efficacy of Ab1-ADC was evaluated in a second KRAS mutant (G12S) lung cancer xenograft model, A549. Athymic nude mice bearing established SC A549 xenografts were dosed once IV with Ab1-ADC at 1, 3, 4.5, and 6 mg/kg or isotype-ADC (B23B12-CPT113, DAR 4) at 6 mg/kg. DPBS was used as a vehicle control. Treatment with Ab1-ADC resulted in ΔTGI of 52%, 48%, 60%, and 61%, respectively (p<0.001; FIG. 9, Table 7) as compared to isotype-ADC on Day 32 post tumor implantation. The decreased efficacy of Ab1-ADC observed in A549 was consistent with the lower expression of EGFR and MET observed by IHC and the intrinsic sensitivity of A549 to the cytotoxic payload (FIG. 9, Table 2). Indeed, compared to EBC-1, H1975, and H1373, representative IHC images from A549 xenograft tumors demonstrated much less intense staining for both EGFR and MET. While statistically significant ΔTGI (p<0.001) was observed upon treatment with Ab1-ADC at 1, 3, 4.5, and 6 mg/kg and 1 PR was observed in the 4.5 mg/kg group, no treatment groups reached biologically significant efficacy (60% TGI) and therefore a MED was not established.

TABLE 6
Tumor Growth Inhibition in MET & EGFR Independent
(KRAS Mutant) NSCLC Xenografts (H1373 (KRAS G12C) -
EGFR/MET High; Ami Insensitive) by of Exemplary
Bispecific Anti-EGFR/c-Met Antibody-Drug Conjugate

Day 25 vs Isotype-ADC

| Ab1-ADC Dose | % TGI | %ΔTGI | % TR, PR/CR |
|---|---|---|---|
| 1 mg/kg | — | — | — |
| 3 mg/kg | 73 | 88 | 0, 5/0 |
| 6 mg/kg | 91 | 109 | 44, 9/0 |

TABLE 7
Tumor Growth Inhibition in MET & EGFR Independent
(KRAS Mutant) NSCLC Xenografts (A549 (KRAS G12S) -
EGFR/MET Low; Ami Insensitive) by Exemplary Bispecific
Anti-EGFR/c-Met Antibody-Drug Conjugate

Day 32 vs Isotype-ADC

| Ab1-ADC Dose | % TGI | %ΔTGI | % TR, PR/CR |
|---|---|---|---|
| 1 mg/kg | 43 | 52 | — |
| 3 mg/kg | 40 | 48 | — |
| 4.5 mg/kg | 49 | 60 | 0, 1/0 |
| 6 mg/kg | 50 | 61 | — |

### Example 8. Efficacy of Ab1-ADC in a Colorectal Cancer (CRC) Xenograft Model

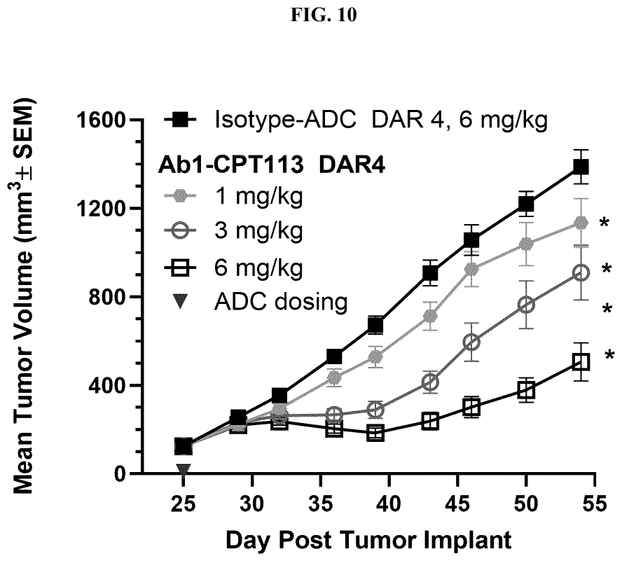

Antitumor efficacy of Ab1-ADC was evaluated in WT EGFR, KRAS mutant (G12D) CRC PDX model, CR3262, previously known to be resistant to amivantamab. Balb/c nude mice bearing established SC PDX model, CR3262, were dosed once IV with Ab1-ADC at 1, 3, and 6 mg/kg or isotype-ADC (B23B12-CPT113, DAR4) at 6 mg/kg. Briefly, tumor fragments were implanted on Day 0, followed by treatment on Day 25. Significant antitumor efficacy was observed with Ab1-ADC treatment at 1, 3, and 6 mg/kg as assessed by change in mean tumor burden on Day 54 post tumor implantation (p<0.05; FIG. 10, Table 8), with ΔTGI of 20%, 38%, and 70% respectively, compared to isotype-ADC-treated control.

TABLE 8
Tumor Growth Inhibition in KRAS mutant CRC PDX CR3262
(WT EGFR; Ami Insensitive) by Exemplary Bispecific
Anti-EGFR/c-Met Antibody-Drug Conjugate.

Day 54 vs Isotype-ADC

| Ab1-ADC Dose | % TGI | %ΔTGI | % TR, PR/CR |
|---|---|---|---|
| 1 mg/kg | 18 | 20 | — |
| 3 mg/kg | 35 | 38 | — |
| 6 mg/kg | 64 | 70 | — |

### Example 9. Molecular Profile of Exemplary Bispecific Anti-EGFR/c-Met Antibody-Drug Conjugate

Table 9 shows the molecular profile of exemplary bispecific anti-EGFR/c-Met antibody-drug conjugate.

TABLE 9
Selected Molecular Profile of Exemplary Bispecific
Anti-EGFR/c-Met Antibody-Drug Conjugate

| | |
|---|---|
| MET Affinity | 0.06 nM |
| EGFR Affinity | 1.15 nM |
| Biophysical | Fab Tm > 70° C. |
| Properties | Minimal aggregation |
| | No nonspecific binding |
| | Serum Stable |
| ADC Properties | Dual maleimide disulfide rebridging |
| | conjugation |
| | Topoisomerase inhibitor payload (DAR4) |
| Functional Profile | OR binding and internalization profile |
| | Potent in vitro cytotoxicity in EGFR × MET+ cell |
| | lines (EC50 < 1 nM) |
| | Efficacy in disease EGFR × MET+ in vivo |
| | models, including disease relevant tissue |
| | models |
| Tolerability | Tolerated in cyno at doses to enable a minimum |
| | TI ≥ 3 |
| Conformational | Tm1/Tm2 |
| stability (DSC) | 66.4° C./73.1° C. |
| Serum stability (SEC- | 95.4% monomer, 2.7% HMW (Δ 2.3%), 1.9% |
| FDS, % monomer, 7 | LMW (Δ 0.8%) |
| d, 37° C.) | |
| DAR by LC-MS | Main species 4.0 |
| Serum stability of | Minimal change in DAR after 7 days |
| payload linker | |
| (7 d, 37° C.) | |
| Stress analysis | Payload Linker: minimal changes in DAR or |
| | payload integrity. |

### Example 10. Synthesis of Ab1-ADC

Synthesis of the Linker-Payload (I) depicted below:

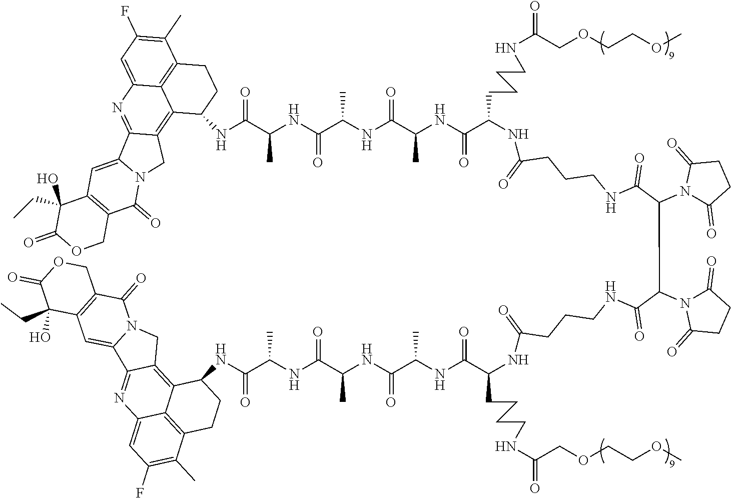

### Overall Synthetic Scheme of Linker Payload:

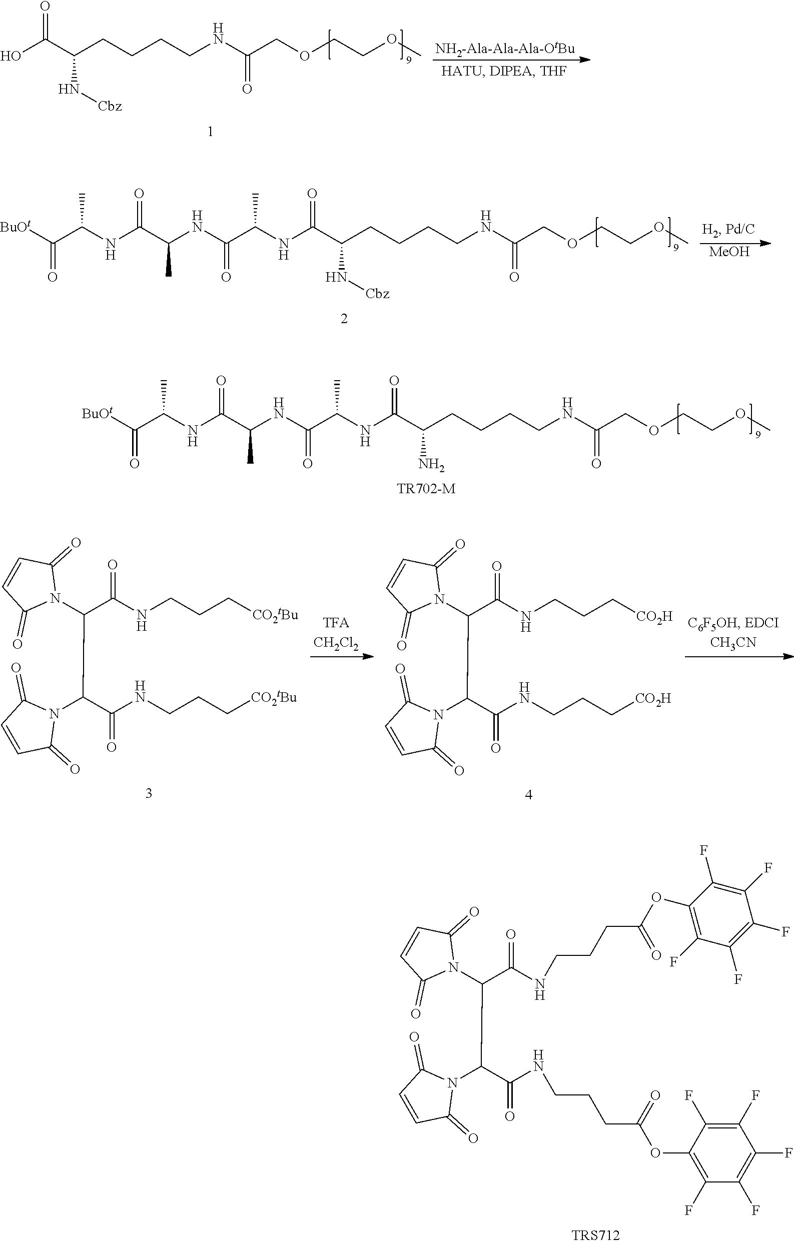

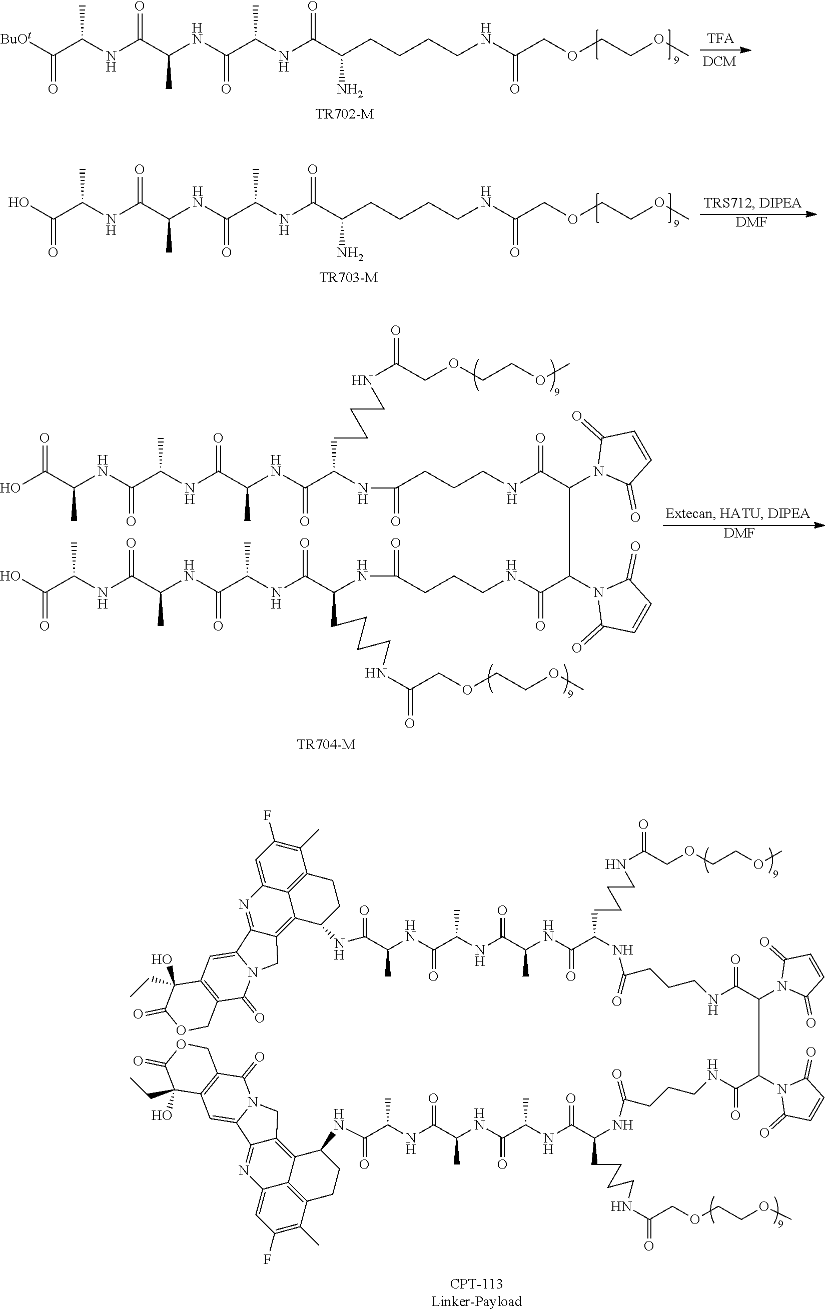

### General Procedures

All solvents and reagents were commercially obtained and used without purification. Mass spectrometry analysis was performed on a Waters Xevo G21 quadrupole time-of-flight (QTOF) system in positive ESI mode.

### Procedure for Preparation of Compound 1

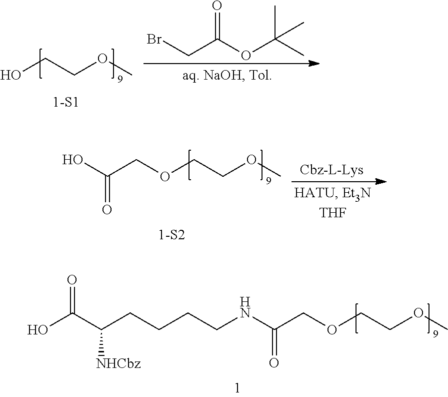

To a solution of 1-S1 (20.0 g, 46.7 mmol) in toluene (150 mL) was added NaOH (aq) (121 g, 1.52 mol, 50 wt %) dropwise at 0-10° C., followed by tert-butyl 2-bromoacetate (18.2 g, 93.3 mmol, 13.8 mL) at 0-10° C. over 30 min. The mixture was stirred for 14 hours and quenched with H₂O (250 mL). The phases were separated, and the aqueous phase was adjusted to pH 1-2 using concentrated HC1 (115 mL), and then extracted with dichloromethane (DCM) three times (200 mL). The organic layers were washed with brine (200 mL) and dried over Na₂SO₄, filtered and concentrated under reduced pressure to give 1-S2 (23.0 g, crude) as a light-yellow oil, which was directly used in the next step without further purification.

To a solution of compound 1-S2 (3.30 g, 6.78 mmol) and Cbz-L-Lys-OH (1.90 g, 6.78 mmol) in tetrahydrofuran (THF) (50 mL), hexafluorophosphate azabenzotriazole tetramethyl uronium (HATU) (3.81 g, 10.0 mmol) and triethylamine (1.94 ml, 13.4 mmol) were added. The reaction was stirred at RT until completion, as indicated by LC-MS. The solvent was removed, and the residue was poured into water (100 mL), then extracted with DCM (3×50 mL). The combined organic phases were washed with water (50 mL), saturated sodium bicarbonate (50 mL), 2 N HCl (50 mL), and brine (50 mL), dried over sodium sulfate, filtered and concentrated, purified by silica gel column (DCM/MeOH=100/0 to 10/1), to give compound 1 (3.7 g, 59% yield).

### Procedure for Preparation of Compound 2

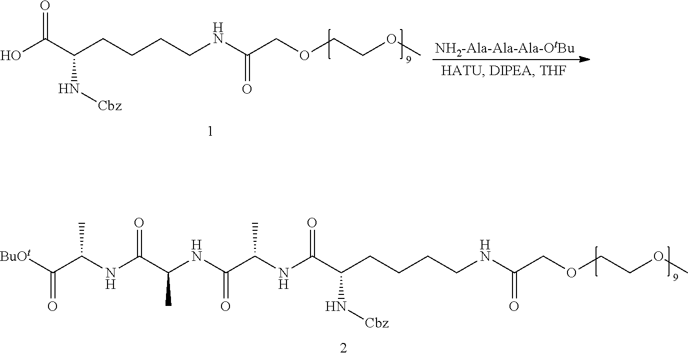

To a solution of(S)-37-(((benzyloxy) carbonyl)amino)-31-oxo-2,5,8,11,14,17,20,23,26,29-decaoxa-32-azaoctatriacontan-38-oic acid (compound 1, 16.94 g, 22.6 mmol) in THF (160 mL), HATU was added (10.32 g, 27.1 mmol). The mixture was stirred for 10 minutes, then tert-butyl L-alanyl-L-alanyl-L-alaninate (7.15 g, 24.9 mmol) was added, followed by diisopropylethylamine (DIPEA) (7.31 g, 56.5 mmol). The reaction was stirred for 2 hours and concentrated under vacuum, then diluted with water and extracted with DCM. The combined organic layers were washed with 5% Na₂CO₃, water, 2 N HCl, and water; dried over Na₂SO4; and concentrated under vacuum. The crude reaction was purified by flash column chromatography (silica gel, 5%-15% methanol [MeOH]/DCM) to afford compound 2 (20.62 g, 89% yield).

### Procedure for Preparation of Compound TR702-M

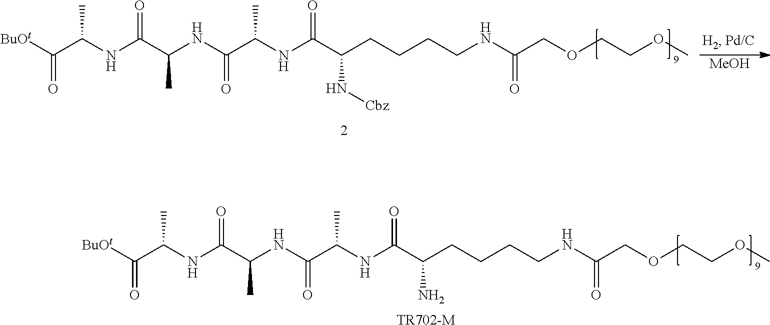

To a solution of compound 2 (20.62 g, 20.2 mmol) in MeOH (40 mL) at room temperature palladium on carbon (Pd/C) was added (2 g, 10 wt %). The reaction flask was evacuated and back-filled with H₂ three times, and the mixture was stirred at room temperature for 2 hours under a H₂ balloon before being filtered through a pad of celite. The filtrate was concentrated under vacuum to afford compound TR702-M (16.41 g, 92% yield), which was used for the next step without further purification.

### Procedure for Preparation of TR703-M

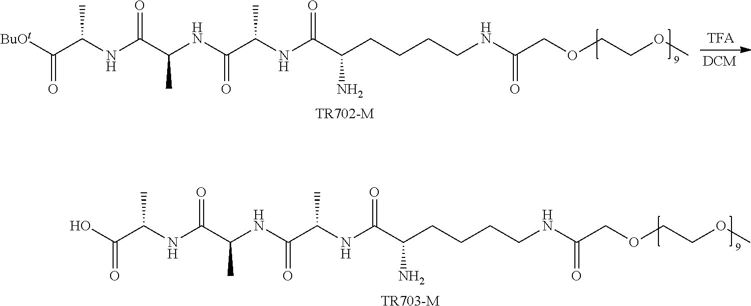

To a solution of TR702-M (20.73 g, 23.45 mmol) in DCM (145 mL), trifluoroacetic acid (TFA) (145 mL) was added dropwise at 20° C. The reaction mixture was allowed to warm to room temperature (RT) and stirred for 2 hours, then concentrated under vacuum to afford TR703-M (52.59 g) as a yellow oil which was and used for the next step without further purification.

### Procedure for Preparation of Compound 3

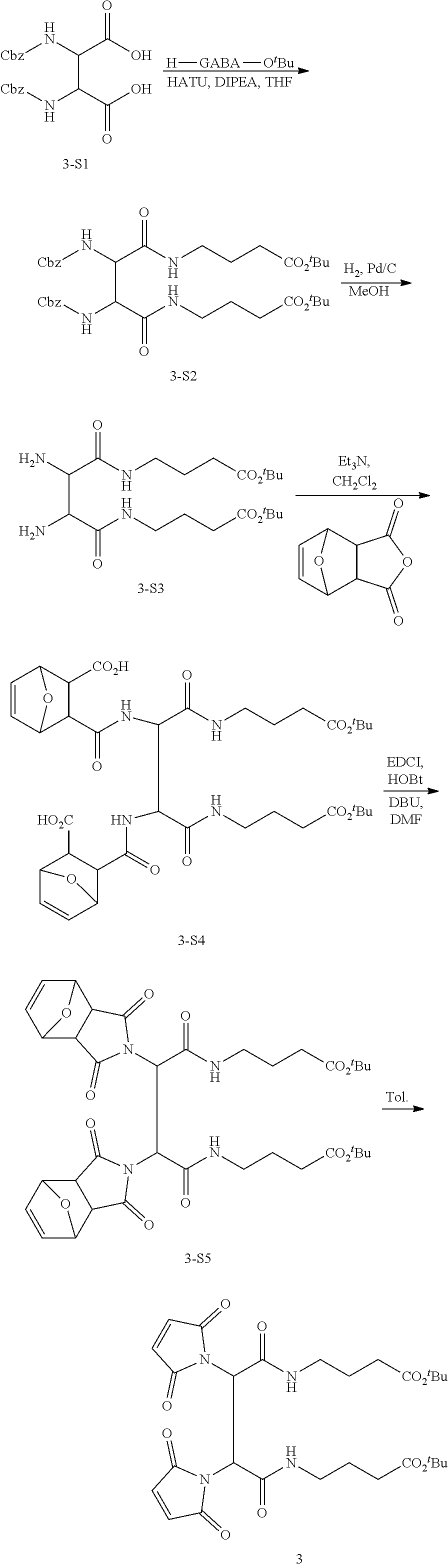

To a solution of compound 3-S1 (10 g, 28.7 mmol) and tert-butyl aminobutyrate hydrochloride (11.2 g, 57.4 mol) in THF (200 mL) were added HATU (32.8 g, 86.3 mmol) and DIPEA (19 mL, 115 mmol), and the reaction was stirred at RT overnight, diluted with water (400 mL), stirred for 10 minutes, filtered, and dried in an oven to give a white solid (17.7 g, >100% yield), which was used for the next step without any purification.

To a solution of 3-S2 (16 g, 22.9 mmol) in MeOH (200 mL) at room temperature was added Pd/C (2.0 g, 10 wt %). The reaction flask was evacuated and back-filled with hydrogen, heated to 60° C., and stirred until completion of the reaction. The mixture was filtered through a pad of celite. The filtrate was concentrated under vacuum to afford compound 3-S3, which was used for the next step without any purification.

Compound 3-S3 (9.8 g, 22.8 mmol) was dissolved in DCM (200 mL), and exo-3,6-epoxy-1,2,3,6-tetrahydrophthalic anhydride (7.5 g, 45.5 mmol) and triethylamine (6.3 ml, 45.5 mmol) were added. The reaction was stirred at RT until completion, and then concentrated to dryness to give a white foamy solid (17.3 g, 100% yield).

To a solution of compound 3-S4 (17.3 g, 22.7 mmol) dissolved in dimethylformamide (DMF) (300 mL), were added 1-Ethyl-3-(3-dimethylaminopropyl-carbodiimide hydrochloride (EDCI) (13 g, 68.2 mmol), 1-Hydroxybenzotriazole (HOBt) (9.2 g, 68.2 mmol), and 1,8-Diazabicyclo[5.4.0]undec-7-ene (DBU) (10.4 g, 68.2 mmol) slowly. The mixture was heated to 60° C. and stirred for 5 hours, cooled to RT, poured into water (1 L), and filtered to give a white solid (8.2 g, 49% yield).

Compound 3-S5 (8.2 g, 11.2 mmol) was dissolved in a mixture of toluene (80 mL) and DMF (80 mL), heated to 120° C., and stirred under reflux for 4 hours. The reaction was then cooled to RT and stirred for 30 minutes. A white solid precipitated which was then collected by filtration and dried to give the desired product 3 (3.0 g, 45% yield).

### Procedure for Preparation of Compound 4

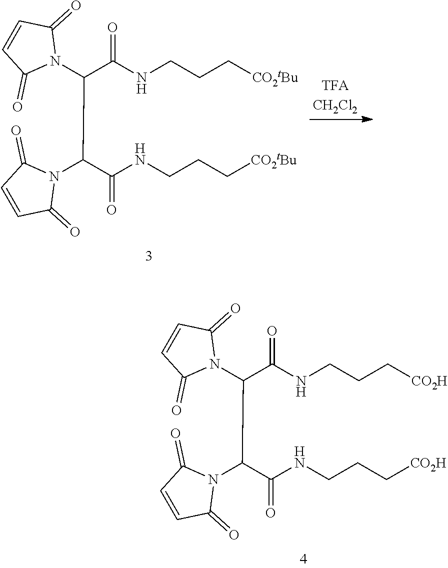 [SMILES: CC(C)(C)OC(=O)CCCNC(=O)C(C(C(=O)NCCCC(=O)OC(C)(C)C)N1C(=O)C=CC1=O)N1C(=O)C=CC1=O ; O=C(O)CCCNC(=O)C(C(C(=O)NCCCC(=O)O)N1C(=O)C=CC1=O)N1C(=O)C=CC1=O]

A solution of compound 3 (8.50 g, 14.4 mmol) in DCM (25 mL) and TFA (25 mL) was stirred for 5 hours and then concentrated under vacuum. The mixture was precipitated in hexanes/ethyl acetate (EtOAc) (1:1, 150 mL) and filtered to provide compound 4 (6.70 g, 97% yield) as a white solid.

### Procedure for Preparation of TRS712

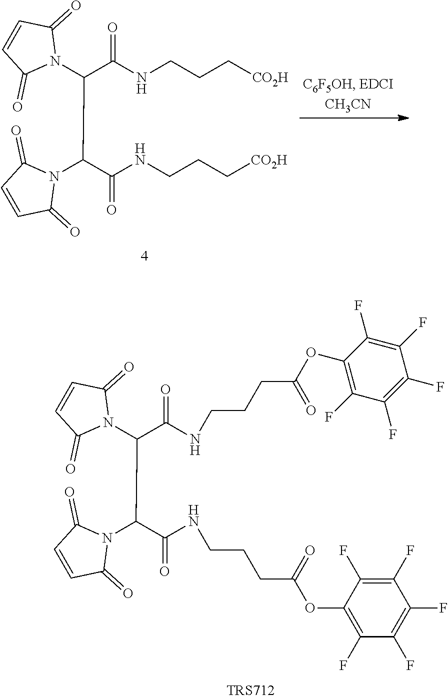 [SMILES: O=C(O)CCCNC(=O)C(C(C(=O)NCCCC(=O)O)N1C(=O)C=CC1=O)N1C(=O)C=CC1=O ; O=C(CCCNC(=O)C(C(C(=O)NCCCC(=O)Oc1c(F)c(F)c(F)c(F)c1F)N1C(=O)C=CC1=O)N1C(=O)C=CC1=O)Oc1c(F)c(F)c(F)c(F)c1F]

To a solution of compound 4 (2.00 g, 4.18 mmol) in CH₃CN (40 mL), 2,3,4,5,6-pentafluorophenol (2.31 g, 12.5 mmol) and EDCI (2.40 g, 12.5 mmol) were added. The reaction mixture was stirred for 6 hours and concentrated under vacuum. The residue was triturated with DCM (20 mL) and filtered to provide compound TRS712 (2.0 g, 59% yield).

### Procedure for Preparation of TR704-M

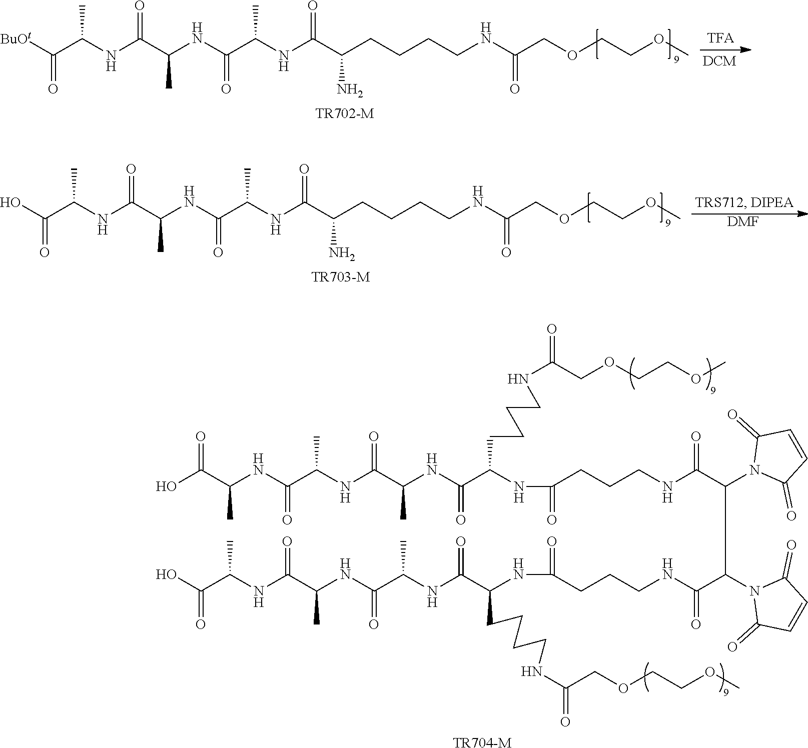

TRS712 was added to a solution of TR703-M (12.2 g, 14.72 mmol) in anhydrous N,N-DMF (180 mL) (5.68 g, 7.01 mmol) at 10° C. followed by DIPEA (31.9 mL, 177.6 mmol) and the mixture was stirred at room temperature for 3 hours until conversion was completed. The reaction mixture was added dropwise into stirring methyl tert-butyl ether (2 L). A white precipitate was formed and collected by filtration, and then dissolved in DMF (158.56 g, yield 90.5%), which was used for the next step without further purification.

### Procedure for Preparation of Linker-Payload

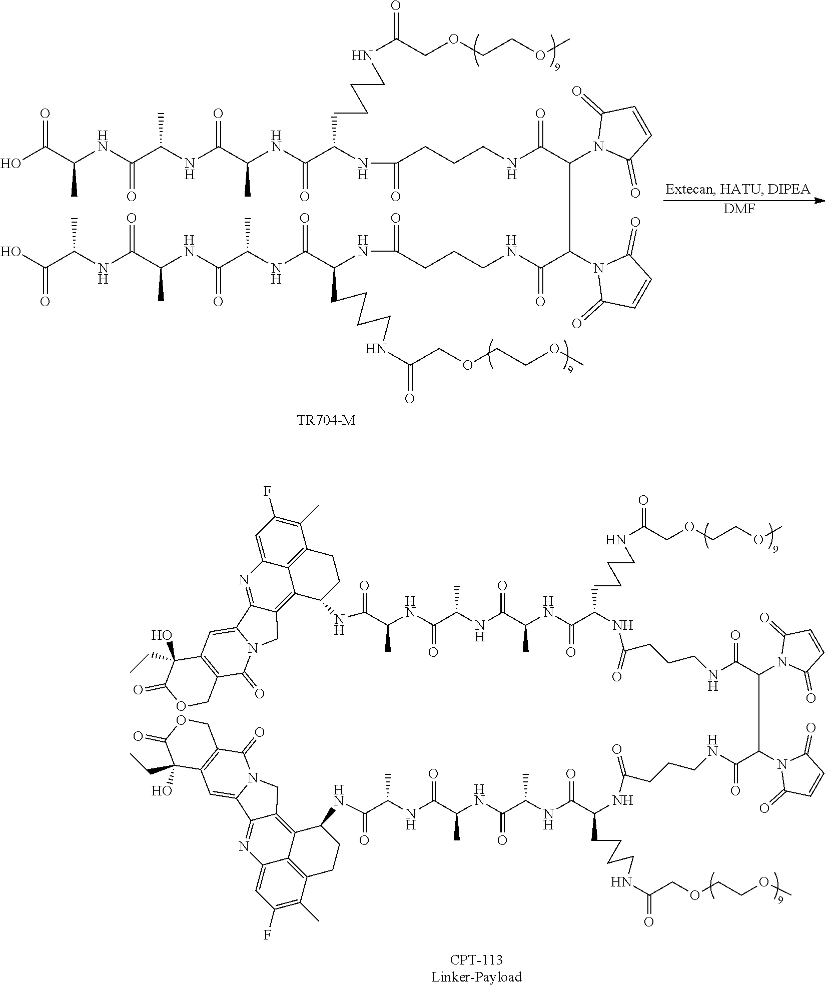

To a solution of TR704-M (3.68 g, 1.753 mmol) in DMF, HATU was added (1.73 g, 4.56 mmol). The mixture was stirred at room temperature for 20 minutes and then a mixture of exatecan mesylate (2.33 g, 4.4 mmol) and DIPEA (1.7 mL, 9.6 mmol) in DMF (25 mL) was added. The reaction mixture was stirred for 30 minutes and concentrated to afford the crude Linker-Payload. The crude material was purified by prep-HPLC (C18, phase A CH₃CN, phase B 0.2% TFA in H₂O, 70 min, 90%-20% B, flow rate 1.2 L/min), the fractions were collected and concentrated, and triturated with methyl tert-butyl ether (MTBE). The solid was collected and dried to give Linker-Payload (CPT-113, I) (3.90 g, 57.3% yield over three steps).

### Characterization of Linker-Payload (CPT-113, I)

The structure of Linker-Payload was confirmed by 1H NMR and infrared (IR) spectroscopy. Purity was assessed to be 94% by reverse phase high performance liquid chromatography (RP-HPLC) based on area of the main peak.

### Conjugation of Linker-Payload to Amivantamab Antibody:

The final conjugate Ab1-ADC was produced by coupling amivantamab antibody with linker-payload (CPT-113, I) with a DARs of 4, 6 and 8. The reaction was performed under conditions to drive preferential conjugation to the interchain cysteines at the heavy chain (HC)-light chain (LC) interface and to limit conjugation to the hinge disulfides.

The antibody-drug conjugate was produced by reacting the bispecific antibody (amivantamab) with CPT113, a bis-maleimide exatecan linker-payload molecule with an enzyme-cleavable tri-Alanine linker and Lysine (PEG10) spacer. The reaction was performed under conditions that drive preferential conjugation at the HC-LC disulfides with limited conjugation at the hinge disulfide cysteines.

3.4 grams of amivantamab (161 mg/mL) was thawed to room temperature, protected from light, and was diluted to 8 mg/mL by addition of 10 mM histidine buffer, pH 6.9 in a 1 L PETG bottle. Solution was mixed at 50 rpm. pH was measured and confirmed to fall in the target range (6.5±0.2). After cooling to 2-8° C. in an incubator, the following solutions were added in sequence with pipettes: (1) 50 mM Zn11 (FIG. 2) in DMA (1379 μL), was added slowly to the mixing antibody solution and stirred for 10-30 min. (2) 100 mM TCEP aqueous solution (574.6 μL) was added to the mixture and stirred at 2~8° C. for 15-17 hr.

### Structure of Zn11

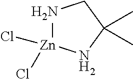

A solution of CPT113 in DMA (90 mg/mL, 2849 μL) was added slowly to the mixture and stirred for 2~4 h. The reaction was then quenched by sequential addition of aqueous 100 mM L-cysteine (1724 μL), 100 mM DHAA in DMA (6896 μL), and aqueous 100 mM EDTA (1379 μL). Mixing was continued for 1-2 h followed by filtration through a 0.22 μm filter. The quenched and filtered reaction mixture was stored at 2~8° C. protected from light until purification.

Ab1-ADC was purified by cation exchange chromatography (CEX). A column with 2.6 cm inner diameter×30 cm length was packed with Capto S impact resin (column bed height ~11.8 cm, column volume (CV) 62.5 mL; retention time 5.2 min, volume flow rate 12 mL/min, linear flow rate: 136.2 cm/h). The resin was washed with 5 CV of water, 5 CV of 0.5 M NaOH and 5 CV of elution buffer, and 5 CV of wash buffer, and 5 CV of load/equilibration buffer (20 mM L-histidine pH 5.0).

The reaction mixture was diluted 2× with load buffer, and then pH adjusted to 5.0±0.2 with 1 M acetic acid. Sample was warmed to at least 15° C. and then loaded onto the column at 12 mL/min on an AKTA Avant instrument. Column was equilibrated with 5 CVs of equilibration buffer then washed with 7 CVs of wash buffer (20 mM L-histidine, pH 5.5).

One of skill in the art, can synthesize the single maleimide ADC (Ab1-ADC2) through a similar synthetic scheme and procedure.

Ab1-ADC2 has the following structure:

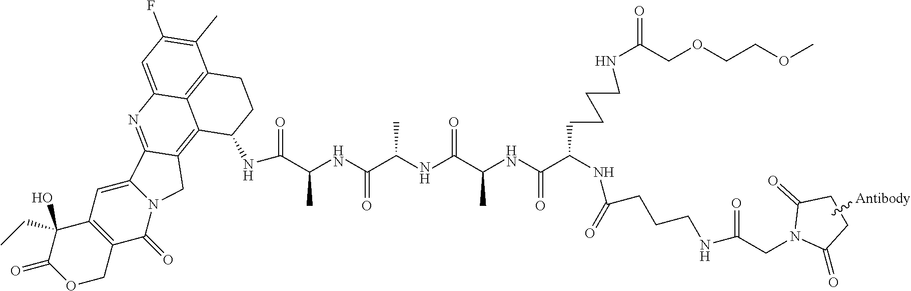

### Synthesis of the Hydrolyzed Form of Ab1-ADC

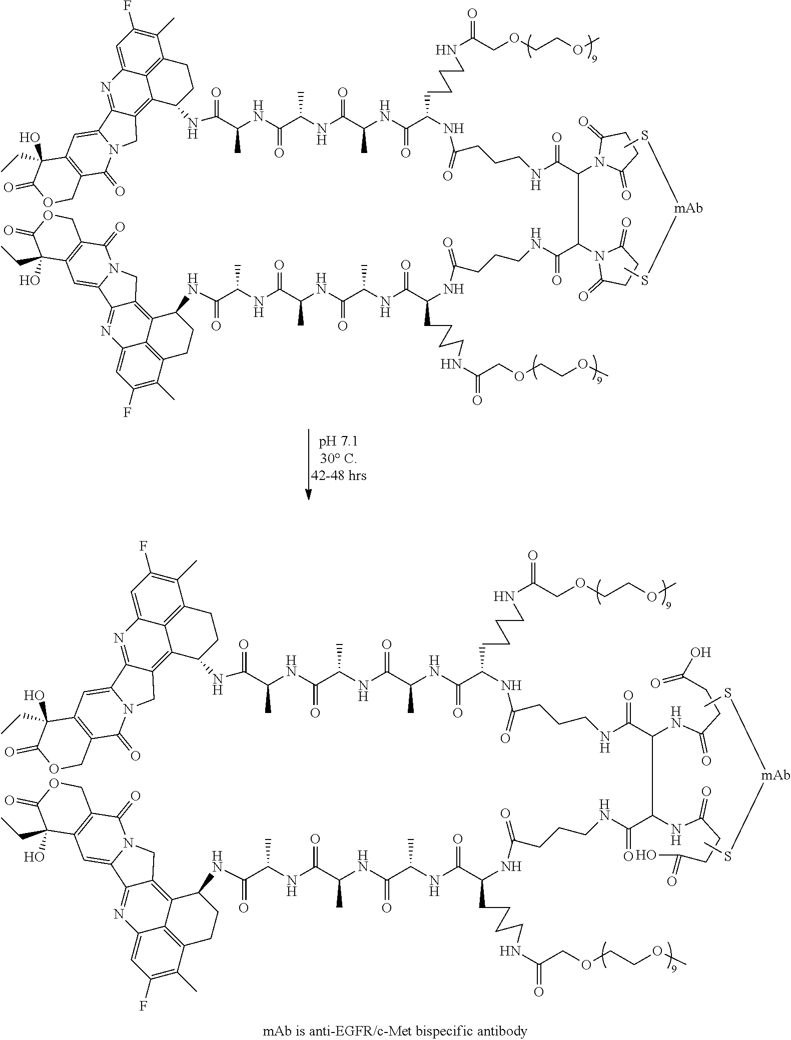

To increase stability of the connection between the antibody and the linker-payload, the purified ADC was incubated in conditions that resulted in opening of the succinimide ring. A mild method of hydrolysis of the succinimide-thioether ring results in ring opening to the hydrolyzed structure. The succinimide thioether ring of the ADC solution was further subjected to buffer exchange, under adjusted to pH 7.1 by the addition of 400 mM Na₂HPO₄ and then stirred at 30° C. for 42-48 hr. The resulting solution was diafiltered into formulation buffer, filter sterilized, and characterized to determine DAR, concentration, % monomer, etc. using standard analytical techniques.

### Example 11. PK Parameters of Ab1-ADC with DARs of 4, 6 and 8 and Ab1-ADC2 with a DAR of 8

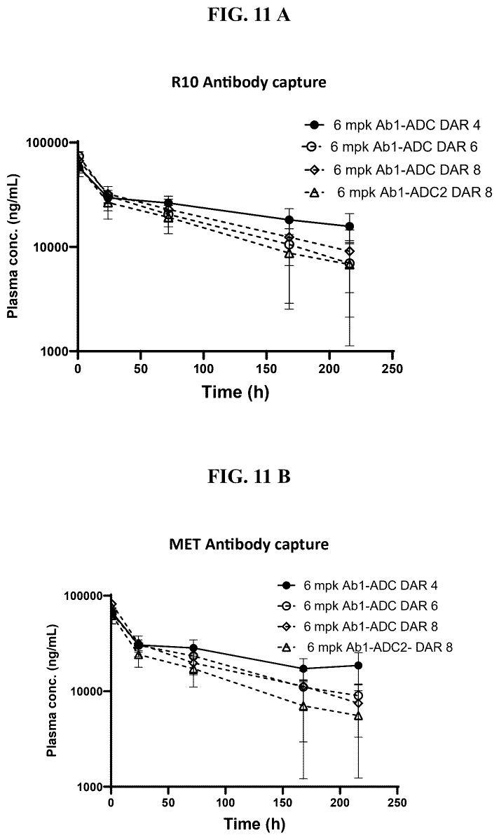

A single-dose pharmacokinetic study was conducted in non-tumor-bearing mice to evaluate the disposition of antibody-drug conjugates (ADCs) with varying drug-to-antibody ratios (DAR). Non-tumor bearing nude mice were dosed once IV with Ab1-ADC with a DAR of 4, 6 and 8 or Ab1-ADC2 with a DAR 8 at 6 mg/kg. Plasma was collected at 1 hr, 24 hr, 72 hr, 7d and 9d post dose for exposure. Plasma concentrations of ADC were analyzed by Immunocapture-LC-MS/MS following immune capture and papain digestion (ADC) or trypsin digestion (total antibody). Two capture reagents, R10 (anti-IgG Fc antibody, FIG. 11A) and cMET (FIG. 11B) were used for capture. PK measurements were performed using either R10 (anti-Fc) or cMET as the capture reagent. Further, PK parameters including Cmax, AUC, clearance (C1) and half life (HL_lambda_z) were assessed by non-compartmental analysis (Table 10).

Both capture reagents resulted in similar PK profiles and similar conclusions. This analysis demonstrated a relationship between DAR and the PK profile of Ab1-ADC. The increase in DAR was directly associated with an increase in the clearance of the ADC in vivo, with DAR 4 Ab1-ADC demonstrating the slowest clearance rate compared with DAR 6 and DAR 8. Interestingly, Ab1-ADC exhibited a more favorable PK profile when compared to Ab1-ADC2 at the same DAR of 8, likely due to the dual maleimide conjugation chemistry of Ab1-ADC. Taken together, these data demonstrate that Ab1-ADC DAR 4 has the most favorable PK profile.

TABLE 10
PK parameters measured from non-tumor bearing nude mice dose with 6 mg/kg
of either Ab1-ADC with DARs of 4, 6 and 8, or Ab1-ADC2 at a DAR of 8.

| | DoseAmt | Cmax | AUClast | AUCINF_pred | AUC_% | CL_pred | Vz_pred | HL_Lambda_z | |
|---|---|---|---|---|---|---|---|---|---|
| Group | (mg/kg) | (ug/mL) | (day*ug/mL) | (day*ug/mL) | Extrap_pred (%) | (mL/day/kg) | mL/kg | (day) | Rsq |
| Ab1-ADC DAR 4 R10 | 6 | 57.9 | 295.5 | 418.5 | 29.39 | 14.34 | 181.16 | 8.76 | 1 |
| Ab1-ADC DAR 4 CMET | 6 | 67.4 | 318.2 | 466.3 | 0.34 | 12.85 | 174.05 | 9.39 | 0.95 |
| Ab1-ADC DAR 6 R10 | 6 | 1.6 | 186.8 | 195.7 | 4.57 | 30.65 | 152.94 | 3.46 | 1 |
| Ab1-ADC DAR 6 CMET | 6 | 63.5 | 196.3 | 206 | 3.46 | 29.13 | 145.47 | 3.46 | 0.99 |
| Ab1-ADC DAR 8 R10 | 6 | 66.8 | 225.8 | 243.2 | 7.18 | 24.67 | 139.9 | 3.93 | 1 |
| Ab1-ADC DAR 8 CMET | 6 | 81.9 | 212.8 | 230.5 | 4.27 | 26.03 | 160.34 | 4.27 | 1 |
| Ab2-ADC DAR 8 R10 | 6 | 58.8 | 168.1 | 168.9 | 0.49 | 35.52 | 82.3 | 1.61 | 0.97 |
| Ab2-ADC DAR 8 CMET | 6 | 62.1 | 147.3 | 147.4 | 1.07 | 40.7 | 62.63 | 1.07 | 0.96 |

Increased DAR results in faster clearance of the Ab1-ADC resulting in a better developability profile. DAR 4 Ab1-ADC exhibits slow clearance when compared to the ADC having DAR of 6 or 8. All Ab1-ADCs exhibit good payload-linker in-vivo stability in mouse.

Those skilled in the art will appreciate that numerous changes and modifications can be made to the preferred embodiments disclosed herein and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

What is claimed is:

1. A bispecific anti-EGFR/c-Met antibody-drug conjugate (ADC), comprising
a bispecific anti-EGFR/c-Met antibody that comprises:
a first heavy chain (HC1) comprising a heavy chain complementarity determining region 1 (HCDR1) comprising the amino acid sequence of SEQ ID NO: 1, a HCDR2 comprising the amino acid sequence of SEQ ID NO: 2, and a HCDR3 comprising the amino acid sequence of SEQ ID NO: 3;
a first light chain (LC1) comprising a light chain complementarity determining region 1 (LCDR1) comprising the amino acid sequence of SEQ ID NO: 4, a LCDR2 comprising the amino acid sequence of SEQ ID NO: 5, and a LCDR3 comprising the amino acid sequence of SEQ ID NO: 6;
a second heavy chain (HC2) comprising a HCDR1 comprising the amino acid sequence of SEQ ID NO: 7, a HCDR2 comprising the amino acid sequence of SEQ ID NO: 8, and a HCDR3 comprising the amino acid sequence of SEQ ID NO: 9; and
a second light chain (LC2) comprising a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12; and wherein the bispecific anti-EGFR/c-Met antibody is connected to the linker-payload to (a) through the cysteine residues of said antibody to either carbon of the 5 membered succinimide ring of compound II or (b) through the cysteine residues of said antibody to either carbon of the ring opened compound III comprising the structures:

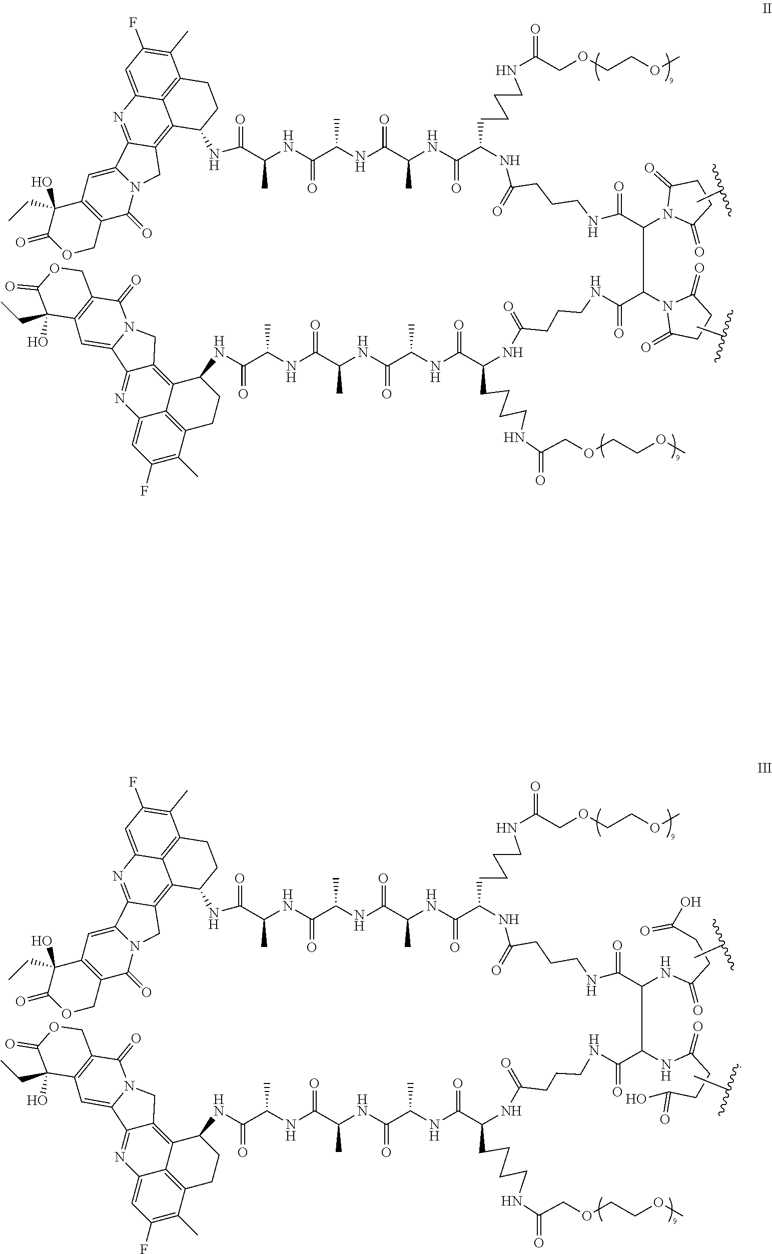

2. The bispecific anti-EGFR/c-Met ADC of claim 1, wherein the bispecific anti-EGFR/c-Met antibody is connected to the linker-payload through the cysteine residues of said antibody to either carbon of the 5 membered succinimide ring of compound II that comprises the structure:

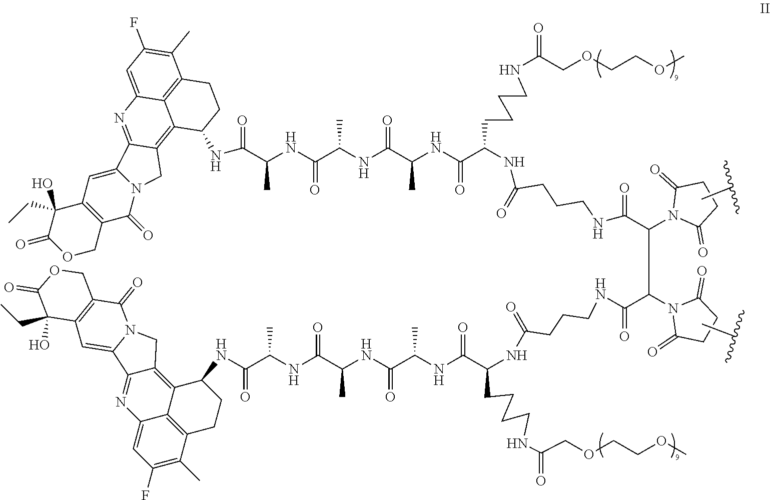

3. The bispecific anti-EGFR/c-Met ADC of claim 1, wherein the bispecific anti-EGFR/c-Met antibody is connected to the linker-payload through the cysteine residues of said antibody to either carbon of the ring opened compound III that comprises the structure:

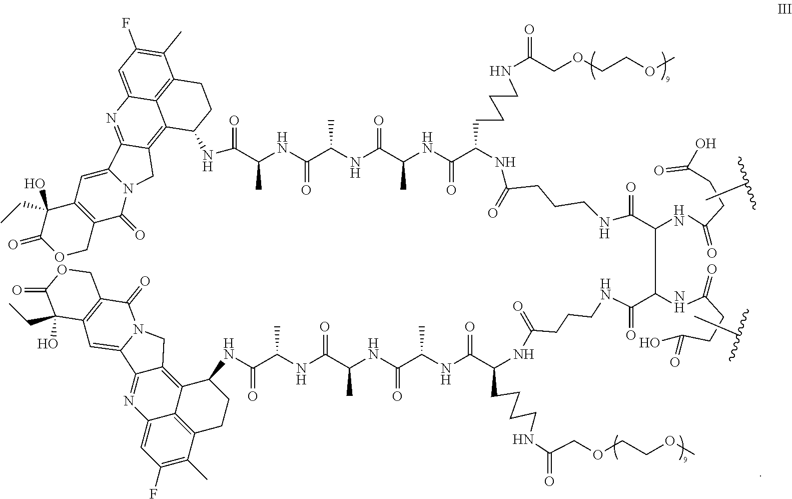

4. The bispecific anti-EGFR/c-Met ADC of any one of claim 1 or 2, wherein the bispecific anti-EGFR/c-Met ADC comprises Formula I:

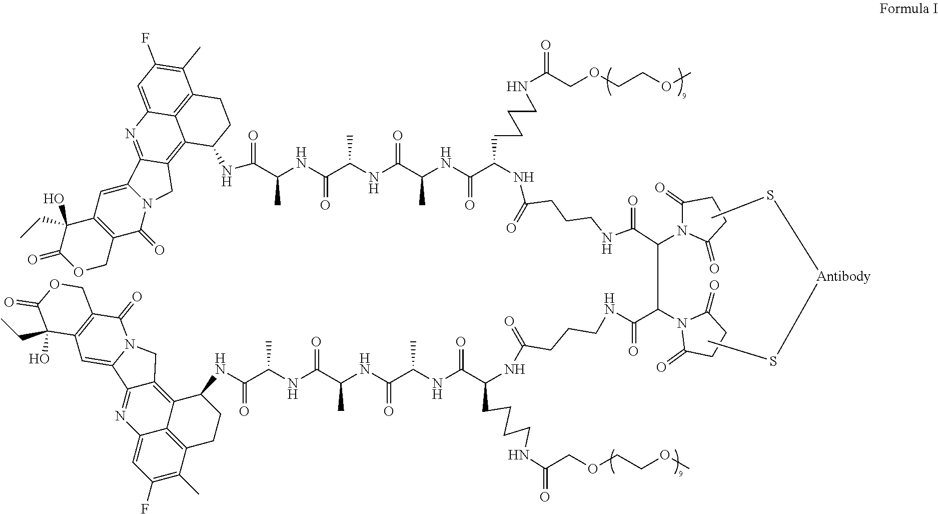

5. The bispecific anti-EGFR/c-Met ADC of any one of claim 1 or 3, wherein the bispecific anti-EGFR/c-Met ADC comprises Formula II:

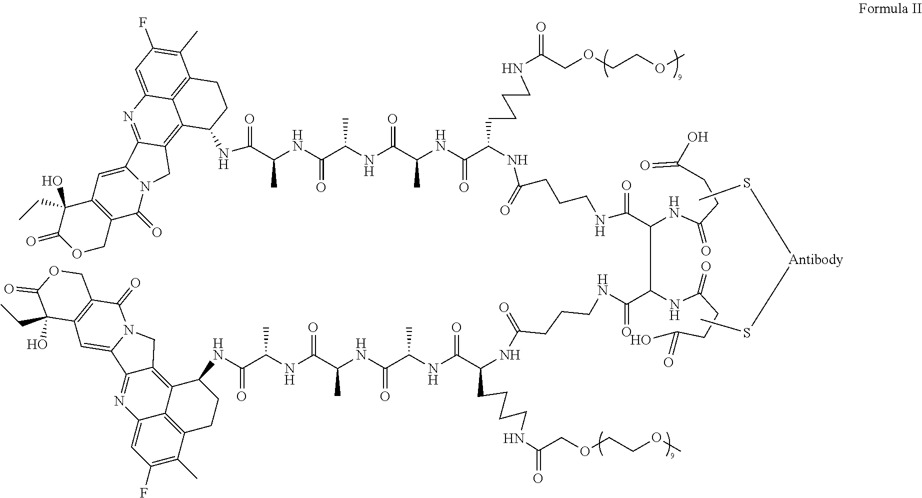

6. The bispecific anti-EGFR/c-Met ADC of any one of the previous claims, wherein:
the HC1 comprises a variable region (VH1) comprising the amino acid sequence of SEQ ID NO: 13;
the LC1 comprises a variable region (VL1) comprising the amino acid sequence of SEQ ID NO: 14;
the HC2 comprises a variable region (VH2) comprising the amino acid sequence of SEQ ID NO: 15; and
the LC2 comprises a variable region (VL2) comprising the amino acid sequence of SEQ ID NO: 16.

7. The bispecific anti-EGFR/c-Met ADC of any one of the previous claims, wherein the bispecific anti-EGFR/c-Met antibody is an IgG1 isotype.

8. The bispecific anti-EGFR/c-Met ADC of any one of the previous claims, wherein:
the HC1 comprises the amino acid sequence of SEQ ID NO: 17;
the LC1 comprises the amino acid sequence of SEQ ID NO: 18;
the HC2 comprises the amino acid sequence of SEQ ID NO: 19; and
the LC2 comprises the amino acid sequence of SEQ ID NO: 20.

9. The bispecific anti-EGFR/c-Met ADC of any one of the previous claims, wherein the bispecific anti-EGFR/c-Met antibody is amivantamab.

10. The bispecific anti-EGFR/c-Met ADC of any one of the previous claims 1, 2, 4, and 6-9, wherein the bispecific anti-EGFR/c-Met antibody drug conjugate comprises Formula I-A: 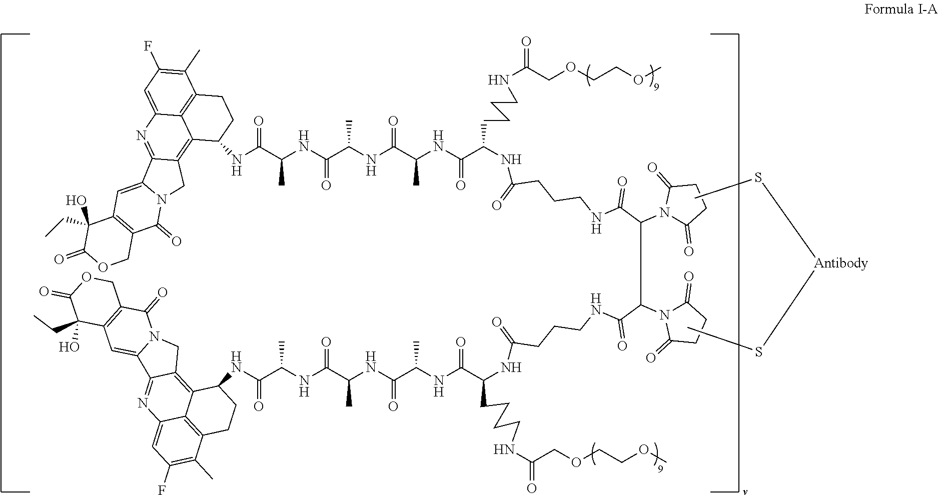
wherein v is 1, 2, 3 or 4.

11. The bispecific anti-EGFR/c-Met ADC of any one of the previous claims 1, 3, and 5-9, wherein the bispecific anti-EGFR/c-Met antibody drug conjugate comprises Formula II-A: 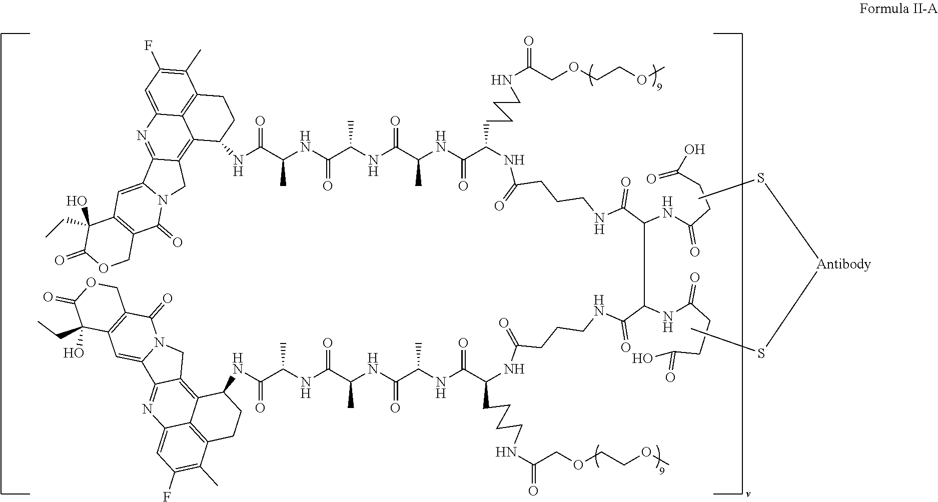
wherein v is 1, 2, 3 or 4.

12. The bispecific anti-EGFR/c-Met ADC of any one of the previous claims, comprising a drug-to-antibody ratio (DAR) of about 2 to about 8.

13. The bispecific anti-EGFR/c-Met ADC of any one of the previous claims, comprising a drug-to-antibody ratio (DAR) of about 4.

14. The bispecific anti-EGFR/c-Met ADC of claim 10, wherein the bispecific anti-EGFR/c-Met antibody drug conjugate comprises Formula I-A: 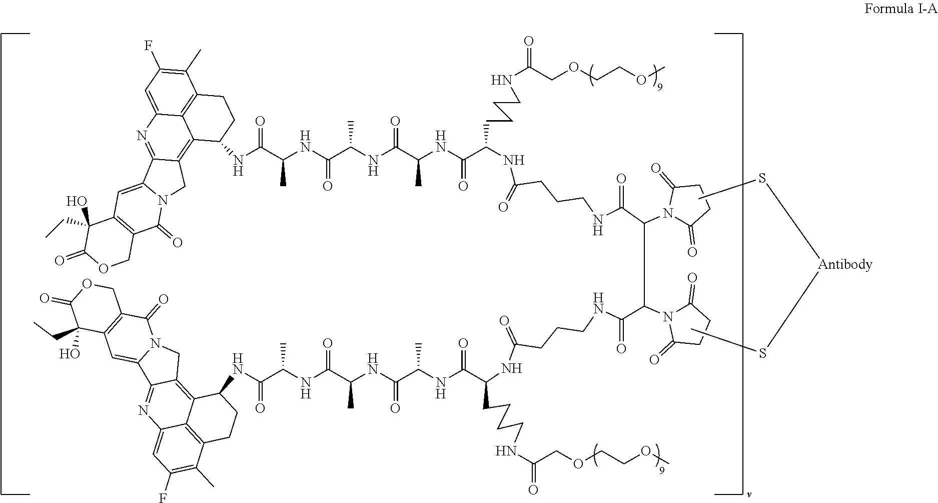
wherein v is 2.

15. The bispecific anti-EGFR/c-Met ADC of claim 11, wherein the bispecific anti-EGFR/c-Met antibody drug conjugate comprises Formula II-A: 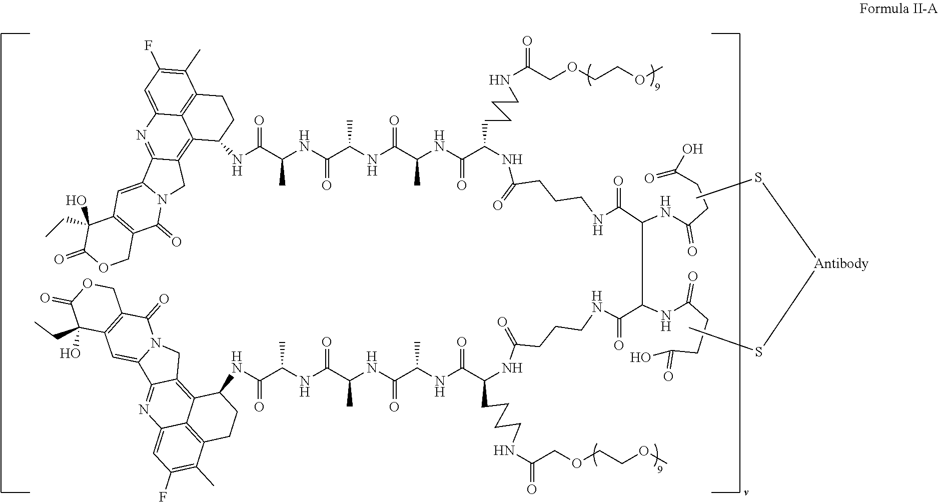
wherein v is 2.

16. A bispecific anti-EGFR/c-Met antibody drug conjugate (ADC) comprising a bispecific anti-EGFR/c-Met antibody that comprises:
a first heavy chain (HC1) comprising a heavy chain complementarity determining region 1 (HCDR1) comprising the amino acid sequence of SEQ ID NO: 1, a HCDR2 comprising the amino acid sequence of SEQ ID NO: 2, and a HCDR3 comprising the amino acid sequence of SEQ ID NO: 3; a first light chain (LC1) comprising a light chain complementarity determining region 1 (LCDR1) comprising the amino acid sequence of SEQ ID NO: 4, a LCDR2 comprising the amino acid sequence of SEQ ID NO: 5, and a LCDR3 comprising the amino acid sequence of SEQ ID NO: 6;
a second heavy chain (HC2) comprising a HCDR1 comprising the amino acid sequence of SEQ ID NO: 7, a HCDR2 comprising the amino acid sequence of SEQ ID NO: 8, and a HCDR3 comprising the amino acid sequence of SEQ ID NO: 9; and a second light chain (LC2) comprising a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12; and
wherein the bispecific anti-EGFR/c-Met antibody drug conjugate (ADC) comprises Formula I-A: 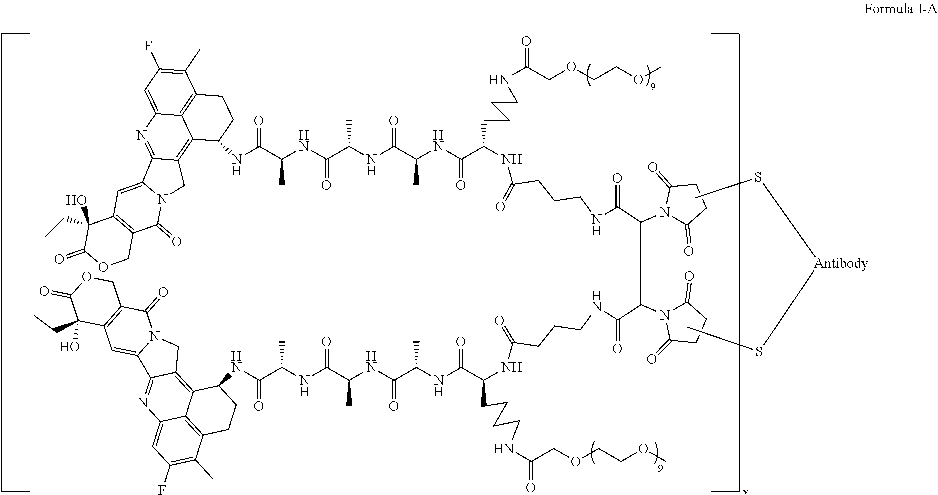
wherein the antibody is the anti-EGFR/c-Met bispecific antibody, v is 2; and
wherein the bispecific anti-EGFR/c-Met antibody drug conjugate (ADC) comprises conjugation at the interchain cysteine residues making up the HC-LC disulfide bonds resulting in a DAR of 4.

17. A bispecific anti-EGFR/c-Met antibody drug conjugate (ADC) comprising a bispecific anti-EGFR/c-Met antibody that comprises:
a HC1 comprising a variable region (VH1) comprising the amino acid sequence of SEQ ID NO: 13;
a LC1 comprising a variable region (VL1) comprising the amino acid sequence of SEQ ID NO: 14;
a HC2 comprising a variable region (VH2) comprising the amino acid sequence of SEQ ID NO: 15; and
a LC2 comprising a variable region (VL2) comprising the amino acid sequence of SEQ ID NO: 16; and
wherein the bispecific anti-EGFR/c-Met antibody drug conjugate (ADC) comprises Formula I-A: 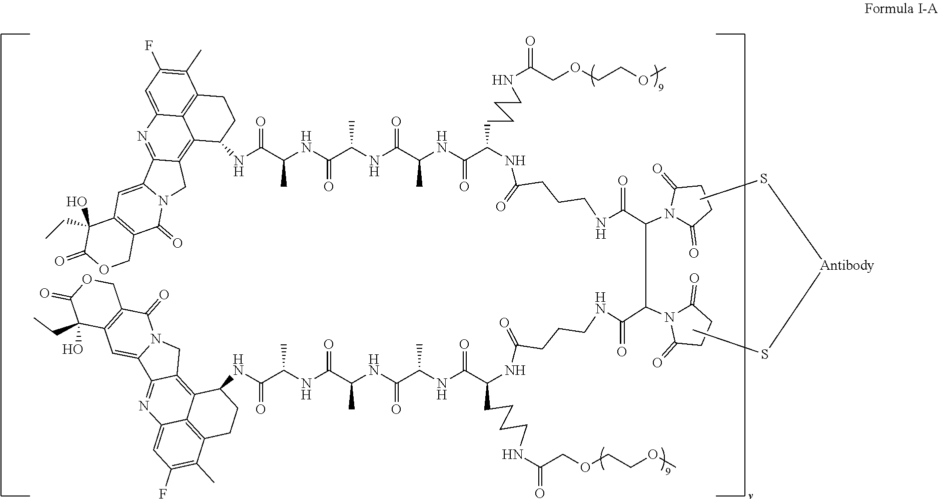
wherein the antibody is the anti-EGFR/c-Met bispecific antibody, v is 2; and
wherein the bispecific anti-EGFR/c-Met antibody drug conjugate (ADC) comprises conjugation at the interchain cysteine residues making up the HC-LC disulfide bonds resulting in a DAR of 4.

18. A bispecific anti-EGFR/c-Met antibody drug conjugate (ADC) comprising a bispecific anti-EGFR/c-Met antibody that comprises:
a HC1 comprising the amino acid sequence of SEQ ID NO: 17;
a LC1 comprising the amino acid sequence of SEQ ID NO: 18;
a HC2 comprising the amino acid sequence of SEQ ID NO: 19; and
a LC2 comprising the amino acid sequence of SEQ ID NO: 20; and
wherein the bispecific anti-EGFR/c-Met antibody drug conjugate (ADC) comprises Formula I-A: 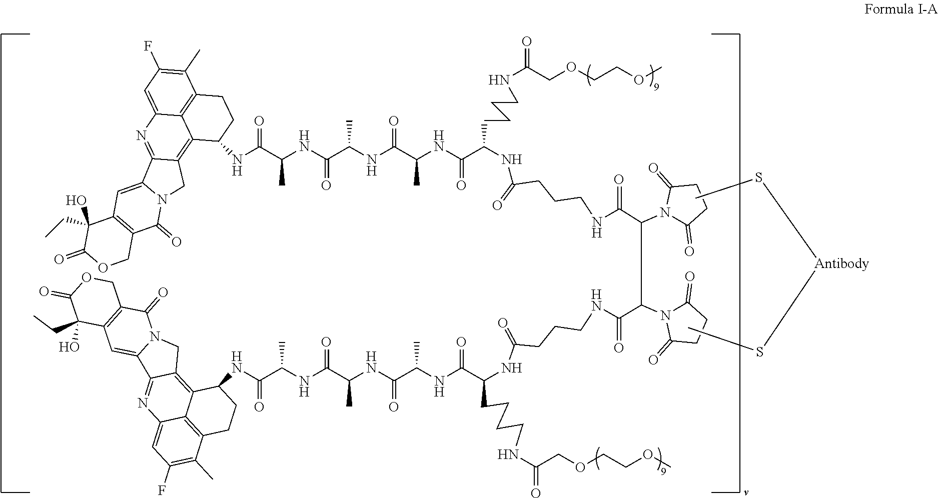
wherein the antibody is the anti-EGFR/c-Met bispecific antibody, v is 2; and
wherein the bispecific anti-EGFR/c-Met antibody drug conjugate (ADC) comprises conjugation at the interchain cysteine residues making up the HC-LC disulfide bonds resulting in a DAR of 4.

19. A bispecific anti-EGFR/c-Met antibody drug conjugate (ADC) comprising a bispecific anti-EGFR/c-Met antibody that comprises:
a first heavy chain (HC1) comprising a heavy chain complementarity determining region 1 (HCDR1) comprising the amino acid sequence of SEQ ID NO: 1, a HCDR2 comprising the amino acid sequence of SEQ ID NO: 2, and a HCDR3 comprising the amino acid sequence of SEQ ID NO: 3; a first light chain (LC1) comprising a light chain complementarity determining region 1 (LCDR1) comprising the amino acid sequence of SEQ ID NO: 4, a LCDR2 comprising the amino acid sequence of SEQ ID NO: 5, and a LCDR3 comprising the amino acid sequence of SEQ ID NO: 6;
a second heavy chain (HC2) comprising a HCDR1 comprising the amino acid sequence of SEQ ID NO: 7, a HCDR2 comprising the amino acid sequence of SEQ ID NO: 8, and a HCDR3 comprising the amino acid sequence of SEQ ID NO: 9; and a second light chain (LC2) comprising a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12; and
wherein the bispecific anti-EGFR/c-Met antibody drug conjugate (ADC) comprises Formula II-A: 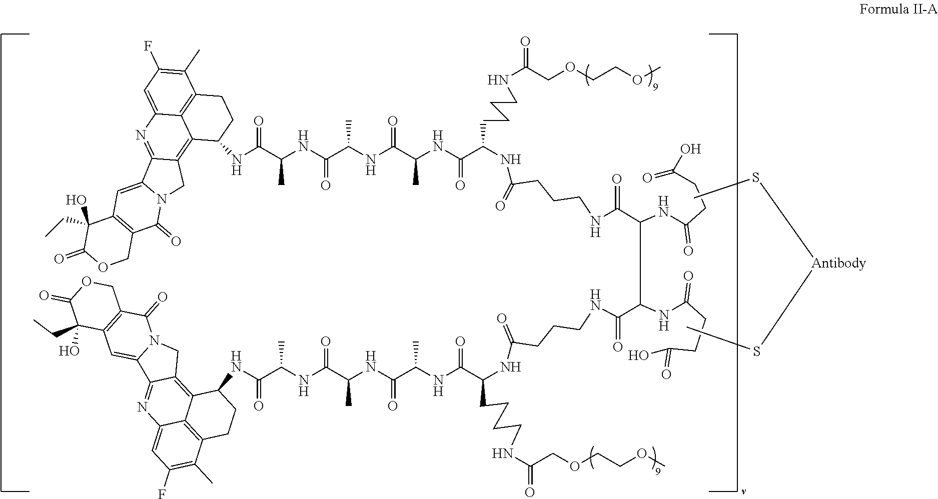
wherein the antibody is the anti-EGFR/c-Met bispecific antibody, v is 2; and
wherein conjugation is at the interchain cysteine residues making up the HC-LC disulfide bonds resulting in a DAR is 4.

20. A bispecific anti-EGFR/c-Met antibody drug conjugate (ADC) comprising a bispecific anti-EGFR/c-Met antibody that comprises:
a HC1 comprising a variable region (VH1) comprising the amino acid sequence of SEQ ID NO: 13;
a LC1 comprising a variable region (VL1) comprising the amino acid sequence of SEQ ID NO: 14;
a HC2 comprising a variable region (VH2) comprising the amino acid sequence of SEQ ID NO: 15; and
a LC2 comprising a variable region (VL2) comprising the amino acid sequence of SEQ ID NO: 16; and
wherein the bispecific anti-EGFR/c-Met antibody drug conjugate (ADC) comprises Formula II-A: 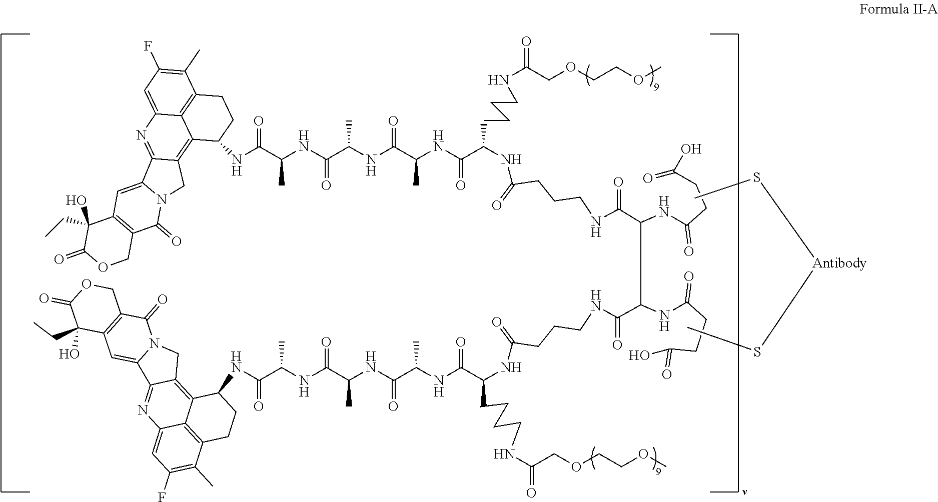
wherein the antibody is anti-EGFR/c-Met bispecific antibody, v is 2; and
wherein the bispecific anti-EGFR/c-Met antibody drug conjugate (ADC) comprises conjugation at the interchain cysteine residues making up the HC-LC disulfide bonds resulting in a DAR of 4.

21. A bispecific anti-EGFR/c-Met antibody drug conjugate (ADC) comprising a bispecific anti-EGFR/c-Met antibody that comprises:
a HC1 comprising the amino acid sequence of SEQ ID NO: 17;
a LC1 comprising the amino acid sequence of SEQ ID NO: 18;
a HC2 comprising the amino acid sequence of SEQ ID NO: 19; and
a LC2 comprising the amino acid sequence of SEQ ID NO: 20;
and wherein the bispecific anti-EGFR/c-Met antibody drug conjugate (ADC) comprises Formula II-A: 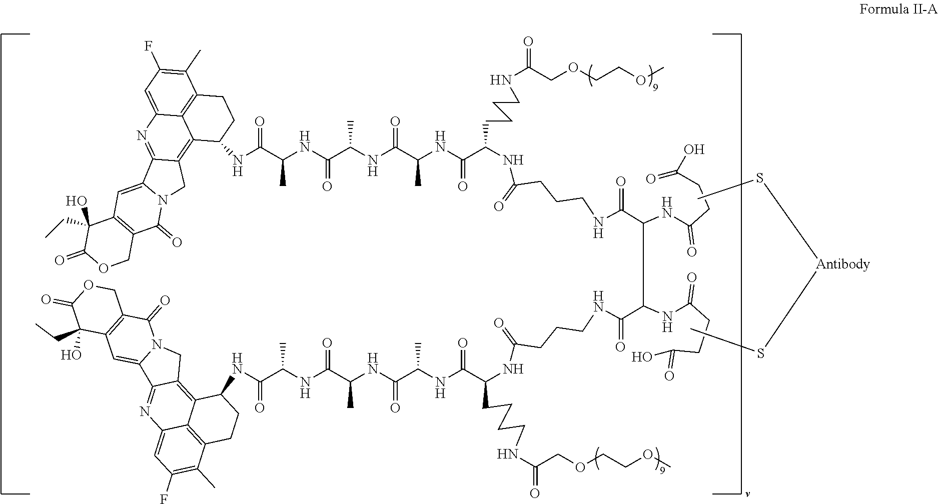
wherein the antibody is anti-EGFR/c-Met bispecific antibody, v is 2; and
wherein the bispecific anti-EGFR/c-Met antibody drug conjugate (ADC) comprises conjugation at the interchain cysteine residues making up the HC-LC disulfide bonds resulting in a DAR of 4.

22. A pharmaceutical composition comprising the bispecific anti-EGFR/c-Met ADC of any one of the previous claims and a pharmaceutically acceptable excipient.

23. A method of treating an EGFR expressing cancer and/or c-Met expressing cancer in a patient in need thereof, comprising administering the bispecific anti-EGFR/c-Met ADC of any one of claims 1-21 or the pharmaceutical composition of claim 22 to the patient to thereby treat the cancer.

24. The method of claim 23, wherein the cancer is lung cancer, colorectal cancer, or head and neck cancer.


SEQUENCE LISTING (68 sequences)

SEQ ID NO: 1 - protein, 5 aa, Homo sapiens
TYGMH

SEQ ID NO: 2 - protein, 17 aa, Homo sapiens
VIWDDGSYKYYGDSVKG

SEQ ID NO: 3 - protein, 16 aa, Homo sapiens
DGITMVRGVMKDYFDY

SEQ ID NO: 4 - protein, 11 aa, Homo sapiens
RASQDISSALV

SEQ ID NO: 5 - protein, 7 aa, Homo sapiens
DASSLES

SEQ ID NO: 6 - protein, 9 aa, Homo sapiens
QQFNSYPLT

SEQ ID NO: 7 - protein, 5 aa, Homo sapiens
SYGIS

SEQ ID NO: 8 - protein, 17 aa, Homo sapiens
WISAYNGYTNYAQKLQG

SEQ ID NO: 9 - protein, 10 aa, Homo sapiens
DLRGTNYFDY

SEQ ID NO: 10 - protein, 11 aa, Homo sapiens
RASQGISNWLA

SEQ ID NO: 11 - protein, 7 aa, Homo sapiens
AASSLLS

SEQ ID NO: 12 - protein, 9 aa, Homo sapiens
QQANSFPIT

SEQ ID NO: 13 - protein, 125 aa, Homo sapiens [sequence omitted]

SEQ ID NO: 14 - protein, 107 aa, Homo sapiens [sequence omitted]

SEQ ID NO: 15 - protein, 119 aa, Homo sapiens
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSS

SEQ ID NO: 16 - protein, 107 aa, Homo sapiens
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIK

SEQ ID NO: 17 - protein, 455 aa, Homo sapiens
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 18 - protein, 214 aa, Homo sapiens
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 449 aa, Homo sapiens
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 20 - protein, 214 aa, Homo sapiens
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 10 aa, synthetic construct
GFTFSTYGMH

SEQ ID NO: 22 - protein, 10 aa, synthetic construct
VIWDDGSYKY

SEQ ID NO: 23 - protein, 16 aa, synthetic construct
DGITMVRGVMKDYFDY

SEQ ID NO: 24 - protein, 11 aa, synthetic construct
RASQDISSALV

SEQ ID NO: 25 - protein, 7 aa, synthetic construct
DASSLES

SEQ ID NO: 26 - protein, 9 aa, synthetic construct
QQFNSYPLT

SEQ ID NO: 27 - protein, 7 aa, synthetic construct
GFTFSTY

SEQ ID NO: 28 - protein, 6 aa, synthetic construct
WDDGSY

SEQ ID NO: 29 - protein, 16 aa, synthetic construct
DGITMVRGVMKDYFDY

SEQ ID NO: 30 - protein, 11 aa, synthetic construct
RASQDISSALV

SEQ ID NO: 31 - protein, 7 aa, synthetic construct
DASSLES

SEQ ID NO: 32 - protein, 9 aa, synthetic construct
QQFNSYPLT

SEQ ID NO: 33 - protein, 8 aa, synthetic construct
GFTFSTYG

SEQ ID NO: 34 - protein, 8 aa, synthetic construct
IWDDGSYK

SEQ ID NO: 35 - protein, 18 aa, synthetic construct
ARDGITMVRGVMKDYFDY

SEQ ID NO: 36 - protein, 6 aa, synthetic construct
QDISSA

SEQ ID NO: 37 - skipped

SEQ ID NO: 38 - protein, 9 aa, synthetic construct
QQFNSYPLT

SEQ ID NO: 39 - protein, 6 aa, synthetic construct
STYGMH

SEQ ID NO: 40 - protein, 13 aa, synthetic construct
WVAVIWDDGSYKY

SEQ ID NO: 41 - protein, 17 aa, synthetic construct
ARDGITMVRGVMKDYFD

SEQ ID NO: 42 - protein, 7 aa, synthetic construct
SSALVWY

SEQ ID NO: 43 - protein, 10 aa, synthetic construct
LLIYDASSLE

SEQ ID NO: 44 - protein, 8 aa, synthetic construct
QQFNSYPL

SEQ ID NO: 45 - protein, 10 aa, synthetic construct
GYTFTSYGIS

SEQ ID NO: 46 - protein, 10 aa, synthetic construct
WISAYNGYTN

SEQ ID NO: 47 - protein, 10 aa, synthetic construct
DLRGTNYFDY

SEQ ID NO: 48 - protein, 11 aa, synthetic construct
RASQGISNWLA

SEQ ID NO: 49 - protein, 7 aa, synthetic construct
AASSLLS

SEQ ID NO: 50 - protein, 9 aa, synthetic construct
QQANSFPIT

SEQ ID NO: 51 - protein, 7 aa, synthetic construct
GYTFTSY

SEQ ID NO: 52 - protein, 6 aa, synthetic construct
SAYNGY

SEQ ID NO: 53 - protein, 10 aa, synthetic construct
DLRGTNYFDY

SEQ ID NO: 54 - protein, 11 aa, synthetic construct
RASQGISNWLA

SEQ ID NO: 55 - protein, 7 aa, synthetic construct
AASSLLS

SEQ ID NO: 56 - protein, 9 aa, synthetic construct
QQANSFPIT

SEQ ID NO: 57 - protein, 8 aa, synthetic construct
GYTFTSYG

SEQ ID NO: 58 - protein, 8 aa, synthetic construct
ISAYNGYT

SEQ ID NO: 59 - protein, 12 aa, synthetic construct
ARDLRGTNYFDY

SEQ ID NO: 60 - protein, 6 aa, synthetic construct
QGISNW

SEQ ID NO: 61 - skipped

SEQ ID NO: 62 - protein, 9 aa, synthetic construct
QQANSFPIT

SEQ ID NO: 63 - protein, 6 aa, synthetic construct
TSYGIS

SEQ ID NO: 64 - protein, 13 aa, synthetic construct
WMGWISAYNGYTN

SEQ ID NO: 65 - protein, 11 aa, synthetic construct
ARDLRGTNYFD

SEQ ID NO: 66 - protein, 7 aa, synthetic construct
SNWLAWF

SEQ ID NO: 67 - protein, 10 aa, synthetic construct
LLIYAASSLL

SEQ ID NO: 68 - protein, 8 aa, synthetic construct
QQANSFPI